(19) United States
(12) Patent Application Publication
(10) Pub. No.: US 2025/0223317 A1
(43) Pub. Date: Jul. 10, 2025

(54) KEAP1 INHIBITING PROTEIN-LIKE POLYMERS

(71) Applicant: NORTHWESTERN UNIVERSITY, Evanston, IL (US)

(72) Inventors: Nathan C. GIANNESCHI, Evanston, IL (US); Kendal Paige CARROW, Evanston, IL (US); Madeline Prindiville HOPPS, Evanston, IL (US)

(73) Assignee: NORTHWESTERN UNIVERSITY, Evanston, IL (US)

(21) Appl. No.: 18/850,729

(22) PCT Filed: Apr. 3, 2023
(86) PCT No.: PCT/US2023/065263

§ 371 Date: Sep. 25, 2024

Related U.S. Application Data
(60) Provisional application No. 63/327,071, filed on Apr. 4, 2022.

(51) Int. Cl. C07K 7/06; A61K 38/00

(52) U.S. Cl. CPC C07K 7/06; A61K 38/00

(57) ABSTRACT

In an aspect, the invention provides therapeutic agents comprising brush polymers that address challenges associated with conventional administration of free therapeutic peptides. In an embodiment, for example, the invention provides brush polymers incorporating one or more therapeutic peptides comprising a sequence having 75% or greater sequence identity with an 8 to 12 amino acid fragment of SEQ ID NO: 218 (MDLIDILWRQDIDLGVSREVFDFS). Therapeutic agents of the invention comprising brush polymers include high-density brush polymers including cross-linked brush polymers, brush block copolymers, and brush random copolymers. In an embodiment, brush polymers of the invention exhibit proteolysis-resistant characteristics and maintain their biological function during formulation and administration. The invention also includes methods of making and using therapeutic agents comprising brush polymers.

### CROSS-REFERENCE TO RELATED APPLICATIONS

This application claims the benefit of priority to U.S. Provisional Patent Application No. 63/327,071, filed Apr. 4, 2022, which is hereby incorporated by reference in its entirety.

### STATEMENT REGARDING FEDERALLY SPONSORED RESEARCH OR DEVELOPMENT

This invention was made with government support under Award Number 1F30AG076317-01A1, awarded by the National Institute of Health. The government has certain rights in the invention.

### REFERENCE TO AN ELECTRONIC SEQUENCE LISTING

The contents of the electronic sequence listing (339563_5-22_WO_KEAP1_Inhibition.xml; Size: 197,137 bytes; and Date of Creation: Mar. 20, 2023) is herein incorporated by reference in its entirety.

### BACKGROUND OF INVENTION

The use of protein and peptide therapeutics continues to increase dramatically for diverse clinical applications. However, inefficiencies in cellular uptake and rapid digestion by proteases are two problems that have limited the clinical adoption of peptide-based therapeutics. Accordingly, many peptide therapeutics are incompatible with systemic administration and, therefore, must be administered by injection at the site of action due to poor in vivo stability. This can result in poor patient compliance and, as such, many peptide therapies only are used clinically as salvage treatments.

One particular area of interest is in developing therapeutics targeting the protein-protein interaction between Nuclear factor (erythroid-derived 2)-like 2 (Nrf2) and Kelch-like ECH-Associating protein 1 (Keap1).

Nrf2 is a key regulator of cellular redox homeostasis and its dysfunction is implicated in many disease states. The decreased availability of Nrf2 in some disease states limits its protective role. Nrf2 maintains homeostasis by induction of the nuclear antioxidant response element (ARE), an enhancer located near many detoxifying and antioxidant genes. Nrf2 is regulated by homodimers of Keap1 which bind the low affinity DLG motif and high affinity ETGE motif of Nrf2 at each Kelch domain. See, for example, FIGS. 1A, 1, and 2A-2C and Fukutomi et al. (Mol. Cell. Biol., 34(5): 832-846 (2014)). Under physiologic conditions, Nrf2 is bound to Keap1 in the cytoplasm, which marks Nrf2 for degradation through ubiquitination. However, in the setting of cellular stress, Keap1 undergoes conformational changes leading to disruption of the binding, prevention of Nrf2 degradation, nuclear accumulation of Nrf2, and ARE activation. Given the role of aging, stress, and Nrf2 dysfunction in NDs, several inhibitors of the Keap1/Nrf2 protein-protein interaction (PPI) have been explored as novel therapeutics for diseases in which oxidative stress plays a role in pathophysiology. As such, the Keap1/Nrf2 interaction is important in a number of conditions including, for example, neurodegenerative diseases such as Alzheimer's and Parkinson's disease as well as heart and skin diseases among others. A therapeutic that successfully inhibits Keap1/Nrf2 binding can enhance the antioxidant and anti-inflammatory response to provide cytoprotective effects for a number of disease states.

Most existing small molecule inhibitors (SMIs), such as tert-butylhydroquinone (tBHQ), are indirect Keap1 inhibitors by acting as electrophiles and result in off-target effects. Direct Keap1 SMIs still have relatively low specificity for targeting large PPI interfaces; thus, peptide-based direct Keap1 inhibitors are of interest. Derivatives of the peptide sequence, LDEETGEFL, have been identified from the Keap1-binding region of Nrf2. Unfortunately, these peptide inhibitors face significant challenges for clinical translation. In this regard, Colarusso et al. (Bioorganic Med. Chem., 28; 1-12 (2020)) discloses the optimization of linear and cyclic peptide inhibitors of Keap1/Nrf2 protein-protein interaction. More particularly, Colarusso et al. generates a library of linear peptides based on the Nrf2-binding motif SEQ ID NO: 77 (LDEETGEFL). However, the linear and cyclic peptide inhibitors of Keap1/Nrf2, disclosed by Colarusso et al. suffer from substantial lack of cell permeability and were inactive. Furthermore, most peptide-based therapeutics only mimic the high affinity ETGE domain. Yet, native Nrf2 has two Keap1 binding regions, the high affinity ETGE binding domain and the low affinity DLG binding domain. Exploration into the DLG domain binding with the Kelch domain has identified the DLG motif SEQ ID NO: 225 (LWRQDIDLG) and the broader DIDLID element SEQ ID NO: 224 (MDLIDILWRQDIDLGV) as being foundational residues. The individual utility of these free peptide sequences alone has been limited and largely unstudied. Moreover, without modification utility of these free peptide sequences would be limited by cell impermeability. Thus, there remains a need for therapeutics targeting the DLG and/or ETGE domains of the protein-protein interaction between Nuclear factor (erythroid-derived 2)-like 2 (Nrf2) and Kelch-like ECH-Associating protein 1 (Keap1), which are cell permeable and enhance cellular Nrf2 activity.

Once the inefficiencies of cellular uptake are addressed, there remains the issue of digestion by proteases. Several approaches for producing peptides protected from proteolysis involve chemical modification of the amino acid sequence, which generally necessitates multiple rounds of structure-function studies to confirm that the activity of the peptide is not altered. Other approaches not using chemical modification of the amino acid sequence involve conjugation of the peptide to a pre-formed higher molecular weight structure, such as a polymer or nanomaterial. The downside of these approaches includes requiring additional conjugation and purification steps, as well as the formation of, and release from, the high molecular weight carrier.

Despite these challenges, there remaining significant interest in developing improved delivery systems to enhance clinical applicability and overall efficacy for therapies involving therapeutic peptides. Thus, there remains a need for delivery systems and methods for therapeutic peptides, such as those targeting the protein-protein interaction between Nrf2 and Keap1, which provide improved pharmacokinetic properties, administration routes and overall efficacies.

### SUMMARY OF THE INVENTION

In an aspect, the invention provides a peptide having a sequence having 75% or greater sequence (e.g., 80% or greater, 85% or greater, 90% or greater, 95% or greater, or 100%) identity with a 6 to 10 amino acid fragment of SEQ ID NO: 218 (MDLIDILWRQDIDLGVSREVFDFS), wherein the peptide further comprises a charge modulating domain having from 1 to 7 amino acid residues. In certain embodiments, the peptide comprises a sequence having 85% or greater (e.g., 90% or greater, 95% or greater, or 100%) sequence identity with a 6 to 10 amino acid fragment of SEQ ID NO: 218 (MDLIDILWRQDIDLGVSREVFDFS). In preferred embodiments, the peptide comprises a 6 to 10 amino acid fragment of SEQ ID NO: 218 (MDLIDILWRQDIDLGVSREVFDFS).

In an aspect, the provides a peptide having from 11 to 17 amino acid residues comprising a sequence having 85% or greater (e.g., 90% or greater, 95% or greater, or 100%) sequence identity of SEQ ID NO: 1 (RQDIDLGVSR), SEQ ID NO: 220 (ILWRQDIDLGVSR), or SEQ ID NO: 222 (LWRQDIDLGVSR), wherein the peptide further comprises a charge modulating domain having from 1 to 7 amino acid residues. In some embodiments, the sequence having 85% or greater (e.g., 90% or greater, 95% or greater, or 100%) sequence identity of SEQ ID NO: 1 (RQDIDLGVSR), SEQ ID NO: 220 (ILWRQDIDLGVSR), or SEQ ID NO: 222 (LWRQDIDLGVSR) is SEQ ID NO: 1 (RQDIDLGVSR), SEQ ID NO: 220 (ILWRQDIDLGVSR), or SEQ ID NO: 222 (LWRQDIDLGVSR). Typically, the charge modulating domain is a glycine-serine domain, a cationic residue domain, or a combination thereof. For example, the invention can provide a peptide having from 11 to 17 amino acid residues comprising a sequence having 85% or greater (e.g., 90% or greater, 95% or greater, or 100%) sequence identity of SEQ ID NO: 1 (RQDIDLGVSR), wherein the peptide further comprises a charge modulating domain having from 1 to 7 amino acid residues. In some embodiments, the sequence having 85% or greater (e.g., 90% or greater, 95% or greater, or 100%) sequence identity of SEQ ID NO: 1 (RQDIDLGVSR) is SEQ ID NO: 1 (RQDIDLGVSR). Typically, the charge modulating domain is a glycine-serine domain, a cationic residue domain, or a combination thereof.

In some aspects, the present invention further provides a peptide comprising a sequence having 75% or greater (e.g., 80% or greater, 85% or greater, 90% or greater, 95% or greater, or 100%) sequence identity of SEQ ID NO: 1-SEQ ID: 76. In certain aspects, the present invention provides a peptide selected from: SEQ ID NO: 2 (RQDIDLGVSRR), SEQ ID NO: 3 (RRQDIDLGVSR), SEQ ID NO: 4 (RQDIDLGVSRK), SEQ ID NO: 5 (KRQDIDLGVSR), SEQ ID NO: 6 (KRQDIDLGVSRR), SEQ ID NO: 7 (RRQDIDLGVSRK), SEQ ID NO: 8 (RQDIDLGVSRRR), SEQ ID NO: 9 (RQDIDLGVSRRRR), SEQ ID NO: 10 (RQDIDLGVSRRRRR), SEQ ID NO: 11 (RQDIDLGVSRRRRRR), SEQ ID NO: 12 (RRRQDIDLGVSR), SEQ ID NO: 13 (RRRRQDIDLGVSR), SEQ ID NO: 14 (RRRRRQDIDLGVSR), SEQ ID NO: 15 (RRRRRRQDIDLGVSR), SEQ ID NO: 16 (RRRQDIDLGVSRRR), SEQ ID NO: 17 (RRRRQDIDLGVSRRRR), SEQ ID NO: 18 (RRQDIDLGVSRR), SEQ ID NO: 19 (RRQDIDLGVSRRR), SEQ ID NO: 20 (RRRQDIDLGVSRR), SEQ ID NO: 21 (RRQDIDLGVSRRRR), SEQ ID NO: 22 (RRRRQDIDLGVSRR), SEQ ID NO: 23 (RRQDIDLGVSRRRRR), SEQ ID NO: 24 (RRRQDIDLGVSRRRR), SEQ ID NO: 25 (RRRRQDIDLGVSRRR), SEQ ID NO: 26 (RRRRRQDIDLGVSRR), SEQ ID NO: 27 (RRQDIDLGVSRRRRRR), SEQ ID NO: 28 (RRRQDIDLGVSRRRRR), SEQ ID NO: 29 (RRRRRQDIDLGVSRRR), SEQ ID NO: 30 (RRRRRRQDIDLGVSRR), SEQ ID NO: 31 (RQDIDLGVSRKK), SEQ ID NO: 32 (RQDIDLGVSRKKK), SEQ ID NO: 33 (RQDIDLGVSRKKKK), SEQ ID NO: 34 (RQDIDLGVSRKKKKK), SEQ ID NO: 35 (KKRQDIDLGVSR), SEQ ID NO: 36 (KKKRQDIDLGVSR), SEQ ID NO: 37 (KKKKRQDIDLGVSR), SEQ ID NO: 38 (KKKKKRQDIDLGVSR), SEQ ID NO: 39 (KKRQDIDLGVSRKK), SEQ ID NO: 40 (KKKRQDIDLGVSRKKK), SEQ ID NO: 41 (KRQDIDLGVSRK), SEQ ID NO: 42 (KRQDIDLGVSRKK), SEQ ID NO: 43 (KKRQDIDLGVSRK), SEQ ID NO: 44 (KRQDIDLGVSRKKK), SEQ ID NO: 45 (KKKRQDIDLGVSRK), SEQ ID NO: 46 (KRQDIDLGVSRKKKK), SEQ ID NO: 47 (KKRQDIDLGVSRKKK), SEQ ID NO: 48 (KKKRQDIDLGVSRKK), SEQ ID NO: 49 (KKKKRQDIDLGVSRK), SEQ ID NO: 50 (KRQDIDLGVSRKKKKK), SEQ ID NO: 51 (KKRQDIDLGVSRKKKK), SEQ ID NO: 52 (KKKKRQDIDLGVSRKK), SEQ ID NO: 53 (KKKKKRQDIDLGVSRK), SEQ ID NO: 54 (RQDIDLGVSRKRKR), SEQ ID NO: 55 (KRKRRQDIDLGVSR), SEQ ID NO: 56 (RKRKRQDIDLGVSR), SEQ ID NO: 57 (RQDIDLGVSRRKRK), SEQ ID NO: 58 (KKRQDIDLGVSRRR), SEQ ID NO: 59 (RRRQDIDLGVSRKK), SEQ ID NO: 60 (KRQDIDLGVSRRRR), SEQ ID NO: 61 (KKKRQDIDLGVSRR), SEQ ID NO: 62 (RRRRQDIDLGVSRK), SEQ ID NO: 63 (KRRQDIDLGVSRKR), SEQ ID NO: 64 (RKRQDIDLGVSRRK), SEQ ID NO: 65 (RKRQDIDLGVSRKR), SEQ ID NO: 66 (KRRQDIDLGVSRRK), SEQ ID NO: 67 (RQDIDLGVSRKKRR), SEQ ID NO: 68 (RQDIDLGVSRRRKK), SEQ ID NO: 69 (KKRRRQDIDLGVSR), SEQ ID NO: 70 (RRKKRQDIDLGVSR), SEQ ID NO: 71 (RQDIDLGVSRGSGSGRR), SEQ ID NO: 72 (GSGSGRRRQDIDLGVSR), SEQ ID NO: 73 (RQDIDLGVSRGSGSGKK), SEQ ID NO: 74 (GSGSGKKRQDIDLGVSR), SEQ ID NO: 75 (YGRKKRRRQDIDLGVSR), and SEQ ID NO: 76 (RQDIDLGVSRYGRKKRR).

The present invention further includes brush polymer therapeutic agents comprising a peptide comprising a sequence having 75% or greater sequence (e.g., 80% or greater, 85% or greater, 90% or greater, 95% or greater, or 100%) identity with a 6 to 10 amino acid fragment of SEQ ID NO: 218 (MDLIDILWRQDIDLGVSREVFDFS), including drugs and prodrugs thereof, which address challenges associated with conventional administration of such a therapeutic peptide. In some embodiments, the present invention includes brush polymer therapeutic agents comprising a peptide having a sequence having 75% or greater (e.g., 80% or greater, 85% or greater, 90% or greater, 95% or greater, or 100%) sequence identity of SEQ ID NO: 1 (RQDIDLGVSR), SEQ ID NO: 220 (ILWRQDIDLGVSR), or SEQ ID NO: 222 (LWRQDIDLGVSR). For example, the present invention can include brush polymer therapeutic agents comprising a peptide comprising a sequence having 75% or greater (e.g., 80% or greater, 85% or greater, 90% or greater, 95% or greater, or 100%) sequence identity of SEQ ID NO: 1 (RQDIDLGVSR), including drugs and prodrugs thereof, which address challenges associated with conventional administration of such a therapeutic peptide.

In an embodiment, for example, the invention provides brush polymers (e.g., therapeutic polymers or therapeutic agents) incorporating one or more peptides comprising a sequence having 75% or greater (e.g., 80% or greater, 85% or greater, 90% or greater, 95% or greater, or 100%) sequence identity of SEQ ID NO: 1 (RQDIDLGVSR) as side chain moieties. Brush polymers of some embodiments are characterized by high brush densities, including optionally cross-linked brush polymers, brush block copolymers, or brush random copolymers. Brush polymers of the invention include brush polymers having polymer side chains characterized by one or more degradable linker, such as an in vivo degradable linker or triggerable linker.

In an embodiment, brush polymers of the invention exhibit proteolysis-resistant characteristics and maintain their biological function during formulation and in vivo administration to a subject. In some embodiments, conjugation of the therapeutic peptide to the brush polymer backbone renders it more resistant to in vivo degradation by proteolytic enzymes as compared to a free therapeutic peptide. Moreover, the higher molecular weight of the brush polymer, relative to its free therapeutic peptide analogue, confers longer circulation time than the free therapeutic peptide. As a result, the therapeutic polymers can be administered less frequently and in smaller doses than the free peptide therapeutics used in the clinic. Further, the enhanced stability and resistance to degradation of the present brush polymer therapeutic agents allows for more versatility with respect to administration route and conditions, including in injection at the site of action and systemic administration. Alternatively, or in addition to, the brush polymers of the invention may exhibit stronger binding affinity than the free peptide

The invention also includes methods of using brush polymers for a range of clinical applications including, by way of example, for treatment or management of conditions associated with an inflammatory state, increased oxidative stress, autoimmune pathophysiology, chemo-preventative measures, neurodegeneration, or a combination thereof. More particularly, the brush polymers described herein can be used for treatment or management of autoimmune disease, respiratory disease, gastrointestinal disease, cardiovascular disease, or neurodegenerative disease.

The invention also includes methods for making therapeutic agents comprising brush polymers, for example, via “grafting from” methods, “grafting onto” methods and “grafting through” methods. In some methods, a ring opening metathesis polymerization (ROMP) synthetic approach is used to make therapeutic agents comprising brush polymers, for example, having high graft densities and low polydispersity. The present methods of making therapeutic agents comprising brush polymers include other non-ROMP synthetic pathways such as, by way of example, reversible addition fragmentation chain transfer (RAFT) polymerization, stable free radical mediated polymerization and atom transfer radical polymerization (ATRP).

In an embodiment, the invention provides a polymer comprising a polymer comprising a first polymer segment at least 2 first repeating units and optionally 2-30, 5-30, 10-30, 15-30, or 20-30 first repeating units; wherein each of the first repeating units of the first polymer segment comprises a first polymer backbone group directly or indirectly covalently linked to a first polymer side chain group comprising a peptide; wherein the peptide comprises a sequence having 75% or greater (e.g., 80% or greater, 85% or greater, 90% or greater, 95% or greater, or 100%) sequence identity of SEQ ID NO: 1 (RQDIDLGVSR), SEQ ID NO: 220 (ILWRQDIDLGVSR), or SEQ ID NO: 222 (LWRQDIDLGVSR), optionally wherein the peptide comprises SEQ ID NO: 1 (RQDIDLGVSR), SEQ ID NO: 220 (TLWRQDIDLGVSR), or SEQ ID NO: 222 (LWRQDIDLGVSR). In certain embodiments, the peptide further comprises a charge modulating domain having from 1 to 7 amino acid residues and/or has a total of from 11 to 17 amino acid residues.

In an aspect, a polymer is provided, the polymer is characterized by the formula (FX2a), (FX2b), or (FX2c):

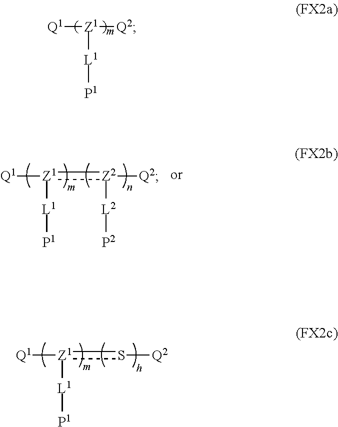

wherein each Z¹ is independently a first polymer backbone group and each Z² is independently a second polymer backbone group; each S is independently a repeating unit having a composition different from the first repeating unit; Q¹ is a first backbone terminating group and Q² is a second backbone terminating group; each L¹ is independently a first linking group, each L² is independently a second linking group; each P¹ is the peptide; wherein each P² is a polymer side chain having a composition different from that of P¹; each m is independently an integer selected from the range of 2 to 1000; each n is independently an integer selected from the range of 0 to 1000; and each h is independently an integer selected from the range of 0 to 1000, wherein P¹ comprises a sequence having 75% or greater (e.g., 80% or greater, 85% or greater, 90% or greater, 95% or greater, or 100%) sequence identity of SEQ ID NO: 1 (RQDIDLGVSR), SEQ ID NO: 220 (ILWRQDIDLGVSR), or SEQ ID NO: 222 (LWRQDIDLGVSR), optionally wherein P¹ comprises SEQ ID NO: 1 (RQDIDLGVSR), SEQ ID NO: 220 (ILWRQDIDLGVSR), or SEQ ID NO: 222 (LWRQDIDLGVSR). In certain embodiments, P¹ further comprises a charge modulating domain having from 1 to 7 amino acid residues and/or P¹ has a total of from 11 to 17 amino acid residues.

In an aspect, a polymer is provided comprising: a first polymer segment comprising at least 2 first repeating units; wherein each of the first repeating units of the first polymer segment comprises a first polymer backbone group directly or indirectly covalently linked to a first polymer side chain group comprising a peptide; wherein the peptide interrupts the protein-protein interaction between Nuclear factor (erythroid-derived 2)-like 2 (Nrf2) and Kelch-like ECH-Associating protein 1 (Keap1) (e.g., by inhibiting binding to the DLG motif of Nrf2); and wherein the polymer exhibits efficacy for treatment or management of a condition associated with an inflammatory state, increased oxidative stress, autoimmune pathophysiology, chemo-preventative measures, neurodegeneration, or a combination thereof. In an embodiment of this aspect, the peptide comprises a sequence having 75% or greater sequence identity, optionally 80% or greater, 85% or greater, 90% or greater, 95% or greater, or 100%, of SEQ ID NO: 1 (RQDIDLGVSR), SEQ ID NO: 220 (ILWRQDIDLGVSR), or SEQ ID NO: 222 (LWRQDIDLGVSR). In an embodiment of this aspect, the peptide comprises SEQ ID NO: 1 (RQDIDLGVSR), SEQ ID NO: 220 (ILWRQDIDLGVSR), or SEQ ID NO: 222 (LWRQDIDLGVSR). In certain embodiments, the peptide comprises a sequence having 85% or greater sequence identity of SEQ ID NO: 1 (RQDIDLGVSR). In preferred embodiments, the peptide comprises SEQ ID NO: 1 (RQDIDLGVSR).

In an aspect, a polymer is provided comprising: a first polymer segment comprising at least 2 first repeating units; wherein each of the first repeating units of the first polymer segment comprises a first polymer backbone group directly or indirectly covalently linked to a first polymer side chain group comprising a peptide; wherein the peptide is selected from SEQ ID NO: 1-SEQ ID NO: 76. In an embodiment of this aspect, the peptide interrupts the protein-protein interaction between Nuclear factor (erythroid-derived 2)-like 2 (Nrf2) and Kelch-like ECH-Associating protein 1 (Keap1) (e.g., by inhibiting binding to the DLG motif of Nrf2), optionally for the treatment or management of a condition associated with an inflammatory state, increased oxidative stress, autoimmune pathophysiology, chemo-preventative measures, neurodegeneration, or a combination thereof.

In an embodiment, the polymer is a homopolymer or a copolymer. In an embodiment, the polymer is a brush polymer, optionally a brush block copolymer or a brush random copolymer. In an embodiment, the first polymer segment of the polymer comprises at least 5 first repeating units, optionally 5-30 first repeating units. In an embodiment, the polymer is characterized by a degree of polymerization of 2 to 1000 (e.g., a degree of polymerization of 2 to 100, a degree of polymerization of 5 to 100, a degree of polymerization of 2 to 50, a degree of polymerization of 5 to 50, a degree of polymerization of 2 to 25, or a degree of polymerization of 5 to 25). In an embodiment, the polymer is characterized by a polydispersity index less than 1.75 (e.g., a polydispersity index less than 1.5 or a polydispersity index less than 1.25).

In an embodiment, the peptide has from 11 to 17 amino acid residues, optionally from 11 to 16 amino acid residues, from 11 to 15 amino acid residues, or from 11 to 14 amino acid residues. In an embodiment, the peptide is a branched peptide, a linear peptide, cyclic peptide, or a cross-linked peptide. In an embodiment, the polymer is characterized by a structure wherein at least a portion of the peptide is linked to the polymer backbone group via an enzymatically degradable linker, such a matrix metalloproteinase (MMP) cleavage sequence, cathepsin B cleavage sequence, ester bond, reductive sensitive bond-disulfide bond, pH sensitive bond-imine bond or any combinations of these. In an embodiment, the polymer is characterized by a structure wherein at least a portion of the peptide side-chain is linked to the polymer backbone or consists of a degradable or triggerable linker. In some embodiments, the peptide and/or polymer further comprises a tag for imaging and/or analysis. For example, the peptide and/or polymer can further comprise a dye, radiolabeling, an imaging agent, tritiation, and the like.

In an embodiment, the polymer is characterized by the formula (FX1a), (FX1b), (FX1c), (FX1d); (FX1e); (FX1f); or (FX1g):

Q¹-T-Q² (FX1a);
Q¹-T-[S]ₕ-Q² (FX1b);
Q¹-[S]ₕ-T-Q² (FX1c);
Q¹-[S]ᵢ-T-[S]ₕ-Q² (FX1d);
Q¹-[S]ᵢ-T-[S]ₕ-T-Q² (FX1e);
Q¹-T-[S]ᵢ-T-[S]ₕ-Q² (FX1f);
or
Q¹-T-[S]ᵢ-T-[S]ₕ-T-Q² (FX1g);

wherein each T is independently the first polymer segment comprising the first repeating units and each S is independently an additional polymer segment; Q¹ is a first backbone terminating group; Q² is a second backbone terminating group; and wherein h is zero or an integer selected over the range of 1 to 1000 and i is zero or an integer selected over the range of 1 to 1000. In an embodiment, the polymer is characterized by any of formulas (FX1a)-(FX1g), wherein each -T- is independently —[Y¹]ₘ—; wherein each Y¹ is independently the first repeating unit of the first polymer segment; and each m is independently an integer selected from the range 0 to 1000, provided that at least one m is an integer selected from the range 1 to 1000.

In an embodiment, the polymer is characterized by the formula (FX2a), (FX2b), or (FX2c):

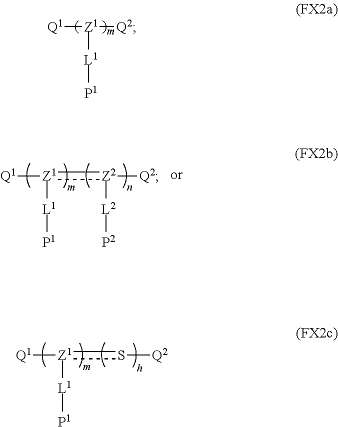

wherein each Z¹ is independently a first polymer backbone group and each Z² is independently a second polymer backbone group; wherein each S is independently a repeating unit having a composition different from the first repeating unit; the wherein Q¹ is a first backbone terminating group and Q² is a second backbone terminating group; wherein each L¹ is independently a first linking group, each L² is independently a second linking group; wherein each P¹ is the polymer side chain comprising the peptide; wherein each P² is a polymer side chain having a composition different from that of P¹; and wherein each m is independently an integer selected from the range of 2 to 1000 (e.g., 2 to 500, 2 to 250, or 2 to 100); wherein each n is each independently an integer selected from the range of 0 to 1000 (e.g., 0 to 500, 0 to 250, or 0 to 100); and wherein h are each independently an integer selected from the range of 0 to 1000 (e.g., 0 to 500, 0 to 250, or 0 to 100). In certain embodiments, each of the first polymer backbone group and/or the second polymer backbone group is a substituted or unsubstituted polymerized norbornene, olefin, cyclic olefin, norbornene anhydride, cyclooctene, cyclopentadiene, styrene, acrylamide, or acrylate.

In an embodiment, the polymer is characterized by any of formulas (FX2a)-(FX2c), wherein each Z¹ connected to L¹, and P¹ or a combination thereof is independently characterized by the formula (FX3a) or (FX3b):

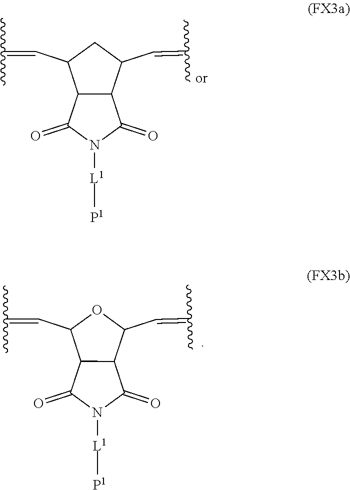

and wherein each Z² connected to L², and P² or a combination thereof is independently characterized by the formula (FX4a) or (FX4b)

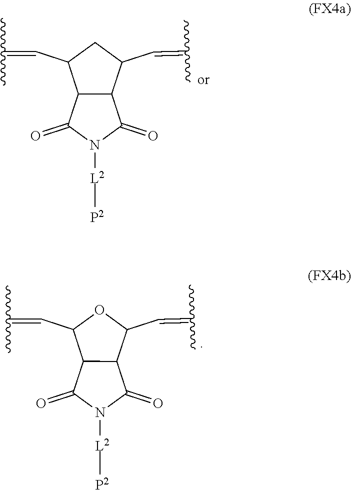

In an embodiment, the polymer is characterized by any of formulas (FX2a)-(FX2c), wherein each of Z¹ and Z² is independently a substituted or unsubstituted norbornene, oxanorbornene, olefin, cyclic olefin, cyclooctene, or cyclopentadiene. In an embodiment, the polymer is characterized by any of formulas (FX2a)-(FX2c), wherein each of Q¹ and Q² is independently selected from a hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀alkylaryl, —CO₂R³, —CONR⁴R⁵, —COR⁶, —SOR⁷, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ is independently H, C₅-C₁₀ aryl or C₁-C₁₀ alkyl. In an embodiment, the polymer is characterized by any of formulas (FX2a)-(FX2c), wherein each of L¹ and L² is independently selected from a single bond, an oxygen, and groups having an alkyl group, an alkenylene group, an arylene group, an alkoxy group, an acyl group, a triazole group, a diazole group, a pyrazole group, and combinations thereof. In an embodiment, the polymer is characterized by any of formulas (FX2a)-(FX2c), wherein each of L¹ and L² is independently selected from a single bond, —O—, —C₁-C₁₀ alkyl, C₂-C₁₀ alkenylene, C₃-C₁₀ arylene, C₁-C₁₀ alkoxy, C₁-C₁₀ acyl and combinations thereof.

In an embodiment, the polymer is characterized by any of formulas (FX2a)-(FX2c), wherein P¹ comprises a sequence having 75% or greater sequence identity, optionally 80% or greater, 85% or greater, 90% or greater, 95% or greater, or 100%, of SEQ ID NO: 1 (RQDIDLGVSR), SEQ ID NO: 220 (ILWRQDIDLGVSR), or SEQ ID NO: 222 (LWRQDIDLGVSR). In certain embodiments, the polymer is characterized by any of formulas (FX2a)-(FX2c), wherein P¹ comprises SEQ ID NO: 1 (RQDIDLGVSR), SEQ ID NO: 220 (ILWRQDIDLGVSR), or SEQ ID NO: 222 (LWRQDIDLGVSR). In certain embodiments, P¹ comprises a sequence having 85% or greater sequence identity of SEQ ID NO: 1 (RQDIDLGVSR). In preferred embodiments, P¹ comprises SEQ ID NO: 1 (RQDIDLGVSR).

In an embodiment, the polymer is characterized by any of formulas (FX2a)-(FX2c), wherein P¹ is selected from SEQ ID NO: 1-SEQ ID NO: 76, optionally wherein P¹ is selected from SEQ ID NO: 2-SEQ ID NO: 76.

In an aspect, provided are methods of treatment comprising administering to a subject an effective amount of any of the polymers disclosed herein.

In an aspect, provided are methods of treating or managing a condition in a subject comprising: administering to a subject an effective amount of a polymer comprising: a first polymer segment comprising at least 2 first repeating units; wherein each of the first repeating units of the first polymer segment comprises a first polymer backbone group directly or indirectly covalently linked to a first polymer side chain group comprising a peptide; wherein the peptide interrupts the protein-protein interaction between Nuclear factor (erythroid-derived 2)-like 2 (Nrf2) and Kelch-like ECH-Associating protein 1 (Keap1) (e.g., by inhibiting binding to the DLG motif of Nrf2); wherein the condition is associated with an inflammatory state, increased oxidative stress, autoimmune pathophysiology, chemo-preventative measures, neurodegeneration, or a combination thereof.

In an aspect, provided are methods of treating or managing a condition in a subject comprising: administering to a subject an effective amount of a polymer comprising: a first polymer segment comprising at least 2 first repeating units; wherein each of the first repeating units of the first polymer segment comprises a first polymer backbone group directly or indirectly covalently linked to a first polymer side chain group comprising a peptide; wherein the peptide interrupts the protein-protein interaction between Nuclear factor (erythroid-derived 2)-like 2 (Nrf2) and Kelch-like ECH-Associating protein 1 (Keap1) (e.g., by inhibiting binding to the DLG motif of Nrf2); wherein the condition is an autoimmune disease (e.g., multiple sclerosis, systemic lupus erythematous, Sjogren syndrome, rheumatoid arthritis, vitiligo, or psoriasis), a respiratory disease (e.g., COPD, emphysema, potential treatment for smokers, idiopathic pulmonary fibrosis, chronic sarcoidosis, or hypersensitivity pneumonitis), a gastrointestinal disease (e.g., ulcerative colitis, ulcers, prevent acetaminophen toxicity, non-alcoholic steatohepatitis, primary biliary cholangitis, cirrhosis, type 2 diabetes, or diabetic nephropathy), a cardiovascular disease (e.g., cardiac ischemia-reperfusion injury, heart failure, or atherosclerosis), or a neurodegenerative disease (e.g., Alzheimer's disease, Parkinson's disease, amyotrophic lateral sclerosis (ALS), Huntington's disease, Friedreich ataxia, or frontotemporal lobar degeneration).

In an embodiment, any of the present methods further comprise contacting a target tissue of the subject with the polymer or a metabolite or product thereof. In an embodiment, any of the present methods further comprise contacting a target cell of the subject with the polymer or a metabolite or product thereof. In an embodiment, any of the present methods further comprise contacting a target receptor of the subject with the polymer or a metabolite or product thereof. In preferred embodiments of the methods described herein, the polymer passes through the cell membrane and contacts an intracellular target.

In some embodiments a polymer, a method, or a formulation disclosed herein, each of the first polymer backbone group and/or the second polymer backbone group is a polymerized acrylamide (e.g., acrylamide or methacrylamide) monomer. In certain embodiments of a polymer, a method, or a formulation disclosed herein, each of the first polymer backbone group and the second polymer backbone group is a polymerized acrylamide (e.g., acrylamide or methacrylamide) monomer. For example, for any of the polymers, methods, or formulations disclosed herein, each of the first polymer backbone group is a polymerized acrylamide (e.g., acrylamide or methacrylamide) monomer. Alternatively, or additionally, for any of the polymers, methods, or formulations disclosed herein, each of the second polymer backbone group of the polymer is a polymerized acrylamide (e.g., acrylamide or methacrylamide) monomer. Optionally in any embodiment of a polymer, a method, or a formulation disclosed herein, each of the first polymer backbone group and/or the second polymer backbone group comprises a polymerized acrylamide (e.g., acrylamide or methacrylamide) monomer. Optionally in any embodiment of a polymer, a method, or a formulation disclosed herein, each of the first polymer backbone group and the second polymer backbone group comprises a polymerized acrylamide (e.g., acrylamide or methacrylamide) monomer. Optionally in any embodiment of a polymer, a method, or a formulation disclosed herein, each of the first polymer backbone group comprises a polymerized acrylamide (e.g., acrylamide or methacrylamide) monomer. Optionally in any embodiment of a polymer, a method, or a formulation disclosed herein, each polymer backbone group of the polymer comprises a polymerized acrylamide (e.g., acrylamide or methacrylamide) monomer.

Preferably in any embodiment of a polymer, a method, or a formulation disclosed herein, each of the first polymer backbone group and/or the second polymer backbone group is a polymerized norbornene dicarboxyimide monomer. Preferably in any embodiment of a polymer, a method, or a formulation disclosed herein, each of the first polymer backbone group and the second polymer backbone group is a polymerized norbornene dicarboxyimide monomer. Preferably in any embodiment of a polymer, a method, or a formulation disclosed herein, each of the first polymer backbone group is a polymerized norbornene dicarboxyimide monomer. Preferably in any embodiment of a polymer, a method, or a formulation disclosed herein, each polymer backbone group of the polymer is a polymerized norbornene dicarboxyimide monomer. Optionally in any embodiment of a polymer, a method, or a formulation disclosed herein, each of the first polymer backbone group and/or the second polymer backbone group comprises a polymerized norbornene dicarboxyimide monomer. Optionally in any embodiment of a polymer, a method, or a formulation disclosed herein, each of the first polymer backbone group and the second polymer backbone group comprises a polymerized norbornene dicarboxyimide monomer. Optionally in any embodiment of a polymer, a method, or a formulation disclosed herein, each of the first polymer backbone group comprises a polymerized norbornene dicarboxyimide monomer. Optionally in any embodiment of a polymer, a method, or a formulation disclosed herein, each polymer backbone group of the polymer comprises a polymerized norbornene dicarboxyimide monomer.

Preferably in any embodiment of a polymer, a method, or a formulation disclosed herein, the polymer is stable against enzymatic digestion. Optionally in any embodiment of a polymer, a method, or a formulation disclosed herein, the polymer is stable against enzymatic digestion by a metalloproteinase. Optionally in any embodiment of a polymer, a method, or a formulation disclosed herein, the polymer is stable against enzymatic digestion by matrix metalloproteinases and thermolysin. Preferably in any embodiment of a polymer, a method, or a formulation disclosed herein, the polymer is stable against enzymatic digestion for at least 450 minutes. Optionally in any embodiment of a polymer, a method, or a formulation disclosed herein, the polymer is stable against enzymatic digestion by thermolysin such that less than 20% of thermolysin-cleavable sites are cleaved by thermolysin after at least 450 minutes of the polymer's exposure to thermolysin. Optionally in any embodiment of a polymer, a method, or a formulation disclosed herein, each polymer individually solvated by water when a plurality of said polymers is dispersed in water.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A depicts the Keap1/Nrf2 protein-protein interaction where the Kelch domains of the Keap1 homodimer interact with the DLG and ETGE domains of Nrf2.

FIG. 1B shows the Keap1/Nrf2 pathway under basal conditions, where Keap1 is degraded via ubiquination by Keap1, and under oxidative stress, where Nrf2 activates the ARE pathway.

FIGS. 2A-2C show the documented structural characteristics of the Keap1 interaction with the Nrf2 protein. FIG. 2A shows the intermolecular hydrogen bonds between Keap1-DC and the section of the Nrf2 protein with SEQ ID NO: 223 (LWRQDIDLGVSREV). FIG. 2B shows the intermolecular hydrogen bonds between Keap1-DC and the section of the Nrf2 protein with SEQ ID NO: 77 (LDEETGEFL). FIG. 2C shows the amino acid residues involved in the intermolecular interactions with Nrf2-DLG, Nrf2-ETGE, and both are green, magenta, and cyan, respectively. Cysteine residues are yellow. See Fukutomi et al. (Mol. Cell. Biol., 34(5): 832-846 (2014)).

FIG. 3 provides an in-silico model of the binding interaction between the Kelch domain of the Keap1 protein and a peptide with SEQ ID NO: 218 (MDLIDILWRQDIDLGVSREVFDFS), prepared using simulations with CABS-Dock software, which is capable of simultaneous prediction of binding sites and protein-peptide docking through coarse-gain methods, as described in Example 1.

FIG. 4 provides a contact map of the amino acid interactions of the interface between the Kelch domain of the Keap1 protein and the peptide with SEQ ID NO: 218 (MDLIDILWRQDIDLGVSREVFDFS), as depicted in FIG. 3 and described in Example 1.

FIG. 5 provides an in-silico model of the binding interaction between the Kelch domain of the Keap1 protein and a peptide with SEQ ID NO: 2 (RQDIDLGVSRR), prepared using simulations with CABS-Dock software, which is capable of simultaneous prediction of binding sites and protein-peptide docking through coarse-gain methods, as described in Example 2.

FIG. 6 provides a contact map of the amino acid interactions of the interface between the Kelch domain of the Keap1 protein and the peptide with SEQ ID NO: 2 (RQDIDLGVSRR), as depicted in FIG. 5 and described in Example 2.

FIG. 7 provides an in-silico model of the binding interaction between the Kelch domain of the Keap1 protein and a peptide with SEQ ID NO: 1 (RQDIDLGVSR), prepared using simulations with CABS-Dock software, which is capable of simultaneous prediction of binding sites and protein-peptide docking through coarse-gain methods, as described in Example 3.

FIG. 8 provides a contact map of the amino acid interactions of the interface between the Kelch domain of the Keap1 protein and the peptide with SEQ ID NO: 1 (RQDIDLGVSR), as depicted in FIG. 7 and described in Example 3.

FIG. 9 provides an in-silico model of the binding interaction between the Kelch domain of the Keap1 protein and a peptide with SEQ ID NO: 219 (TLWRQDIDLGVSRR), prepared using simulations with CABS-Dock software, which is capable of simultaneous prediction of binding sites and protein-peptide docking through coarse-gain methods, as described in Example 4.

FIG. 10 provides a contact map of the amino acid interactions of the interface between the Kelch domain of the Keap1 protein and the peptide with SEQ ID NO: 219 (ILWRQDIDLGVSRR), as depicted in FIG. 9 and described in Example 4.

FIG. 11 provides an in-silico model of the binding interaction between the Kelch domain of the Keap1 protein and a peptide with SEQ ID NO: 220 (ILWRQDIDLGVSR), prepared using simulations with CABS-Dock software, which is capable of simultaneous prediction of binding sites and protein-peptide docking through coarse-gain methods, as described in Example 5.

FIG. 12 provides a contact map of the amino acid interactions of the interface between the Kelch domain of the Keap1 protein and the peptide with SEQ ID NO: 220 (ILWRQDIDLGVSR), as depicted in FIG. 11 and described in Example 5.

FIG. 13 provides an in-silico model of the binding interaction between the Kelch domain of the Keap1 protein and a peptide with SEQ ID NO: 221 (LWRQDIDLGVSRR), prepared using simulations with CABS-Dock software, which is capable of simultaneous prediction of binding sites and protein-peptide docking through coarse-gain methods, as described in Example 6.

FIG. 14 provides a contact map of the amino acid interactions of the interface between the Kelch domain of the Keap1 protein and the peptide with SEQ ID NO: 221 (LWRQDIDLGVSRR), as depicted in FIG. 13 and described in Example 6.

FIG. 15 provides an in-silico model of the binding interaction between the Kelch domain of the Keap1 protein and a peptide with SEQ ID NO: 222 (LWRQDIDLGVSR), prepared using simulations with CABS-Dock software, which is capable of simultaneous prediction of binding sites and protein-peptide docking through coarse-gain methods, as described in Example 7.

FIG. 16 provides a contact map of the amino acid interactions of the interface between the Kelch domain of the Keap1 protein and the peptide with SEQ ID NO: 222 (LWRQDIDLGVSR), as depicted in FIG. 15 and described in Example 7.

FIG. 17 shows the predicted Kelch-peptide complex structures using Alphafold-multimer for peptides SEQ ID NO: 220 (ILWRQDIDLGVSR), SEQ ID NO: 219 (ILWRQDIDLGVSRR), SEQ ID NO: 222 (LWRQDIDLGVSR), SEQ ID NO: 221 (LWRQDIDLGVSRR), SEQ ID NO: 1 (RQDIDLGVSR), SEQ ID NO: 2 (RQDIDLGVSRR), and SEQ ID NO: 218 (MDLIDILWRQDIDLGVSREVFDFS). Shown in the top row of FIG. 17 are the five predicted structures (Ranks 1-5) and the experimental structure of SEQ ID NO: 218 (MDLIDILWRQDIDLGVSREVFDFS), which are aligned using the backbone atoms of the Kelch domain only. Also included are the model confidence scores (1≥DockQ≥0) for the five predicted structures where a higher score (e.g., Rank 1) stands for a higher confidence. Shown in the bottom row of FIG. 17 are the five predicted structures (ribbon depiction) for each peptide, superimposed with the experimental structure of SEQ ID NO: 218 (MDLIDILWRQDIDLGVSREVFDFS).

FIG. 18A shows the ring-opening metathesis polymerization (ROMP) of a polynorbornene dicarboxyimide monomer comprising SEQ ID NO: 2 (RQDIDLGVSRR) to form a polynorbornene dicarboxyimide-based brush polymer comprising the peptide SEQ ID NO: 2 (RQDIDLGVSRR), as described in Example 10.

FIG. 18B shows the mass spectrum for the polynorbornene dicarboxyimide monomer comprising SEQ ID NO: 2 (RQDIDLGVSRR) as measured by electrospray ionization (ESI) mass spectrometry.

FIG. 18C shows the high-performance liquid chromatography (HPLC) analytical trace for the polynorbornene dicarboxyimide monomer comprising SEQ ID NO: 2 (RQDIDLGVSRR).

FIG. 18D depicts the polymerization reaction of polynorbornene dicarboxyimide-based SEQ ID NO: 2 (RQDIDLGVSRR) peptide monomer prepared according to Example 10, and the ¹H NMR spectra for the time course experiments monitoring the polymerization reaction. The disappearance of the resonance at 6=6.5 ppm corresponding to the olefin protons of the monomer (Olefin A) and the coincident appearance of resonances 6=5-6 ppm, which correspond to the cis and trans olefin protons of the polymer backbone (Olefins B).

FIG. 18E shows the kinetics of the polymerization reaction of polynorbornene dicarboxyimide-based SEQ ID NO: 2 (RQDIDLGVSRR) peptide monomer, as determined by the ¹H NMR time course experiment depicted in FIG. 18D.

FIG. 18F shows the SDS-PAGE results for the 11mer of the polynorbornene dicarboxyimide-based brush polymer with SEQ ID NO: 2 (RQDIDLGVSRR) (“DLG”), prepared in Example 10.

FIG. 18G shows the multi-angle light scattering size exclusion chromatography (SEC-MALS) of the polynorbornene dicarboxyimide-based brush homopolymer with SEQ ID NO: 2 (RQDIDLGVSRR) (“DLG”), prepared in Example 10.

FIGS. 19A and 19B show the kinetics for the DLG block (FIG. 19A) and the ETGE block (FIG. 19B), as measured by nuclear magnetic resonance spectroscopy, for the polynorbornene dicarboxyimide-based brush 8:8 copolymer, prepared in Example 11.

FIGS. 19C and 19D show the kinetics for the DLG block (FIG. 19C) and the ETGE block (FIG. 20D), as measured by nuclear magnetic resonance spectroscopy, for the polynorbornene dicarboxyimide-based brush 5:10 copolymer, prepared in Example 11.

FIGS. 19E and 19F show the kinetics for the DLG block (FIG. 19E) and the ETGE block (FIG. 19F), as measured by nuclear magnetic resonance spectroscopy, for the polynorbornene dicarboxyimide-based brush 10:5 copolymer, prepared in Example 11.

FIG. 19G shows the SDS-PAGE results for the polynorbornene dicarboxyimide-based brush copolymers, prepared in Example 11.

FIG. 19H shows the differential refractive index and light scattering for the polynorbornene dicarboxyimide-based brush 8:8 copolymer, prepared in Example 11.

FIG. 19I shows the differential refractive index and light scattering for the polynorbornene dicarboxyimide-based brush 5:10 copolymer, prepared in Example 11.

FIG. 19J shows the differential refractive index and light scattering for the polynorbornene dicarboxyimide-based brush 10:5 copolymer, prepared in Example 11.

FIG. 20 shows the results of an MTS cell viability assay for an Antioxidant Response Element (ARE) Luciferase cell line treated with (a) a tBHQ positive control (triangle) and (b) a polynorbornene dicarboxyimide-based SEQ ID NO: 2 (RQDIDLGVSRR) polymer (i.e., DLG Homopolymer) (circles), as described in Example 12.

FIG. 21A shows the relative luminescence (%) for a polynorbornene dicarboxyimide-based brush homopolymer with SEQ ID NO: 150 (LDPETGEFLRRRR) (“ETGE”), a polynorbornene dicarboxyimide-based brush homopolymer with SEQ ID NO: 2 (RQDIDLGVSRR) (“DLG”), and a polynorbornene dicarboxyimide-based brush copolymer (“DLG:ETGE”), as described in Example 13.

FIG. 21B shows the relative viability (%) for a polynorbornene dicarboxyimide-based brush homopolymer with SEQ ID NO: 150 (LDPETGEFLRRRR) (“ETGE”), a polynorbornene dicarboxyimide-based brush homopolymer with SEQ ID NO: 2 (RQDIDLGVSRR) (“DLG”), and a polynorbornene dicarboxyimide-based brush copolymer (“DLG:ETGE”), as described in Example 13.

FIG. 22 shows a fluorescence polarization Keap1 binding assay, as measured by percent FAM-Nrf2 binding, exhibited by a polynorbornene dicarboxyimide-based brush homopolymer with SEQ ID NO: 150 (LDPETGEFLRRRR) (“ETGE”) and a polynorbornene dicarboxyimide-based brush homopolymer with SEQ ID NO: 2 (RQDIDLGVSRR) (“DLG”), as described in Example 14.

FIG. 23 shows the time-resolved fluorescence resonance energy transfer (TR-FRET) assay for a polynorbornene dicarboxyimide-based brush homopolymer with SEQ ID NO: 150 (LDPETGEFLRRRR) (“ETGE”) and a polynorbornene dicarboxyimide-based brush copolymer (“DLG:ETGE”), as described in Example 15.

FIG. 24 shows a fluorescence polarization Keap1 binding assay, as measured by percent FAM-Nrf2 binding, exhibited by a polynorbornene dicarboxyimide-based brush homopolymer (degree of polymerization=15) with SEQ ID NO: 150 (LDPETGEFLRRRR) (“ETGE”) and polynorbornene dicarboxyimide-based brush copolymers of SEQ ID NO: 150 (LDPETGEFLRRRR) (“ETGE”) with SEQ ID NO: 2 (RQDIDLGVSRR) (“DLG”) at ratios of DLG:ETGE of 5:10, 8:8; and 10:5, as described in Example 16.

FIG. 25A shows a methacrylamide monomer comprising SEQ ID NO: 2 (RQDIDLGVSRR) for use in a reversible addition-fragmentation chain-transfer (RAFT) polymerization, as described in Example 17.

FIG. 25B shows the high-performance liquid chromatography (HPLC) analytical trace for the methacrylamide monomer comprising SEQ ID NO: 2 (RQDIDLGVSRR).

FIG. 25C shows the mass spectrum for the methacrylamide monomer comprising SEQ ID NO: 2 (RQDIDLGVSRR) as measured by electrospray ionization (ESI) mass spectrometry.

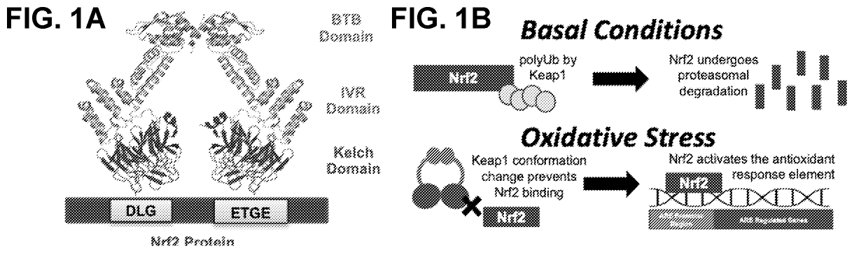

### STATEMENTS REGARDING CHEMICAL COMPOUNDS AND NOMENCLATURE

The following abbreviations are used herein: Keap1 refers to Kelch-like ECH-associated protein 1; Nrf2 refers to Nuclear factor-erythroid factor 2-related factor 2; CNS refers to central nervous system; SPPS refers to solid phase peptide synthesis; ROMP refers to ring-opening metathesis polymerization; RAFT refers to reversible addition fragmentation chain transfer polymerization; DMF refers to dimethylformamide; TFA refers to trifluoroacetic acid; TIPS refers to triisopropyl silane; DTT refers to dithiothreitol; RP-HPLC refers to reverse-phase high performance liquid chromatography; ESI-MS refers to electrospray ionization mass spectrometry; NMR refers to nuclear magnetic resonance spectrometry; MALDI-MS refers to matrix-assisted laser desorption/ionization mass spectrometry; SEC-MALS refers to size-exclusion chromatography coupled with multiangle light scattering; GPC refers to gel permeation chromatography; SDS-PAGE refers to sodium dodecyl sulfate-polyacrylamide gel electrophoresis; ARE refers to antioxidant response element; tBHQ refers to tert-Butylhydroquinone; PLP refers to protein-like polymer; PDI refers to polydispersity index; MW refers to molecular weight; and DP refers to degree of polymerization.

In an embodiment, a peptide, a polymer, or a composition (e.g., formulation) of the invention is isolated or purified. In an embodiment, an isolated or purified peptide, polymer, or composition (e.g., formulation) is at least partially isolated or purified as would be understood in the art. In an embodiment, the peptide, polymer, or composition (e.g., formulation) of the invention has a chemical purity of at least 95%, optionally for some applications at least 99%, optionally for some applications at least 99.9%, optionally for some applications at least 99.99%, and optionally for some applications at least 99.999% pure. The invention includes isolated and purified compositions of any of the brush polymers (e.g., brush homopolymers and peptide brush copolymers) described herein including the brush block polymers or brush random polymers having one or more side chains comprising the peptide analogues, derivative, variants or fragments.

As used herein, the term “polymer” refers to a molecule composed of repeating structural units connected by covalent chemical bonds often characterized by a substantial number of repeating units (e.g., equal to or greater than 3 repeating units, optionally, in some embodiments equal to or greater than 5 repeating units, in some embodiments greater or equal to 10 repeating units) and a high molecular weight (e.g., greater than or equal to 1 kDa, in some embodiments greater than or equal to 5 kDa or greater than or equal to 50 kDa). Polymers are commonly the polymerization product of one or more monomer precursors. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits (e.g., 3 or more monomer subunits, 4 or more monomer subunits, 5 or more monomer subunits, or 6 or more monomer subunits), and include random, block, brush, brush block, alternating, segmented, grafted, tapered and other architectures. In some embodiments, copolymers of the invention comprise from 2 to 10 different monomer subunits. Useful polymers include organic polymers that may be in amorphous, semi-amorphous, crystalline or semi-crystalline states. Cross linked polymers having linked monomer chains are useful for some applications, for example linked by one or more disulfide linkages. The invention provides polymers comprising therapeutic agents, such as brush polymers having at least a portion of the repeating units comprising polymer side chains such as peptide side chains.

As used herein, the term “polymer segment” (e.g., first polymer segment, second polymer segment, etc.) refers to a section (e.g., portion) of the polymer comprising a particular monomer or arrangement of monomers. A polymer segment can be a homopolymer or a copolymer. In embodiments where a polymer segment is a copolymer, the copolymer can exist in any suitable arrangement of monomers (e.g., random, block, brush, brush block, alternating, segmented, grafted, tapered, statistical and other architectures). In some embodiments, the polymer segments are homopolymers, random copolymers, statistical copolymers or block copolymers. Any polymer (e.g., brush polymer) described herein can have a single polymer segment or multiple polymer segments. In embodiments where the polymer has multiple polymer segments, the polymer segments can exist in any suitable arrangement (random, block, brush, brush block, alternating, segmented, grafted, tapered, statistical, and other architectures).

An “oligomer” refers to a molecule composed of repeating structural units connected by covalent chemical bonds often characterized by a number of repeating units less than that of a polymer (e.g., equal to or less than 3 repeating units) and a lower molecular weights (e.g., less than or equal to 1,000 Da) than polymers. Oligomers may be the polymerization product of one or more monomer precursors.

A “peptide” or “oligopeptide” herein are used interchangeably and refer to a polymer of repeating structural units connected by a peptide bond. Typically, the repeating structural units of the peptide are amino acids including naturally occurring amino acids, non-naturally occurring amino acids, analogues of amino acids or any combination of these. The number of repeating structural units of a peptide, as understood in the art, are typically less than a “protein”, and thus the peptide often has a lower molecular weight than a protein.

“Block copolymers” are a type of copolymer comprising blocks or spatially segregated domains, wherein different domains comprise different polymerized monomers, for example, including at least two chemically distinguishable blocks. Block copolymers may further comprise one or more other structural domains, such as hydrophobic groups, hydrophilic groups, etc. In a block copolymer, adjacent blocks are constitutionally different, i.e., adjacent blocks comprise constitutional units derived from different species of monomer or from the same species of monomer but with a different composition or sequence distribution of constitutional units. Different blocks (or domains) of a block copolymer may reside on different ends or the interior of a polymer (e.g., [A][B]), or may be provided in a selected sequence ([A][B][A][B]). “Diblock copolymer” refers to block copolymer having two different polymer blocks. “Triblock copolymer” refers to a block copolymer having three different polymer blocks, including compositions in which two non-adjacent blocks are the same or similar. “Pentablock” copolymer refers to a copolymer having five different polymer including compositions in which two or more non-adjacent blocks are the same or similar.

“Random copolymers” are a type of copolymer comprising spatially randomized units, wherein at least two chemically distinguishable polymerized monomers are randomly distributed throughout the polymer.

“Polymer backbone group” refers to groups that are covalently linked to make up a backbone of a polymer, such as a block copolymer or a random copolymer. Polymer backbone groups may be linked to side chain groups, such as polymer side chain groups. Some polymer backbone groups useful in the present compositions are derived from polymerization of a monomer selected from the group consisting of a substituted or unsubstituted norbornene, olefin, cyclic olefin, norbornene anhydride, cyclooctene, cyclopentadiene, styrene, acrylamide, and acrylate. Some polymer backbone groups useful in the present compositions are obtained from a ring opening metathesis polymerization (ROMP) reaction. Polymer backbones may terminate in a range of backbone terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen, C₁-C₁₀ alkyl or C₅-C₁₀ aryl.

“Polymer side chain group” refers to a group covalently linked (directly or indirectly) to a polymer backbone group that comprises a polymer side chain, optionally imparting steric properties to the polymer. In an embodiment, for example, a polymer side chain group is characterized by a plurality of repeating units having the same, or similar, chemical composition. A polymer side chain group may be directly or indirectly linked to the polymer back bone groups. In some embodiments, polymer side chain groups provide steric bulk and/or interactions that result in an extended polymer backbone and/or a rigid polymer backbone. Some polymer side chain groups useful in the present compositions include unsubstituted or substituted peptide groups. Some polymer side chain groups useful in the present compositions comprise repeating units obtained via anionic polymerization, cationic polymerization, free radical polymerization, group transfer polymerization, or ring-opening polymerization. A polymer side chain may terminate in a wide range of polymer side chain terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen or C₁-C₅ alkyl.

As used herein, the term “degree of polymerization” refers to the average number of monomer units per polymer chain. For example, for certain polymers described herein, comprising Z¹, Z², and/or S monomer units, the degree of polymerization would be represented by the sum total of Z¹, Z², and S monomer units. Since the degree of polymerization can vary from polymer to polymer, the degree of polymerization is generally represented by an average.

As used herein, the term “brush polymer” refers to a polymer comprising repeating units each independently comprising a polymer backbone group covalently linked to at least one polymer side chain group. A brush polymer may be characterized by brush density which refers to the percentage of the repeating units comprising polymer side chain groups. Brush polymers of certain aspects are characterized by a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a brush density selected from the range 50% to 100%, optionally some embodiments a density selected from the range of 75% to 100%, or optionally for some embodiments a density selected from the range of 90% to 100%.

As used herein, the term “peptide density” refers to the percentage of monomer units in the polymer chain which have a peptide covalently linked thereto. The percentage is based on the overall sum of monomer units in the polymer chain. For example, for certain polymers described herein, each P¹ is the polymer side chain comprising the peptide, each P² is a polymer side chain having a composition different from that of P¹, and each S is independently a repeating unit having a composition different from P¹ and P². Thus, the peptide density, or percentage of monomer units comprising the peptide (i.e., P¹ for this particular example) would be represented by the formula:

$$\frac{{P}^{1}}{{P}^{1} + {P}^{2} + S} \times 100 ,$$

where each variable refers to the number of monomer units of that type in the polymer chain. Polymers of certain aspects are characterized by a peptide density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Polymers of certain aspects are characterized by a peptide density selected from the range 50% to 100%, optionally some embodiments a density selected from the range of 75% to 100%, or optionally for some embodiments a density selected from the range of 90% to 100%. In some embodiments, the brush density is equal to the peptide density.

In an aspect, the polymer side chain groups can have any suitable spacing on the polymer backbone. Typically, the space between adjacent polymer side chain groups is from 3 angstroms to 30 angstroms, and optionally 5 to 20 angstroms and optionally 5 to 10 angstroms. By way of illustration, in certain embodiments having a brush density of 100%, the polymer side chain groups typically are spaced 6±5 angstroms apart on the polymer backbone. In some embodiments the brush polymer has a high a brush density (e.g., greater than 70%), wherein the polymer side chain groups are spaced 5 to 20 angstroms apart on the polymer backbone.

The term “sequence homology” or “sequence identity” means the proportion of amino acid matches between two amino acid sequences. When sequence homology is expressed as a percentage, e.g., 50%, the percentage denotes the fraction of matches over the length of sequence that is compared to some other sequence. Gaps (in either of the two sequences) are permitted to maximize matching; for example, wherein gap lengths of 5 amino acids or less, optionally 3 amino acids or less, are usually used. In other words, a sequence having 75% or greater sequence identity of SEQ ID NO: 1 (RQDIDLGVSR) can indicate that the foregoing sequence can have one or two point mutations (i.e., amino acid change), one or two amino acid deletions, one or two amino acid additions, one point mutation and one amino acid deletion, or one point mutation and one amino acid addition. Similarly, a sequence having 85% or greater sequence identity of SEQ ID NO: 1 (RQDIDLGVSR) indicates that the foregoing sequence can have one point mutation (i.e., amino acid change), one amino acid deletion, or one amino acid addition.

The term “fragment” refers to a portion, but not all of, a composition or material, such as a peptide composition or material. In an embodiment, a fragment of a peptide refers to 50% or more of the sequence of amino acids, optionally 70% or more of the sequence of amino acids and optionally 90% or more of the sequence of amino acids.

As used herein, the phrase “charge modulating domain” refers to one or more amino acids added to the peptide sequences described herein to modulate the charge of the peptide. For example, the charge modulating domain can be a TAT sequence, a glycine-serine domain, a cationic residue domain, or a combination thereof, or optionally a glycine-serine domain, a cationic residue domain, or a combination thereof. In certain embodiments, the charge modulating domain has from 1 to 7 amino acid residues (e.g., 2 to 7 amino acid residues or 3 to 7 amino acid residues). The 1 to 7 amino acids can be added in a single block containing from 1 to 7 amino acid residues or more than one block containing from 1 to 6 amino acid residues. In preferred embodiments, the charge modulating domain is a cationic residue domain having from 1 to 7 amino acid residues selected from lysine, arginine, histidine, or a combination thereof. Generally, the charge modulating domain modulates the charge of the peptide to have a net positive charge. Without wishing to be bound by any particular theory, it is believed that the net positive charge increases the cellular uptake of the peptide or polymer comprising the peptide. The overall charge of the peptide or polymer comprising the peptide can be determined by any suitable means. For example, the overall charge can be determined by (i) structural analysis of the functional residues on the peptide sequence and their respective pKa, (ii) physical characterization by measuring the zeta potential, and/or (iii) by virtue of the material moving towards a negative pole in an electrophoresis polymer gel. In certain embodiments, the overall charge of the peptide or polymer comprising the peptide is determined by measuring the zeta potential.

“Polymer blend” refers to a mixture comprising at least one polymer, such as a brush polymer, e.g., brush block copolymer or brush random copolymer, and at least one additional component, and optionally more than one additional component. In some embodiments, for example, a polymer blend of the invention comprises a first brush copolymer and one or more addition brush polymers having a composition different than the first brush copolymer. In some embodiments, for example, a polymer blend of the invention further comprises one or more additional brush block copolymers, brush random copolymers, homopolymers, copolymers, block copolymers, random copolymers, brush block copolymers, oligomers, solvent, small molecules (e.g., molecular weight less than 500 Da, optionally less than 100 Da), or any combination of these. Polymer blends useful for some applications comprise a first brush polymer, and one or more additional components comprising polymers, block copolymers, brush polymers, linear block copolymers, random copolymers, homopolymers, or any combinations of these. Polymer blends of the invention include mixture of two, three, four, five and more polymer components.

As used herein, the term “compound” can be used to refer to any of the peptides or polymers described herein. Alternatively, or additionally, the term compound can refer to any of the synthetic precursors, reagents, additives, excipients, etc. used in preparation of or formulation with the peptides or polymers described herein.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

As used herein, the term “substituted” refers to a compound wherein a hydrogen is replaced by another functional group.

Unless otherwise specified, the term “average molecular weight,” refers to number average molecular weight. Number average molecular weight is the defined as the total weight of a sample volume divided by the number of molecules within the sample. As is customary and well known in the art, peak average molecular weight and weight average molecular weight may also be used to characterize the molecular weight of the distribution of polymers within a sample.

As is customary and well known in the art, hydrogen atoms in formulas (FX1a)-(FX6b) are not always explicitly shown, for example, hydrogen atoms bonded to the carbon atoms of aromatic, heteroaromatic, and alicyclic rings are not always explicitly shown in formulas ((FX1a)-(FX6b). The structures provided herein, for example in the context of the description of formulas (FX1a)-(FX6b) and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g., L¹-L²).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g., L¹-L²).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g., L¹-L²).

As used herein, the terms “heteroarylene” and “heteroarylene group” are used synonymously and refer to a divalent group derived from a heteroaryl group as defined herein. The invention includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g., L¹-L²).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g., L¹-L²).

As used herein, the terms “cycloalkenylene” and “cycloalkenylene group” are used synonymously and refer to a divalent group derived from a cycloalkenyl group as defined herein. The invention includes compounds having one or more cycloalkenylene groups. Cycloalkenylene groups in some compounds function as linking and/or spacer groups. Compounds of the invention include substituted and/or unsubstituted C₃-C₂₀ cycloalkenylene, C₃-C₁₀ cycloalkenylene and C₃-C₅ cycloalkenylene groups, for example, as one or more linking groups (e.g., L¹-L²).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g., L¹-L²).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy)ₙ-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides are comprised of two or more amino acids connected via peptide bonds.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—. Compositions of some embodiments of the invention comprise alkyl groups as terminating groups, such as polymer backbone terminating groups and/or polymer side chain terminating groups.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including —CN;
—COOR where R is a hydrogen or an alkyl group or an aryl group and more specifically where R is a methyl, ethyl, propyl, butyl, or phenyl group all of which groups are optionally substituted;
—COR where R is a hydrogen or an alkyl group or an aryl group and more specifically where R is a methyl, ethyl, propyl, butyl, or phenyl group all of which groups are optionally substituted;
—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;
—SR, where R is hydrogen or an alkyl group or an aryl group and more specifically where R is hydrogen, methyl, ethyl, propyl, butyl, or a phenyl group, which are optionally substituted;

—SO₂R, or —SOR where R is an alkyl group or an aryl group and more specifically where R is a methyl, ethyl, propyl, butyl, or phenyl group, all of which are optionally substituted;
—OCOOR where R is an alkyl group or an aryl group;
—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;
—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, which is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or D- or L-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. Isomers include structural isomers and stereoisomers such as enantiomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to a subject, such as a patient in need of treatment; slowing in the rate of degeneration or decline; making the final point of degeneration less debilitating; improving a subject's physical or mental well-being. The treatment or amelioration of symptoms can be based on objective or subjective parameters; including the results of a physical examination, neuropsychiatric exams, and/or a psychiatric evaluation.

An “effective amount” is an amount sufficient to accomplish a stated purpose (e.g., achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g., transcription factor) relative to the absence of the antagonist. An “activity increasing amount,” as used herein, refers to an amount of agonist (activator) required to increase the activity of an enzyme or protein (e.g., transcription factor) relative to the absence of the agonist. A “function disrupting amount,” as used herein, refers to the amount of antagonist (inhibitor) required to disrupt the function of an enzyme or protein (e.g., transcription factor) relative to the absence of the antagonist. A “function increasing amount,” as used herein, refers to the amount of agonist (activator) required to increase the function of an enzyme or protein (e.g., transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g., agonist) interaction means positively affecting (e.g., increasing) the activity or function of the protein.

The term “modulator” refers to a composition that increases or decreases the level of a target molecule or the function of a target molecule.

“Patient” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a mammal. In some embodiments, a patient is a mouse. In some embodiments, a patient is an experimental animal. In some embodiments, a patient is a rat. In some embodiments, a patient is a test animal.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent or chemotherapeutic). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J Hosp. Pharm. 46:1576-1587, 1989).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der waal's bond(s)/interactions, hydrogen bond(s), polar bond(s), or combinations or mixtures thereof).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

### DETAILED DESCRIPTION OF THE INVENTION

In the following description, numerous specific details of the compounds, compositions components and methods of the present invention are set forth in order to provide a thorough explanation of the precise nature of the invention. It will be apparent, however, to those of skill in the art that the invention can be practiced without these specific details.

In an aspect, the invention provides a peptide having a sequence having 75% or greater sequence (e.g., 80% or greater, 85% or greater, 90% or greater, 95% or greater, or 100%) identity with a 6 to 10 amino acid fragment of SEQ ID NO: 218 (MDLIDILWRQDIDLGVSREVFDFS), wherein the peptide further comprises a charge modulating domain having from 1 to 7 amino acid residues. In certain embodiments, the peptide comprises a sequence having 85% or greater (e.g., 90% or greater, 95% or greater, or 100%) sequence identity with a 6 to 10 amino acid fragment of SEQ ID NO: 218 (MDLIDILWRQDIDLGVSREVFDFS). In preferred embodiments, the peptide comprises a 6 to 10 amino acid fragment of SEQ ID NO: 218 (MDLIDILWRQDIDLGVSREVFDFS).

In some embodiments, the invention provides a peptide having from 11 to 17 amino acid residues (e.g., 11 amino acid residues, 12 amino acid residues, 13 amino acid residues, 14 amino acid residues, 15 amino acid residues, 16 amino acid residues, or 17 amino acid residues) comprising a sequence having 85% or greater sequence identity of SEQ ID NO: 1 (RQDIDLGVSR), SEQ ID NO: 220 (ILWRQDIDLGVSR), or SEQ ID NO: 222 (LWRQDIDLGVSR), wherein the peptide further comprises a charge modulating domain having from 1 to 7 amino acid residues. In some embodiments, the peptide has 11 to 16 amino acid residues, the peptide has 11 to 15 amino acid residues, or the peptide has 11 to 14 amino acid residues. In certain embodiments, the peptide comprises a sequence having 85% or greater sequence identity of SEQ ID NO: 1 (RQDIDLGVSR). In preferred embodiments, the peptide comprises SEQ ID NO: 1 (RQDIDLGVSR).

As used herein, a sequence having 85% or greater sequence identity of SEQ ID NO: 1 (RQDIDLGVSR), SEQ ID NO: 220 (ILWRQDIDLGVSR), or SEQ ID NO: 222 (LWRQDIDLGVSR) indicates that the foregoing sequences can have one point mutation (i.e., amino acid change), one amino acid deletion, or one amino acid addition. In some embodiments, the sequence having 85% or greater sequence identity of SEQ ID NO: 1 (RQDIDLGVSR), SEQ ID NO: 220 (ILWRQDIDLGVSR), or SEQ ID NO: 222 (LWRQDIDLGVSR) is SEQ ID NO: 1 (RQDIDLGVSR), SEQ ID NO: 220 (ILWRQDIDLGVSR), or SEQ ID NO: 222 (LWRQDIDLGVSR).

In this aspect of the invention, the peptide comprises a charge modulating domain having from 1 to 7 amino acid residues. Typically, the charge modulating domain is a glycine-serine domain, a cationic residue domain, or a combination thereof. In certain embodiments, the charge modulating domain is a cationic residue domain having from 1 to 7 amino acid residues selected from lysine, arginine, histidine, and a combination thereof. In preferred embodiments, the charge modulating domain modulates the peptide to have a net positive charge. Without wishing to be bound by any particular theory, it is believed that the net positive charge increases the cellular uptake of the peptide or polymer comprising the peptide. Additionally, the addition of residues to form a net positive charge may enhance the aqueous solubility of the compound to facilitate therapeutic use.

In some embodiments, the peptide having from 11 to 17 amino acid residues comprises a sequence having 75% or greater (e.g., 80% or greater, 85% or greater, 90% or greater, 95% or greater, or 100%) sequence identity of SEQ ID NO: 1-SEQ ID: 76. In some embodiments, the peptide having from 11 to 17 amino acid residues is selected from SEQ ID NO: 1-SEQ ID NO: 76. In certain embodiments, the peptide having from 11 to 17 amino acid residues is selected from SEQ ID NO: 2-SEQ ID NO: 76.

In another aspect, the invention provides a polymer comprising: a first polymer segment comprising at least 2 first repeating units; wherein each of the first repeating units of the first polymer segment comprises a first polymer backbone group directly or indirectly covalently linked to a first polymer side chain group comprising a peptide; wherein the peptide comprises a sequence having 75% or greater (e.g., 80% or greater, 85% or greater, 90% or greater, 95% or greater, or 100%) sequence identity with a 6 to 10 amino acid fragment of SEQ ID NO: 218 (MDLIDILWRQDIDLGVSREVFDFS). In some embodiments, the peptide comprises a sequence having 85% or greater (e.g., 90% or greater, 95% or greater, or 100%) sequence identity with a 6 to 10 amino acid fragment of SEQ ID NO: 218 (MDLIDILWRQDIDLGVSREVFDFS). In certain embodiments, the peptide comprises a 6 to 10 amino acid fragment of SEQ ID NO: 218 (MDLIDILWRQDIDLGVSREVFDFS).

In some embodiments, the invention provides a polymer comprising a first polymer segment comprising at least 2 first repeating units; wherein each of the first repeating units of the first polymer s comprises a first polymer backbone group directly or indirectly covalently linked to a first polymer side chain group comprising a peptide; wherein the peptide comprises a sequence having 75% or greater (e.g., 80% or greater, 85% or greater, 90% or greater, 95% or greater, or 100%) sequence identity of SEQ ID NO: 1 (RQDIDLGVSR), SEQ ID NO: 220 (ILWRQDIDLGVSR), or SEQ ID NO: 222 (LWRQDIDLGVSR). The inventive polymer can be any suitable polymer type described herein and can comprise, or be derived from, any suitable number of monomers. For example, in some embodiments, the polymer is a homopolymer (i.e., derived from one type of monomer). Alternatively, in some embodiments, the polymer can be a copolymer comprising (e.g., derived from) more than one type of monomer (e.g., from 2 to 10 types of monomers). It will be understood that the inventive polymer, along with the linked polymer side chains, can have any suitable configuration. For example, in some embodiments wherein the polymer is a homopolymer, the polymer can be a brush polymer. In other embodiments wherein the polymer is a copolymer, the polymer can be a brush block copolymer or brush random copolymer.

The polymer comprises a first polymer segment comprising at least 2 first repeating units, and optionally at least 5 first repeating units (e.g., 2-30, 5-30, 10-30, 15-30, or 20-30 first repeating units); wherein each of the first repeating units of the first polymer segment comprises a first polymer backbone group directly or indirectly covalently linked to a first polymer side chain group comprising a peptide (e.g., a therapeutic peptide) comprising a sequence having 75% or greater sequence (e.g., 80% or greater, 85% or greater, 90% or greater, 95% or greater, or 100%) identity of SEQ ID NO: 1 (RQDIDLGVSR), SEQ ID NO: 220 (ILWRQDIDLGVSR), or SEQ ID NO: 222 (LWRQDIDLGVSR).

Thus, at least one polymer side chain (e.g., the first polymer segment) comprises a therapeutic peptide (i.e., peptide). The peptide comprises any suitable number of amino acid units so long as the peptide comprises a sequence having 75% or greater (e.g., 80% or greater, 85% or greater, 90% or greater, 95% or greater, or 100%) sequence identity of SEQ ID NO: 1 (RQDIDLGVSR), SEQ ID NO: 220 (ILWRQDIDLGVSR), or SEQ ID NO: 222 (LWRQDIDLGVSR). In keeping with an aspect of the invention, the therapeutic peptide comprises at least 10 amino acid units. For example, the peptide comprises 10 or more amino acid units, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more amino acid units. Alternatively, or in addition, the peptide can comprise 100 or less amino acid units, for example, 90 or less, 80 or less, 70 or less, 60 or less, 59 or less, 58 or less, 57 or less, 56 or less, 55 or less, 54 or less, 53 or less, 52 or less, 51 or less, 50 or less, 49 or less, 48 or less, 47 or less, 46 or less, 45 or less, 44 or less, 43 or less, 42 or less, 41 or less, 40 or less, 39 or less, 38 or less, 37 or less, 36 or less, 35 or less, 34 or less 33 or less, 32 or less, or 31 or less amino acid units. Thus, the peptide can comprise a number of amino acid units bounded by any two of the aforementioned endpoints. For example, the peptide can comprise 10 to 100 amino acid units, for example, 10 to 90, 10 to 80, 10 to 70, 10 to 60, 10 to 50, 10 to 40, 10 to 30, 10 to 20, 10 to 16, 11 to 100, 11 to 90, 11 to 80, 11 to 70, 11 to 60, 11 to 50, 11 to 40, 11 to 30, 11 to 20, 11 to 16, 12 to 100, 12 to 90, 12 to 80, 12 to 70, 12 to 60, 12 to 50, 12 to 40, 12 to 30, 12 to 20, 12 to 16, 11 to 17, 11 to 16, 11 to 15, or 11 to 14 amino acid units. In some embodiments, the peptide comprises 11 to 17 amino acids. In certain embodiments, the peptide comprises 11 to 16 amino acid, 11 to 15 amino acids, or 11 to 14 amino acids.

The peptide can have any suitable structure (e.g., primary, secondary, tertiary, or quaternary structure) described herein. The peptide can be a branched peptide, a linear peptide, cyclic peptide, or a cross-linked peptide. In some embodiments, the polymer is characterized by a structure wherein at least a portion of the peptide is linked to the polymer backbone group via an enzymatically degradable linker, such a matrix metalloproteinase (MMP) cleavage sequence, cathepsin B cleavage sequence, ester bond, reductive sensitive bond-disulfide bond, pH sensitive bond-imine bond or any combinations of these. In other embodiments, the polymer is characterized by a structure wherein at least a portion of the peptide side-chain is linked to the polymer backbone or consists of a degradable or triggerable linker. In some embodiments, the peptide and/or polymer further comprises a tag for imaging and/or analysis. For example, the peptide and/or polymer can further comprise a dye, radiolabeling, an imaging agent, tritiation, and the like.

In some embodiments, the peptide comprising a sequence having 75% or greater (e.g., 80% or greater, 85% or greater, 90% or greater, 95% or greater, or 100%) sequence identity of SEQ ID NO: 1 (RQDIDLGVSR), SEQ ID NO: 220 (ILWRQDIDLGVSR), or SEQ ID NO: 222 (LWRQDIDLGVSR) further comprises a charge modulating domain. The charge modulating domain can be any suitable amino acid domain, which increases the positive charge of the peptide. For example, the charge modulating domain can be a TAT sequence, a glycine-serine domain, a cationic residue domain, or a combination thereof. In some embodiments, the charge modulating domain is a glycine-serine domain, a cationic residue domain, or a combination thereof. In certain embodiments, the charge modulating domain is a cationic residue domain having from 1 to 7 amino acid residues selected from lysine, arginine, histidine, and a combination thereof. In preferred embodiments, the charge modulating domain modulates the peptide to have a net positive charge.

In some embodiments, the peptide comprises a sequence having 85% or greater sequence identity of SEQ ID NO: SEQ ID NO: 1 (RQDIDLGVSR), SEQ ID NO: 220 (ILWRQDIDLGVSR), or SEQ ID NO: 222 (LWRQDIDLGVSR). In preferred embodiments, the peptide comprises SEQ ID NO: 1 (RQDIDLGVSR), SEQ ID NO: 220 (ILWRQDIDLGVSR), or SEQ ID NO: 222 (LWRQDIDLGVSR). In certain embodiments, the peptide comprises a sequence having 85% or greater sequence identity of SEQ ID NO: 1 (RQDIDLGVSR). In preferred embodiments, the peptide comprises SEQ ID NO: 1 (RQDIDLGVSR). In an embodiment, the peptide is selected from SEQ ID NO: 1-SEQ ID NO: 76, optionally wherein the peptide is selected from SEQ ID NO: 2-SEQ ID NO: 76. In certain embodiments, the peptide is SEQ ID NO: 2 (RQDIDLGVSRR)

In some embodiments, the polymer is characterized by the formula (FX1a), (FX1b), (FX1c), (FX1d); (FX1e); (FX1f); or (FX1g):

Q¹-T-Q² (FX1a);
Q¹-T-[S]ₕ-Q² (FX1b);
Q¹-[S]ₕ-T-Q² (FX1c);
Q¹-[S]ᵢ-T-[S]ₕ-Q² (FX1d);
Q¹-[S]ᵢ-T-[S]ₕ-T-Q² (FX1e);
Q¹-T-[S]ᵢ-T-[S]ₕ-Q² (FX1f);
or
Q¹-T-[S]ᵢ-T-[S]ₕ-T-Q² (FX1g);

wherein each T is independently the first polymer segment comprising the first repeating units and each S is independently an additional polymer segment; Q¹ is a first backbone terminating group; Q² is a second backbone terminating group; and wherein h is zero or an integer selected over the range of 1 to 1000 (e.g., 1 to 500, 1 to 250, 1 to 100, or 1 to 50) and i is zero or an integer selected over the range of 1 to 1000 (e.g., 1 to 500, 1 to 250, 1 to 100, or 1 to 50). In an embodiment, the polymer is characterized by any of formulas (FX1a)-(FX1g), wherein each -T- is independently —[Y¹]ₘ—; wherein each Y¹ is independently the first repeating unit of the first polymer segment; and each m is independently an integer selected from the range 0 to 1000 (e.g., 0 or 1 to 500, 1 to 250, 1 to 100, or 1 to 50), provided that at least one m is an integer selected from the range 1 to 1000 (e.g., 1 to 500, 1 to 250, 1 to 100, or 1 to 50). In certain embodiments, each of the first polymer segment backbone group and/or the additional polymer segment backbone group is a substituted or unsubstituted polymerized norbornene, olefin, cyclic olefin, norbornene anhydride, cyclooctene, cyclopentadiene, styrene, acrylamide, or acrylate.

In certain embodiments, the polymer is characterized by the formula (FX2a), (FX2b), or (FX2c):

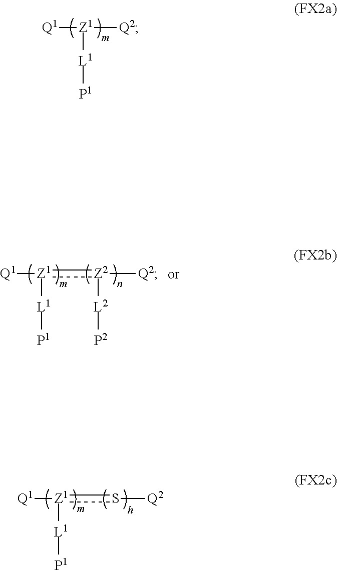

wherein each Z¹ is independently a first polymer backbone group and each Z² is independently a second polymer backbone group; each S is independently a repeating unit having a composition different from the first repeating unit; Q¹ is a first backbone terminating group and Q² is a second backbone terminating group; each L¹ is independently a first linking group, each L² is independently a second linking group; each P¹ is the peptide; wherein each P² is a polymer side chain having a composition different from that of P¹; wherein each P² is a polymer side chain having a composition different from that of P¹; and wherein each m is independently an integer selected from the range of 2 to 1000 (e.g., 2 to 500, 2 to 250, 2 to 100, or 2 to 50); wherein each n is each independently an integer selected from the range of 0 to 1000 (e.g., 0 to 500, 0 to 250, 0 to 100, or 0 to 50); and wherein h are each independently an integer selected from the range of 0 to 1000 (e.g., 0 to 500, 0 to 250, 0 to 100, or 0 to 50). In certain embodiments, each of the first polymer backbone group and/or the second polymer backbone group is a substituted or unsubstituted polymerized norbornene, olefin, cyclic olefin, norbornene anhydride, cyclooctene, cyclopentadiene, styrene, acrylamide, or acrylate.

For each of the polymers characterized by the formula (FX2a), (FX2b), or (FX2c), described herein, it will be understood that the first polymer backbone group units, the second polymer backbone group units, and the repeating unit having a composition different from the first repeating unit can be arranged in any suitable order. For example, the first polymer backbone group units, the second polymer backbone group units, and the repeating unit having a composition different from the first repeating unit can be arranged as a random polymer, block polymer, brush, brush block, alternating, segmented, grafted, tapered and other architectures. In other words, variables “m”, “n”, and “h” merely define the total number of that particular monomer in the polymer and do not imply any particular order.

In certain embodiments, for each of the polymers characterized by the formula (FX2a), (FX2b), and (FX2c), each of Z¹ and Z² can be any suitable monomer capable of undergoing ring opening metathesis or cross metathesis. For example, each of Z¹ and Z² can independently be a substituted or unsubstituted norbornene, oxanorbornene, olefin, cyclic olefin, cyclooctene, or cyclopentadiene. In some embodiments, each of the first polymer backbone group and/or the second polymer backbone group is a polymerized norbornene dicarboxyimide monomer. In preferred embodiments, each polymer backbone group of the polymer is a polymerized norbornene dicarboxyimide monomer.

Thus, for each of the polymers characterized by the formula (FX2a), (FX2b), and (FX2c), each Z¹ connected to L¹, and P¹ or a combination thereof can independently be characterized by the formula (FX3a) or (FX3b):

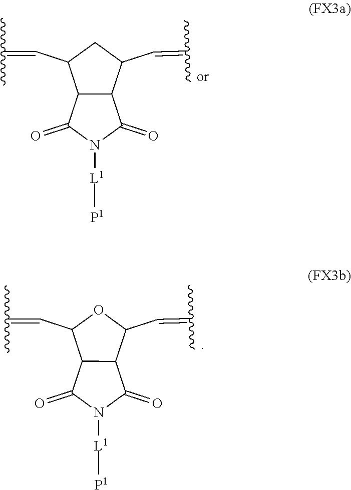

and when present, each Z² connected to L², and P² or a combination thereof can independently be characterized by the formula (FX4a) or (FX4b)

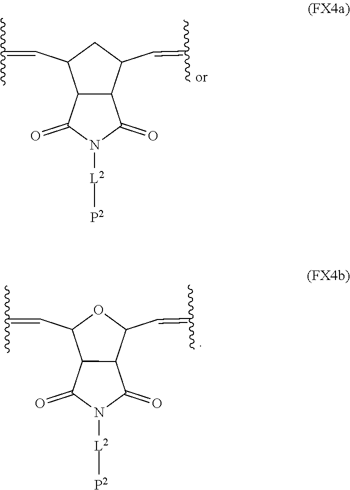

In certain embodiments of the polymers characterized by the formula (FX2a), (FX2b), and (FX2c), each Z¹ connected to L¹, and P¹ or a combination thereof is independently characterized by the formula (FX3a):

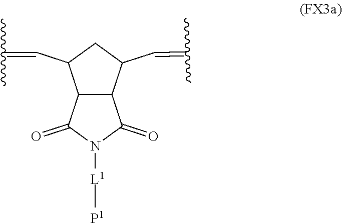

and/or each Z² connected to L², and P² or a combination thereof is independently characterized by the formula (FX4a):

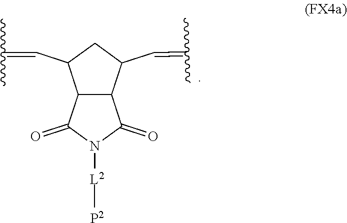

For each of the polymers characterized by the formula (FX1a), (FX1b), (FX1c), (FX1d), (FX1e), (FX1f), (FX1g), (FX2a), (FX2b), and (FX2c), each of Q¹ and Q² can independently be selected from a hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀alkylaryl, —CO₂R³, —CONR⁴R⁵, —COR⁶, —SOR⁷, —OSR⁷, —SO₂R⁹, —OR¹⁰, —SR¹¹, —NR¹²R¹³, NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ is independently H, C₅-C₁₀ aryl or C₁-C₁₀ alkyl.

For each of the polymers characterized by the formula (FX2a), (FX2b), and (FX2c), each of L¹ and L² can be any suitable linking group. For example, each of L¹ and L² can independently be selected from a single bond, an oxygen, and groups having an alkyl group, an alkenylene group, an arylene group, an alkoxy group, an acyl group, a triazole group, a diazole group, a pyrazole group, and combinations thereof. In certain embodiments, each of L¹ and L² is independently selected from a single bond, —O—, C₁-C₁₀ alkyl, C₂-C₁₀ alkenylene, C₃-C₁₀ arylene, C₁-C₁₀ alkoxy, C₁-C₁₀ acyl and combinations thereof.

For each of the polymers characterized by the formula (FX2a), (FX2b), and (FX2c), each P² is a polymer side chain having a composition different from that of P¹. Thus, P² can be any suitable side chain capable of being incorporated into the polymer with P¹. In some embodiments, P² is a peptide or protein other than P¹. Thus, the polymer can comprise two different peptide or protein units. In some embodiments, P² is a nonionic polymer selected from a polyalkylene glycol, a polyetheramine, a polyethylene oxide/polypropylene oxide copolymer, a polysaccharide, and combinations thereof. In certain embodiments, the nonionic polymer is a polyalkylene glycol (e.g., polyethylene glycol (PEG) or polypropylene oxide (PPO)), a polyethylene oxide/polypropylene oxide copolymer, or a combination thereof. In preferred embodiments, the nonionic polymer is a polyethylene glycol (PEG). Thus, in some embodiments, each Z² connected to L², and P² or a combination thereof is independently characterized by the formula (FX7a) or (FX7b):

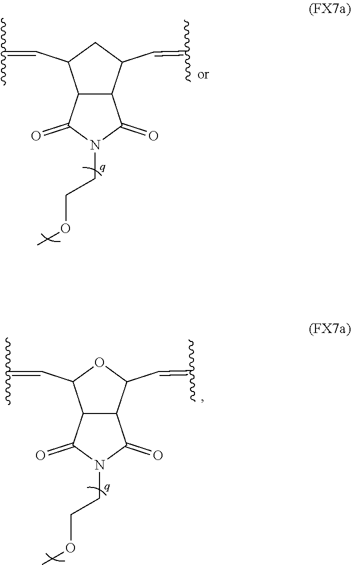

wherein q is an integer from 1 to 500 (e.g., 1 to 250, 1 to 100, 1 to 50, 1 to 25, 1 to 10, or 1 to 6).

For each of the polymers characterized by the formula (FX2a), (FX2b), and (FX2c), each S is independently a repeating unit having a composition different from the first repeating unit. Thus, S can be any monomer unit capable of being incorporated into the polymer with P¹. In some embodiments, S comprises a nonionic polymer selected from a polyalkylene glycol, a polyetheramine, a polyethylene oxide/polypropylene oxide copolymer, a polysaccharide, and combinations thereof. In certain embodiments, the nonionic polymer is a polyalkylene glycol (e.g., polyethylene glycol (PEG) or polypropylene oxide (PPO)), a polyethylene oxide/polypropylene oxide copolymer, or a combination thereof. In preferred embodiments, the nonionic polymer is a polyethylene glycol (PEG). Thus, in some embodiments, each S is independently characterized by the formula (FX7a) or (FX7b):

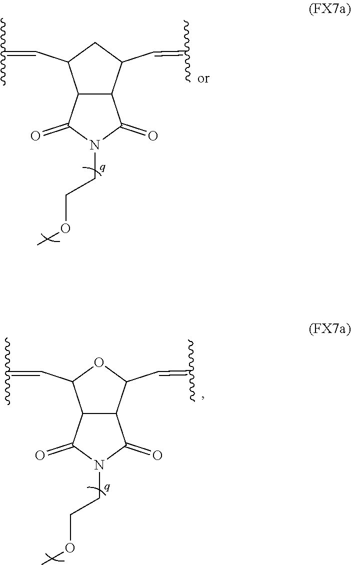

wherein q is an integer from 1 to 500 (e.g., 1 to 250 1 to 100, 1 to 50, 1 to 25, 1 to 10, or 1 to 6)

In certain embodiments, the polymer is characterized by the formula (FX2a), (FX2b), or (FX2c):

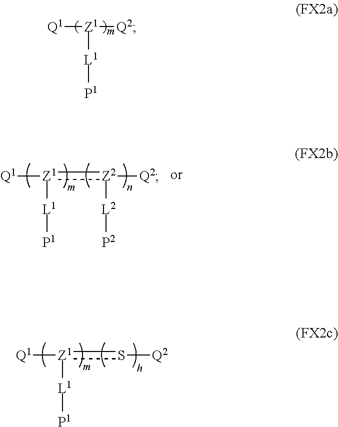

wherein each Z¹ is independently a first polymer backbone group and each Z² is independently a second polymer backbone group; each S is independently a repeating unit having a composition different from the first repeating unit; Q¹ is a first backbone terminating group and Q² is a second backbone terminating group; each L¹ is independently a first linking group, each L² is independently a second linking group; each P¹ is the polymer side chain comprising the peptide; wherein each P² is a polymer side chain having a composition different from that of P¹; each m is independently an integer selected from the range of 2 to 100; each n is independently an integer selected from the range of 0 to 100; and each h is independently an integer selected from the range of 0 to 100, provided that each of the first polymer backbone group and/or the second polymer backbone group is a polymerized norbornene dicarboxyimide monomer, and wherein the peptide comprises a sequence having 75% or greater (e.g., 80% or greater, 85% or greater, 90% or greater, 95% or greater, or 100%) sequence identity of SEQ ID NO: 1 (RQDIDLGVSR), SEQ ID NO: 220 (ILWRQDIDLGVSR), or SEQ ID NO: 222 (LWRQDIDLGVSR). In some embodiments, the polymer further fulfills (i) and/or (ii) of the following properties:
(i) the polymer has a degree of polymerization of 5 to 100, and
(ii) the polymer has a peptide density of greater than 50%, as defined by the following formula:

$$\frac{{P}^{1}}{{P}^{1} + {P}^{2} + S} \times 100.$$

In preferred embodiments, the polymer fulfills both of properties (i) and (ii) above.

In some specific embodiments, the polymer comprises one or more peptides and/or proteins other than the therapeutic peptide described herein (i.e., one or more additional peptides and/or proteins). For example, each polymer segment S of formula (FX1a), (FX1b), (FX1c), (FX1d); (FX1e); (FX1f); or (FX1g) can independently comprise a peptide or protein other than the therapeutic peptide described herein. Similarly, each P² of formula (FX2a), (FX2b), or (FX2c) can independently comprise a peptide or protein other than the therapeutic peptide described herein.

The one or more additional peptides and/or proteins can be any suitable peptide or protein, having any suitable function. For example, the one or more additional peptides and/or proteins can be an additional therapeutic peptide (e.g., an additional therapeutic peptide described herein), a cell-penetrating agent (e.g., a cell-penetrating peptide), a targeting agent (e.g., a target-specific peptide to a tissue or cell type), a therapeutically synergistic disease-specific peptide (e.g. a peptide known or thought to be therapeutic for a disease state, such as but not limited to, neurodegenerative disease), an antibody, or a combination thereof. The additional peptides and/or proteins can be linked to the polymer backbone by any suitable means. In some embodiments, the additional peptides and/or proteins are linked to the polymer backbone via an enzymatically degradable linker (i.e., linking group or linking moiety). Examples of suitable cleavable, degradable or triggerable linkers include enzyme cleavable sequences such as one or more matrix metalloproteinase (MMP) cleavage sequence, cathepsin B cleavage sequence, ester bond, reductive sensitive bond-disulfide bond, pH sensitive bond-imine bond, among others.

The one or more additional peptides and/or proteins can have any suitable number of amino acid units. For example, the one or more additional peptides and/or proteins can comprise 2 or more amino acid units, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more amino acid units. Alternatively, or in addition, the one or more additional peptides and/or proteins can comprise 100 or less amino acid units, for example, 90 or less, 80 or less, 70 or less, 60 or less, 59 or less, 58 or less, 57 or less, 56 or less, 55 or less, 54 or less, 53 or less, 52 or less, 51 or less, 50 or less, 49 or less, 48 or less, 47 or less, 46 or less, 45 or less, 44 or less, 43 or less, 42 or less, 41 or less, 40 or less, 39 or less, 38 or less, 37 or less, 36 or less, 35 or less, 34 or less 33 or less, 32 or less, or 31 or less amino acid units. Thus, the one or more additional peptides and/or proteins can comprise a number of amino acid units bounded by any two of the aforementioned endpoints. For example, the one or more additional peptides and/or proteins can comprise 2 to 100 amino acid units, for example, 2 to 90, 2 to 80, 2 to 70, 2 to 60, 5 to 100, 5 to 90, 5 to 80, 5 to 70, 5 to 60, 3 to 60, 3 to 59, 4 to 58, 5 to 57, 6 to 56, 7 to 55, 8 to 54, 9 to 53, 10 to 52, 11 to 51, 12 to 50, 13 to 49, 14 to 48, 15 to 47, 16 to 46, 17 to 45, 18 to 44, 19 to 43, 20 to 42, 21 to 41, 22 to 42, 23 to 41, 24 to 40, 25 to 39, 26 to 38, 27 to 37, 28 to 36, 29 to 35, 30 to 34, or 31 to 33 amino acid units. In certain embodiments, the one or more additional peptides and/or proteins comprises 5 to 100 amino acids. In preferred embodiments, the one or more additional peptides and/or proteins comprises 8 to 60 amino acid.

The one or more additional peptides and/or proteins can have any suitable structure (e.g., primary, secondary, tertiary, or quaternary structure). Additionally, the one or more additional peptides and/or proteins can be branched, linear, cyclic, or cross-linked. In some embodiments, the one or more additional peptides and/or proteins is a charge modulating domain. For example, the one or more additional peptides and/or proteins can be or can comprise a TAT sequence, a glycine-serine domain, a cationic residue domain, or a combination thereof, or optionally a glycine-serine domain, a cationic residue domain, or a combination thereof. In certain embodiments, the one or more additional peptides and/or proteins modulates the charge of the polymer to have a net positive charge. Without wishing to be bound by any particular theory, it is believed that the net positive charge increases the cellular uptake of the polymer comprising the peptide.

In some embodiments, the polymer further comprises a second polymer segment comprising at least 2 second repeating units and optionally 2-30, 5-30, 10-30, 15-30, or 20-30 second repeating units; wherein each of the second repeating units of the second polymer segment comprises a second polymer backbone group (e.g., Z² described herein) directly or indirectly covalently linked (e.g., L² described herein) to a second polymer side chain group comprising a second peptide (e.g., P² described herein); wherein the second peptide comprises a sequence having 75% or greater (e.g., 80% or greater, 85% or greater, 90% or greater, 95% or greater, or 100%) sequence identity of SEQ ID NO: 77 (LDEETGEFL), optionally wherein the peptide comprises SEQ ID NO: 77 (LDEETGEFL) or SEQ ID NO: 78 (LDPETGEFL). In certain embodiments, the second peptide further comprises a second charge modulating domain having from 2 to 7 amino acid residues and/or has a total of from 11 to 16 amino acid residues (e.g., from 11 to 15 amino acid residues, from 11 to 14 amino acid residues, from 12 to 16 amino acid residues, from 12 to 15 amino acid residues, or from 12 to 14 amino acid residues).

In some embodiments, the second peptide comprises a sequence having 85% or greater sequence identity of SEQ ID NO: 77 (LDEETGEFL). In certain embodiments, the second peptide comprises a sequence having 75% or greater or 85% or greater sequence identity of SEQ ID NO: 77 (LDEETGEFL) has a point mutation to comprise a proline residue and/or a point mutation to delete a glutamate residue. In preferred embodiments, the second peptide comprises SEQ ID NO: 77 (LDEETGEFL) or SEQ ID NO: 78 (LDPETGEFL). In other embodiments, the second peptide comprises SEQ ID NO: 79 (LDPTGEFL) or SEQ ID NO: 80 (LDPETGFL). In other words, a sequence having 75% or greater sequence identity of SEQ ID NO: 77 (LDEETGEFL) or SEQ ID NO: 78 (LDPETGEFL) can indicate that the foregoing sequences can have one or two point mutations (i.e., amino acid change), one or two amino acid deletions, one or two amino acid additions, one point mutation and one amino acid deletion, or one point mutation and one amino acid addition. Similarly, a sequence having 85% or greater sequence identity of SEQ ID NO: 77 (LDEETGEFL) or SEQ ID NO: 78 (LDPETGEFL) indicates that the foregoing sequences can have one point mutation (i.e., amino acid change), one amino acid deletion, or one amino acid addition.

In some embodiments, the second peptide comprises a second charge modulating domain having from 2 to 7 amino acid residues. Typically, the second charge modulating domain is a second glycine-serine domain, a second cationic residue domain, or a combination thereof. In certain embodiments, the second charge modulating domain is a second cationic residue domain having from 2 to 7 amino acid residues selected from lysine, arginine, histidine, and a combination thereof. In preferred embodiments, the second charge modulating domain modulates the second peptide to have a net positive charge. Without wishing to be bound by any particular theory, it is believed that the net positive charge increases the cellular uptake of the second peptide or polymer comprising the second peptide. Additionally, the addition of residues to form a net positive charge may enhance the aqueous solubility of the compound to facilitate therapeutic use.

In some embodiments, the second peptide comprises a sequence having 75% or greater (e.g., 80% or greater, 85% or greater, 90% or greater, 95% or greater, or 100%) sequence identity of SEQ ID NO: 77-SEQ ID: 216. In certain embodiments, the second peptide is selected from: SEQ ID NO: 81 (LDEETGEFLRR), SEQ ID NO: 82 (LDEETGEFLRRR), SEQ ID NO: 83 (LDEETGEFLRRRR), SEQ ID NO: 84 (LDEETGEFLRRRRR), SEQ ID NO: 85 (RRLDEETGEFL), SEQ ID NO: 86 (RRRLDEETGEFL), SEQ ID NO: 87 (RRRRLDEETGEFL), SEQ ID NO: 88 (RRRRRLDEETGEFL), SEQ ID NO: 89 (RRLDEETGEFLRR), SEQ ID NO: 90 (RRRLDEETGEFLRRR), SEQ ID NO: 91 (RLDEETGEFLR), SEQ ID NO: 92 (RLDEETGEFLRR), SEQ ID NO: 93 (RRLDEETGEFLR), SEQ ID NO: 94 (RLDEETGEFLRRR), SEQ ID NO: 95 (RRRLDEETGEFLR), SEQ ID NO: 96 (RLDEETGEFLRRRR), SEQ ID NO: 97 (RRLDEETGEFLRRR), SEQ ID NO: 98 (RRRLDEETGEFLRR), SEQ ID NO: 99 (RRRRLDEETGEFLR), SEQ ID NO: 100 (RLDEETGEFLRRRRR), SEQ ID NO: 101 (RRLDEETGEFLRRRR), SEQ ID NO: 102 (RRRRLDEETGEFLRR), SEQ ID NO: 103 (RRRRRLDEETGEFLR), SEQ ID NO: 104 (LDEETGEFLKK), SEQ ID NO: 105 (LDEETGEFLKKK), SEQ ID NO: 106 (LDEETGEFLKKKK), SEQ ID NO: 107 (LDEETGEFLKKKKK), SEQ ID NO: 108 (KKLDEETGEFL), SEQ ID NO: 109 (KKKLDEETGEFL), SEQ ID NO: 110 (KKKKLDEETGEFL), SEQ ID NO: 111 (KKKKKLDEETGEFL), SEQ ID NO: 112 (KKLDEETGEFLKK), SEQ ID NO: 113 (KKKLDEETGEFLKKK), SEQ ID NO: 114 (KLDEETGEFLK), SEQ ID NO: 115 (KLDEETGEFLKK), SEQ ID NO: 116 (KKLDEETGEFLK), SEQ ID NO: 117 (KLDEETGEFLKKK), SEQ ID NO: 118 (KKKLDEETGEFLK), SEQ ID NO: 119 (KLDEETGEFLKKKK), SEQ ID NO: 120 (KKLDEETGEFLKKK), SEQ ID NO: 121 (KKKLDEETGEFLKK), SEQ ID NO: 122 (KKKKLDEETGEFLK), SEQ ID NO: 123 (KLDEETGEFLKKKKK), SEQ ID NO: 124 (KKLDEETGEFLKKKK), SEQ ID NO: 125 (KKKKLDEETGEFLKK), SEQ ID NO: 126 (KKKKKLDEETGEFLK), SEQ ID NO: 127 (LDEETGEFLKRKR), SEQ ID NO: 128 (KRKRLDEETGEFL), SEQ ID NO: 129 (RKRKLDEETGEFL), SEQ ID NO: 130 (LDEETGEFLRKRK), SEQ ID NO: 131 (KKLDEETGEFLRR), SEQ ID NO: 132 (RRLDEETGEFLKK), SEQ ID NO: 133 (KLDEETGEFLRRR), SEQ ID NO: 134 (KKKLDEETGEFLR), SEQ ID NO: 135 (RRRLDEETGEFLK), SEQ ID NO: 136 (KRLDEETGEFLKR), SEQ ID NO: 137 (RKLDEETGEFLRK), SEQ ID NO: 138 (RKLDEETGEFLKR), SEQ ID NO: 139 (KRLDEETGEFLRK), SEQ ID NO: 140 (LDEETGEFLKKRR), SEQ ID NO: 141 (LDEETGEFLRRKK), SEQ ID NO: 142 (KKRRLDEETGEFL), SEQ ID NO: 143 (RRKKLDEETGEFL), SEQ ID NO: 144 (LDEETGEFLGSGSGRR), SEQ ID NO: 145 (GSGSGRRLDEETGEFL), SEQ ID NO: 146 (LDEETGEFLGSGSGKK), SEQ ID NO: 147 (GSGSGKKLDEETGEFL), SEQ ID NO: 148 (LDPETGEFLRR), SEQ ID NO: 149 (LDPETGEFLRRR), SEQ ID NO: 150 (LDPETGEFLRRRR), SEQ ID NO: 151 (LDPETGEFLRRRRR), SEQ ID NO: 152 (RRLDPETGEFL), SEQ ID NO: 153 (RRRLDPETGEFL), SEQ ID NO: 154 (RRRRLDPETGEFL), SEQ ID NO: 155 (RRRRRLDPETGEFL), SEQ ID NO: 156 (RRLDPETGEFLRR), SEQ ID NO: 157 (RRRLDPETGEFLRRR), SEQ ID NO: 158 (RLDPETGEFLR), SEQ ID NO: 159 (RLDPETGEFLRR), SEQ ID NO: 160 (RRLDPETGEFLR), SEQ ID NO: 161 (RLDPETGEFLRRR), SEQ ID NO: 162 (RRRLDPETGEFLR), SEQ ID NO: 163 (RLDPETGEFLRRRR), SEQ ID NO: 164 (RRLDPETGEFLRRR), SEQ ID NO: 165 (RRRLDPETGEFLRR), SEQ ID NO: 166 (RRRRLDPETGEFLR), SEQ ID NO: 167 (RLDPETGEFLRRRRR), SEQ ID NO: 168 (RRLDPETGEFLRRRR), SEQ ID NO: 169 (RRRRLDPETGEFLRR), SEQ ID NO: 170 (RRRRRLDPETGEFLR), SEQ ID NO: 171 (LDPETGEFLKK), SEQ ID NO: 172 (LDPETGEFLKKK), SEQ ID NO: 173 (LDPETGEFLKKKK), SEQ ID NO: 174 (LDPETGEFLKKKKK), SEQ ID NO: 175 (KKLDPETGEFL), SEQ ID NO: 176 (KKKLDPETGEFL), SEQ ID NO: 177 (KKKKLDPETGEFL), SEQ ID NO: 178 (KKKKKLDPETGEFL), SEQ ID NO: 179 (KKLDPETGEFLKK), SEQ ID NO: 180 (KKKLDPETGEFLKKK), SEQ ID NO: 181 (KLDPETGEFLK), SEQ ID NO: 182 (KLDPETGEFLKK), SEQ ID NO: 183 (KKLDPETGEFLK), SEQ ID NO: 184 (KLDPETGEFLKKK), SEQ ID NO: 185 (KKKLDPETGEFLK), SEQ ID NO: 186 (KLDPETGEFLKKKK), SEQ ID NO: 187 (KKLDPETGEFLKKK), SEQ ID NO: 188 (KKKLDPETGEFLKK), SEQ ID NO: 189 (KKKKLDPETGEFLK), SEQ ID NO: 190 (KLDPETGEFLKKKKK), SEQ ID NO: 191 (KKLDPETGEFLKKKK), SEQ ID NO: 192 (KKKKLDPETGEFLKK), SEQ ID NO: 193 (KKKKKLDPETGEFLK), SEQ ID NO: 194 (LDPETGEFLKRKR), SEQ ID NO: 195 (KRKRLDPETGEFL), SEQ ID NO: 196 (RKRKLDPETGEFL), SEQ ID NO: 197 (LDPETGEFLRKRK), SEQ ID NO: 198 (KKLDPETGEFLRR), SEQ ID NO: 199 (RRLDPETGEFLKK), SEQ ID NO: 200 (KLDPETGEFLRRR), SEQ ID NO: 201 (KKKLDPETGEFLR), SEQ ID NO: 202 (RRRLDPETGEFLK), SEQ ID NO: 203 (KRLDPETGEFLKR), SEQ ID NO: 204 (RKLDPETGEFLRK), SEQ ID NO: 205 (RKLDPETGEFLKR), SEQ ID NO: 206 (KRLDPETGEFLRK), SEQ ID NO: 207 (LDPETGEFLKKRR), SEQ ID NO: 208 (LDPETGEFLRRKK), SEQ ID NO: 209 (KKRRLDPETGEFL), SEQ ID NO: 210 (RRKKLDPETGEFL), SEQ ID NO: 211 (LDPETGEFLGSGSGRR), SEQ ID NO: 212 (GSGSGRRLDPETGEFL), SEQ ID NO: 213 (LDPETGEFLGSGSGKK), SEQ ID NO: 214 (GSGSGKKLDPETGEFL), SEQ ID NO: 215 (YGRKKRRLDPETGEFL), and SEQ ID NO: 216 (LDPETGEFLYGRKKRR).

Keap1 interacts with Nrf2 in a dimerized structure, wherein the DLG and ETGE regions of Nrf2 bind two identical Kelch domains. Without wishing to be bound by any particular theory, it is believed that a protein-like polymer having (i) a first peptide that inhibits binding to the DLG motif of Nrf2 and (i) a second peptide that inhibits binding to the ETGE motif of Nrf2, and/or a degree of polymerization of that allows for the protein-like polymer to bridge the gap between both Kelch domains of a Keap1 homodimer, significantly increases the binding interactions of the protein-like polymer, thereby increasing the inhibitory effect.

In some specific embodiments, the polymer comprises a tag for imaging and/or analysis. For example, each polymer segment S of formula (FX1a), (FX1b), (FX1c), (FX1d); (FX1e); (FX1f); or (FX1g) can independently comprise a tag for imaging and/or analysis. Similarly, each P² of formula (FX2a), (FX2b), or (FX2c) can independently comprise a tag for imaging and/or analysis. For example, the polymer can comprise a dye, radiolabeling, an imaging agent, tritiation, and the like.

After polymerization the inventive polymers may be characterized using any suitable technique(s). Typically, the inventive polymers are characterized by size-exclusion chromatography with multiangle light scattering (SEC-MALS), sometimes referred to as gel permeation chromatography (GPC), to ascertain degree of polymerization (DP) and molecular weight distribution (dispersity or Mw/Mn). Alternatively, or in addition to, the inventive polymers may be characterized by SDS-PAGE to ascertain degree of polymerization (DP) and molecular weight. Preferably, there is suitable agreement between the obtained DP and the theoretical DP based on the initial monomer-to-initiator ratio ([M]₀/[I]₀).

The inventive polymer can have any suitable degree of polymerization. If the degree of polymerization is too low, the polymer may not be resistant to enzymatic cleavage by proteases or may be cleared too rapidly from the body since the polymer's molecular weight would be lower than the clearance threshold through the kidney. Alternatively, if the degree of polymerization is too high, the peptide side chain groups displayed on the polymer may be too dense to engage their biological targets such as cell receptors, enzymes, etc. Typically, the polymer has a degree of polymerization of 2 to 1000 (e.g., 2 to 500, 2 to 250, 2 to 100, 2 to 50, 2 to 30, 5 to 1000, 5 to 500, 5 to 250, 5 to 100, 5 to 50, 5 to 30, 20 to 500, 20 to 250, 20 to 100, 20 to 50, or 20 to 30). In certain embodiments, the polymer has a degree of polymerization of 5 to 100. In preferred embodiments, the polymer has a degree of polymerization of 5 to 50. For example, the polymer can have a degree of polymerization of 5 or about 5, a degree of polymerization of 15 or about 15 (e.g., 17), a degree of polymerization of 30 or about 30, or a degree of polymerization of 50 or about 50. In certain embodiments, the polymer has a degree of polymerization of at least 20. Without wishing to be bound by any particular theory, it is believed that a degree of polymerization of at least 20 allows for the protein-like polymer to bridge the gap between both Kelch domains of a Keap1 homodimer, thereby increasing binding interactions by stably binding both Kelch domains simultaneously.

The inventive polymer can have any suitable weight average molecular weight. The polymers can have a weight average molecular weight of 2,000 kDa or less, for example, 1,800 kDa or less, 1,600 kDa or less, 1,400 kDa or less, 1,200 kDa or less, 1,000 kDa or less, 900 kDa, or less, 800 kDa, or less, 700 kDa or less, 600 kDa or less, 500 kDa or less, 250 kDa or less, 100 kDa or less, or 50 kDa or less. Alternatively, or in addition, the polymers can have a weight average molecular weight of 500 Da or more, for example, 1 kDa or more, 5 kDa or more, or 10 kDa or more. Thus, the polymers can have a weight average molecular weight bounded by any two of the aforementioned endpoints. For example, the polymers can have a weight average molecular weight of from 500 Da to 2,000 kDa, from 500 Da to 1,000 kDa, from 500 Da to 500 kDa, from 500 Da to 100 kDa, from 500 Da to 50 kDa, 1 kDa to 2,000 kDa, from 1 kDa to 1,000 kDa, from 1 kDa to 500 kDa, from 1 kDa to 100 kDa, from 1 kDa to 50 kDa, 5 kDa to 2,000 kDa, from 5 kDa to 1,000 kDa, from 5 kDa to 500 kDa, from 5 kDa to 100 kDa, from 5 kDa to 50 kDa, 10 kDa to 2,000 kDa, from 10 kDa to 1,000 kDa, from 10 kDa to 500 kDa, from 10 kDa to 100 kDa, or from 10 kDa to 50 kDa. In some embodiments, the inventive polymer has a weight average molecular weight of from 1 kDa to 50 kDa, 5 kDa to 50 kDa, or 10 kDa to 50 kDa.

Generally, the polymers described herein are characterized by a brush density of greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a brush density selected from the range 50% to 100%, optionally some embodiments a brush density selected from the range of 60% to 100%, optionally for some embodiments a brush density selected from the range of 70% to 100%, optionally some embodiments a brush density selected from the range of 80% to 100%, or optionally for some embodiments a brush density selected from the range of 90% to 100%.

The polymer can have any suitable peptide density. The polymer may be characterized by peptide density which refers to the percentage of the repeating units comprising a polymer backbone group covalently linked to at least one peptide. Thus, for each of the polymers characterized by the formula (FX2a), (FX2b), and (FX2c), the polymer density can be defined by the following formula:

$$\frac{{P}^{1}}{{P}^{1} + {P}^{2} + S} \times 100.$$

Generally, the polymers described herein are characterized by a peptide density of greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a peptide density selected from the range 50% to 100%, optionally some embodiments a peptide density selected from the range of 60% to 100%, optionally for some embodiments a peptide density selected from the range of 70% to 100%, optionally some embodiments a peptide density selected from the range of 80% to 100%, or optionally for some embodiments a peptide density selected from the range of 90% to 100%. In some embodiments, the brush density is equivalent to the peptide density.

In another aspect, the invention provides a pharmaceutical composition comprising one or more peptides and/or one or more polymers described herein. In some embodiments, the composition comprises one or more pharmaceutically acceptable excipients. For example, the peptides and/or polymers of the invention can be formulated for parenteral administration, such as intravenous (IV) administration or administration into a body cavity or lumen of an organ. Alternatively, the peptides and/or polymers can be injected intra-tumorally. Formulations for injection will commonly comprise a solution of the peptide and/or polymer dissolved in a pharmaceutically acceptable carrier. Among the acceptable vehicles and solvents that can be employed are water and an isotonic sodium chloride. In addition, sterile fixed oils can conventionally be employed as a solvent or suspending medium. For this purpose, any bland fixed oil can be employed including synthetic monoglycerides or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the peptide and/or polymer in these formulations can vary widely, and will be selected primarily based on fluid volumes, viscosities, body weight, and the like, in accordance with the particular mode of administration selected and the patient's needs. In certain embodiments, the concentration of a peptide and/or polymer in a solution formulation for injection will range from 0.1% (w/w) to 10% (w/w) or about 0.1% (w/w) to about 10% (w/w).

In some embodiments, the composition further comprises an additional Keap1 inhibitor or Nrf2 inducer. For example the composition can further comprise an additional small molecule drug such as dimethyl fumarate, tert-butylhydroquinone, DL-sulforaphane, or the like. Other small molecule Keap1 inhibitors or Nrf2 inducers will be readily apparent to those skill in the art. In certain embodiments, the composition further comprises an additional Keap1 inhibiting peptide. In other words, the composition can comprise a protein-like polymer described herein and an additional peptide.

In another aspect, the invention provides a method of treating or managing a condition comprising administering to a subject an effective amount of a peptide, polymer, and/or pharmaceutical composition described herein. The peptide, polymer, and/or pharmaceutical composition can be administered by oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. In some embodiments, the peptide, polymer, and/or pharmaceutical composition is administered intravenously, subcutaneously, intramuscularly, topically, orally, or a combination thereof.

The methods described herein can comprise contacting a target tissue of the subject with the peptide and/or polymer or a metabolite or product thereof, contacting a target cell of the subject with the peptide and/or polymer or a metabolite or product thereof, and/or contacting a target receptor of the subject with the peptide and/or polymer or a metabolite or product thereof. In some embodiments, the methods described herein can further comprise contacting a target receptor through two or more target receptor domains of the subject with the polymer or a metabolite or product thereof. In preferred embodiments, the peptides and/or polymers described herein pass through the cell membrane and contact an intracellular target. Without wishing to be bound by any particular theory, it is believe that the peptide/polymer structure and charge described herein play an integral role in providing cell permeability.

In some embodiments, the methods described herein interrupt the protein-protein interaction between Nuclear factor (erythroid-derived 2)-like 2 (Nrf2) and Kelch-like ECH-Associating protein 1 (Keap1) (e.g., by inhibiting binding to the DLG motif of Nrf2). Without wishing to be bound by any particular theory, inhibiting Keap1/Nrf2 binding can enhance the antioxidant and anti-inflammatory response to provide beneficial effects in both the central nervous system (CNS) and/or the non-central nervous system. Thus, the methods described herein can be used to treat and/or manage a condition associated with an inflammatory state, increased oxidative stress, autoimmune pathophysiology, chemo-preventative measures, neurodegeneration, or a combination thereof.

The method includes administering a therapeutically effective amount of a peptide, polymer, and/or composition described herein to a subject in need thereof. For example, the methods can include administering the peptide, polymer, and/or composition to provide a dose of from 10 ng/kg to 50 mg/kg to the subject. For example, the peptide and/or polymer dose can range from 5 mg/kg to 50 mg/kg, from 10 μg/kg to 5 mg/kg, or from 100 μg/kg to 1 mg/kg. The peptide and/or polymer dose can also lie outside of these ranges, depending on the particular peptide and/or polymer as well as the type of disease being treated. Frequency of administration can range from a single dose to multiple doses per week, or more frequently. In some embodiments, the peptide and/or polymer is administered from about once per month to about five times per week. In some embodiments, the peptide and/or polymer is administered once per week.

In some embodiments, the methods described herein can be used to treat or manage an autoimmune disease. For example, the methods described herein can be used to treat or manage multiple sclerosis, systemic lupus erythematous, Sjogren syndrome, rheumatoid arthritis, vitiligo, psoriasis, or the like.

In some embodiments, the methods described herein can be used to treat or manage a respiratory disease. For example, the methods described herein can be used to treat or manage COPD, emphysema, potential treatment for smokers, idiopathic pulmonary fibrosis, chronic sarcoidosis, hypersensitivity pneumonitis, or the like.

In some embodiments, the methods described herein can be used to treat or manage a gastrointestinal disease. For example, the methods described herein can be used to treat or manage ulcerative colitis, ulcers, prevent acetaminophen toxicity, non-alcoholic steatohepatitis, primary biliary cholangitis, cirrhosis, type 2 diabetes, diabetic nephropathy, or the like.

In some embodiments, the methods described herein can be used to treat or manage a cardiovascular disease. For example, the methods described herein can be used to treat or manage cardiac ischemia-reperfusion injury, heart failure, atherosclerosis, or the like.

In some embodiments, the methods described herein can be used to treat or manage a neurodegenerative disease. For example, the methods described herein can be used to treat or manage Alzheimer's disease, Parkinson's disease, amyotrophic lateral sclerosis (ALS), Huntington's disease, Friedreich ataxia, frontotemporal lobar degeneration, or the like.

The invention may be further set forth and understood in view of the following non-limiting examples and embodiments which one having skill in the art will readily understand are intended to illustrate specific aspects of the invention.

### Example 1

This example provides the amino acid residue interaction analysis for the binding complex between the Kelch domain of the Keap1 protein and SEQ ID NO: 218 (MDLIDILWRQDIDLGVSREVFDFS).

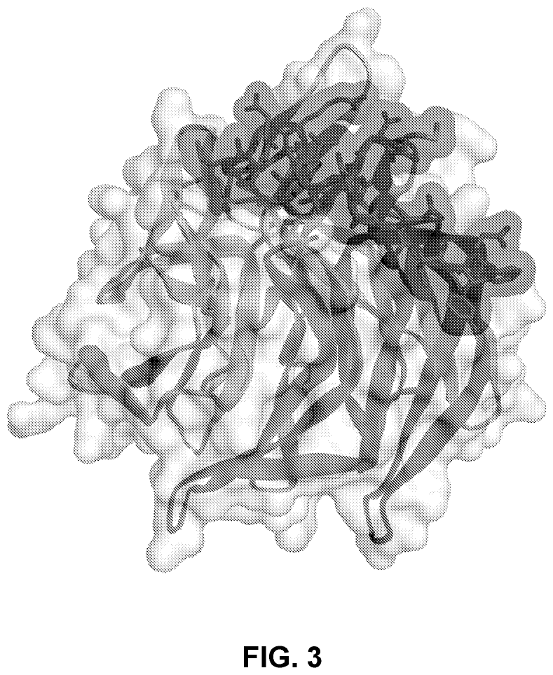

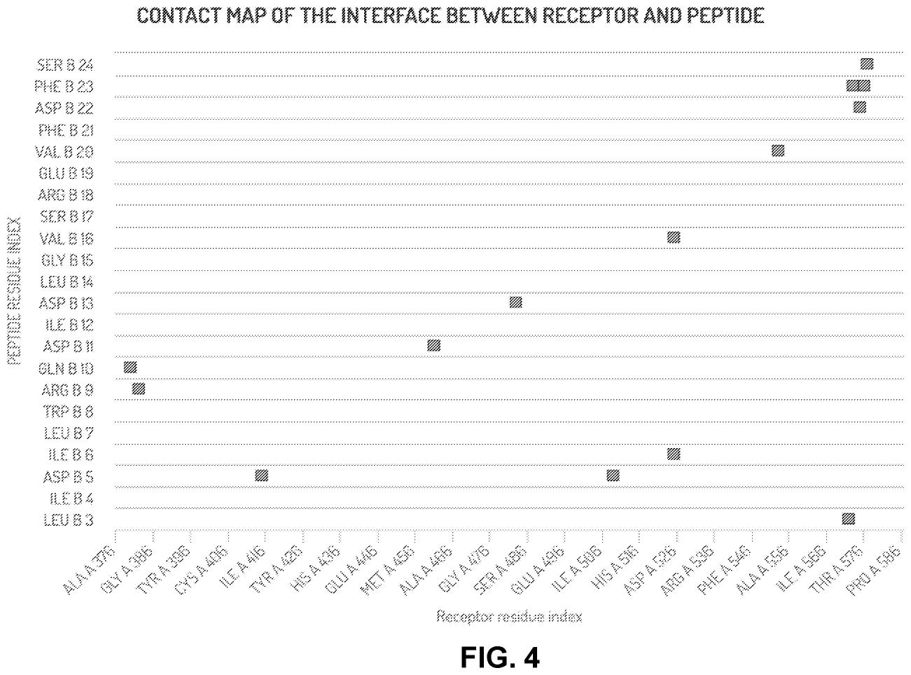

Peptide sequence SEQ ID NO: 218 (MDLIDILWRQDIDLGVSREVFDFS) was evaluated for binding interactions with Keap1 representative protein sequence SEQ ID NO: 217 (RLIYTAGGYFRQSLSYLEAYNPSDGTWLRRLADLQVPRSGLAGCVVGGLLYAVGGRN NSPDGNTDSSALDCYNPMTNQWSPCAPMSVPRNRIGVGVIDGHIYAVGGSHGCIHHNSV ERYEPERDEWHLVAPMLTRRIGVGVAVLNRLLYAVGGFDGTNRLNSAECYYPERNEWR MITAMNTIRSGAGVCVLHNCIYAAGGYDGQDQLNSVERYDVATATWTFVAPMKHRRS ALGITVHQGRIYVLGGYDGHTFLDSVECYDPDTDTWSEVTRMTSGRSGVGVAVT) using in-silico simulations (50 cycles) with CABS-Dock software, which is capable of simultaneous prediction of binding sites and protein-peptide docking through coarse-gain methods, and the resulting complex is set forth in FIG. 3. Using the predicted complex set forth in FIG. 3, a contact map of the amino acid interactions of the interface between the Kelch domain of the Keap1 protein and SEQ ID NO: 218 (MDLIDILWRQDIDLGVSREVFDFS) was generated and the results are set forth in FIG. 4.

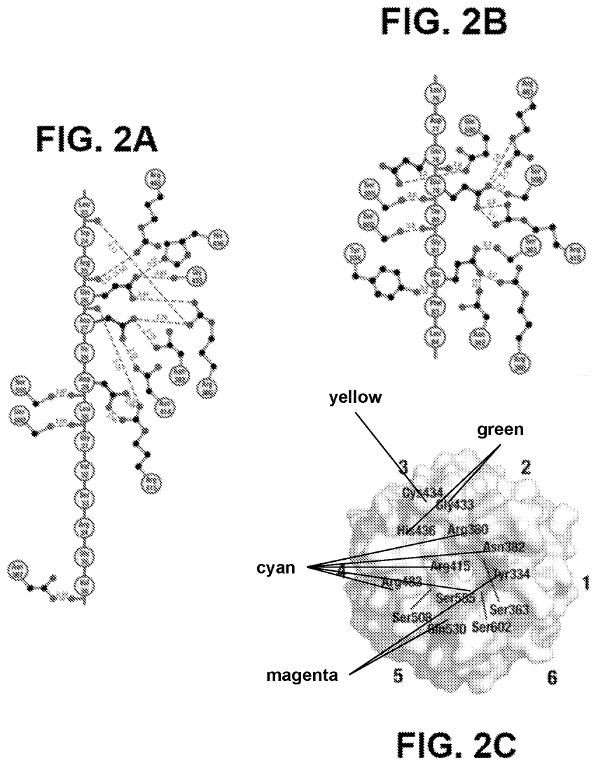

As is apparent from the results set forth in FIG. 4, SEQ ID NO: 218 (MDLIDILWRQDIDLGVSREVFDFS) has 14 total interactions with a distance of less than 3 Å with the Kelch domain of the Keap1 protein. Of the seven documented amino acid residues (i.e., His436, Gly433, Arg483, Arg415, Ser555, Arg380, and Asn382) of the Keap1 protein known to bind to the DLG motif with a distance of less than 3 Å, as disclosed by Fukutomi et al. (Mol. Cell. Biol., 34(5): 832-846 (2014)) and shown in FIGS. 2A-2C, SEQ ID NO: 218 (MDLIDILWRQDIDLGVSREVFDFS) has binding interactions (<3 Å) with four of these residues (i.e., Arg483, Arg415, Arg380, and Asn382). Without wishing to be bound by any particular theory, it is believed that interactions with the arginine residues in the Kelch domain binding pocket are the most important. Furthermore, the simulation results provided in FIG. 4 show the most notable interactions of SEQ ID NO: 218 (MDLIDILWRQDIDLGVSREVFDFS) with the Kelch domain. Notably, FIG. 4. shows that the key residues from the Nrf2 sequence include but are not limited to: Leu19, Asp21, Ile22, Arg25, Gln26, Asp27, Asp29, Val32, Val36, Asp38, Phe39, Ser40.

### Example 2

This example provides the amino acid residue interaction analysis for the binding complex between the Kelch domain of the Keap1 protein and SEQ ID NO: 2 (RQDIDLGVSRR).

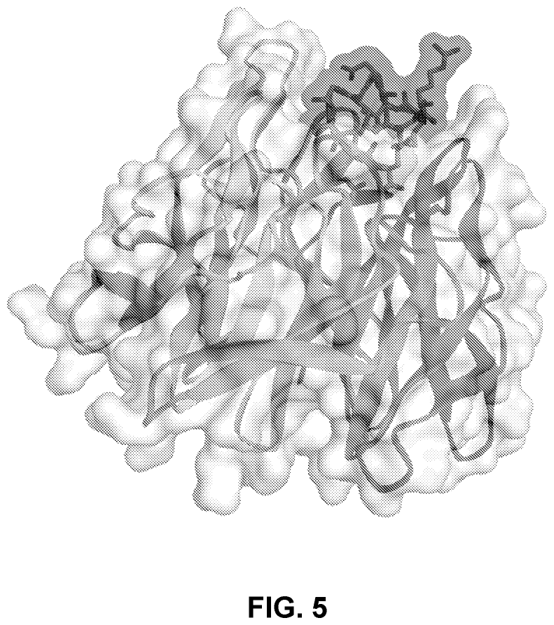

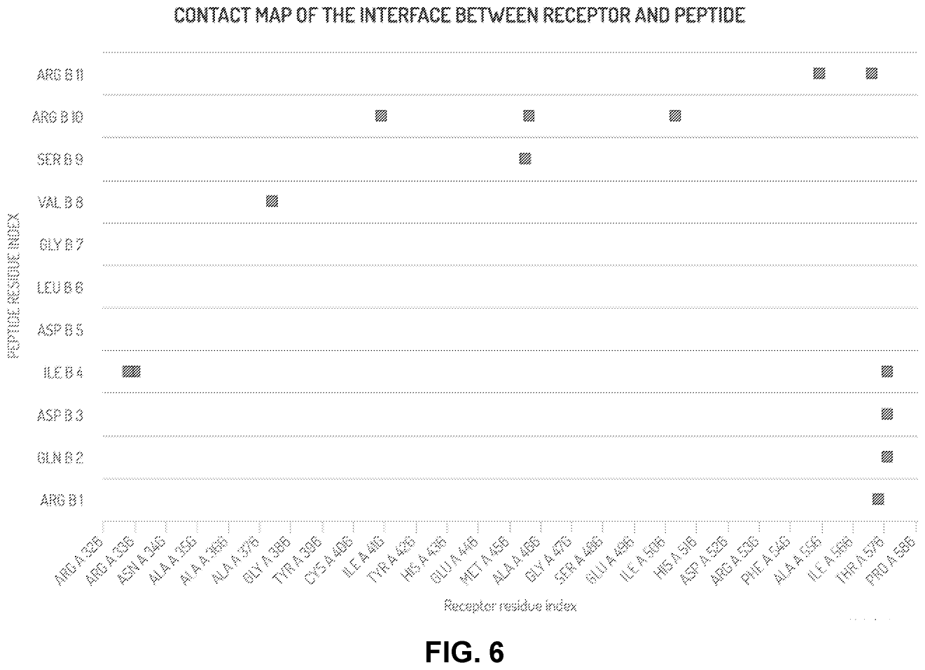

Peptide sequence SEQ ID NO: 2 (RQDIDLGVSRR) was evaluated for binding interactions with Keap1 representative protein sequence SEQ ID NO: 217 (RLIYTAGGYFRQSLSYLEAYNPSDGTWLRRLADLQVPRSGLAGCVVGGLLYAVGGRN NSPDGNTDSSALDCYNPMTNQWSPCAPMSVPRNRIGVGVIDGHIYAVGGSHGCIHHNSV ERYEPERDEWHLVAPMLTRRIGVGVAVLNRLLYAVGGFDGTNRLNSAECYYPERNEWR MITAMNTIRSGAGVCVLHNCIYAAGGYDGQDQLNSVERYDVATATWTFVAPMKHRRS ALGITVHQGRIYVLGGYDGHTFLDSVECYDPDTDTWSEVTRMTSGRSGVGVAVT) using in-silico simulations (50 cycles) with CABS-Dock software, which is capable of simultaneous prediction of binding sites and protein-peptide docking through coarse-gain methods, and the resulting complex is set forth in FIG. 5. Using the predicted complex set forth in FIG. 5, a contact map of the amino acid interactions of the interface between the Kelch domain of the Keap1 protein and SEQ ID NO: 2 (RQDIDLGVSRR) was generated and the results are set forth in FIG. 6.

As is apparent from the results set forth in FIG. 6, SEQ ID NO: 2 (RQDIDLGVSRR) has 13 total interactions with a distance of less than 3 Å with the Kelch domain of the Keap1 protein. Of the seven documented amino acid residues (i.e., His436, Gly433, Arg483, Arg415, Ser555, Arg380, and Asn382) of the Keap1 protein known to bind to the DLG motif with a distance of less than 3 Å, as disclosed by Fukutomi et al. (Mol. Cell. Biol., 34(5): 832-846 (2014)) and shown in FIGS. 2A-2C, SEQ ID NO: 2 (RQDIDLGVSRR) has binding interactions (<3 Å) with three of these residues (i.e., Arg 415, Ser555, and Arg380). Without wishing to be bound by any particular theory, it is believed that interactions with the arginine residues in the Kelch domain binding pocket are the most important.

### Example 3

This example provides the amino acid residue interaction analysis for the binding complex between the Kelch domain of the Keap1 protein and SEQ ID NO: 1 (RQDIDLGVSR).

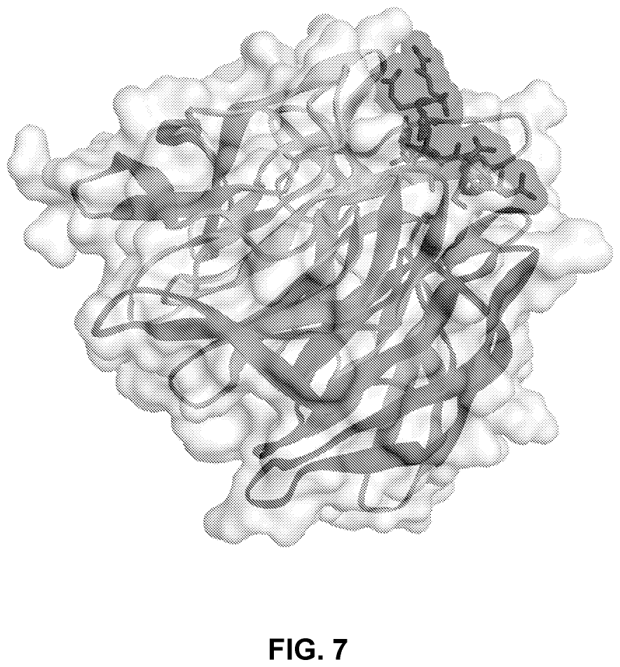

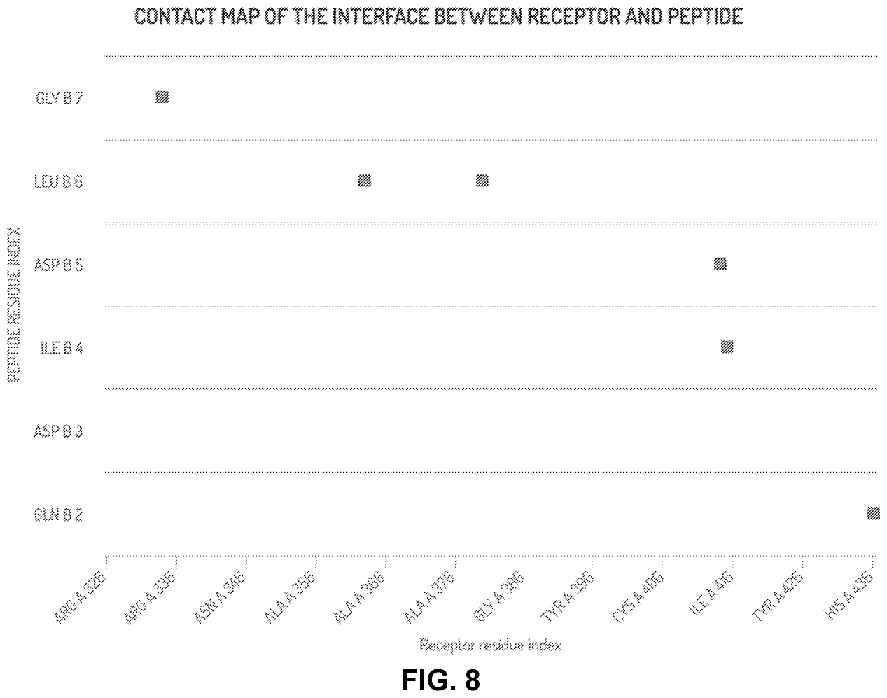

Peptide sequence SEQ ID NO: 1 (RQDIDLGVSR) was evaluated for binding interactions with Keap1 representative protein sequence SEQ ID NO: 217 (RLIYTAGGYFRQSLSYLEAYNPSDGTWLRRLADLQVPRSGLAGCVVGGLLYAVGGRN NSPDGNTDSSALDCYNPMTNQWSPCAPMSVPRNRIGVGVIDGHIYAVGGSHGCIHHNSV ERYEPERDEWHLVAPMLTRRIGVGVAVLNRLLYAVGGFDGTNRLNSAECYYPERNEWR MITAMNTIRSGAGVCVLHNCIYAAGGYDGQDQLNSVERYDVATATWTFVAPMKHRRS ALGITVHQGRIYVLGGYDGHTFLDSVECYDPDTDTWSEVTRMTSGRSGVGVAVT) using in-silico simulations (50 cycles) with CABS-Dock software, which is capable of simultaneous prediction of binding sites and protein-peptide docking through coarse-gain methods, and the resulting complex is set forth in FIG. 7. Using the predicted complex set forth in FIG. 7, a contact map of the amino acid interactions of the interface between the Kelch domain of the Keap1 protein and SEQ ID NO: 1 (RQDIDLGVSR) was generated and the results are set forth in FIG. 8.

As is apparent from the results set forth in FIG. 8, SEQ ID NO: 1 (RQDIDLGVSR) has 6 total interactions with a distance of less than 3 Å with the Kelch domain of the Keap1 protein. Of the seven documented amino acid residues (i.e., His436, Gly433, Arg483, Arg415, Ser555, Arg380, and Asn382) of the Keap1 protein known to bind to the DLG motif with a distance of less than 3 Å, as disclosed by Fukutomi et al. (Mol. Cell. Biol., 34(5): 832-846 (2014)) and shown in FIGS. 2A-2C, SEQ ID NO: 1 (RQDIDLGVSR) has binding interactions (<3 Å) with three of these residues (i.e., His436, Arg480 and Arg415). Without wishing to be bound by any particular theory, it is believed that interactions with the arginine residues in the Kelch domain binding pocket are the most important.

### Example 4

This example provides the amino acid residue interaction analysis for the binding complex between the Kelch domain of the Keap1 protein and SEQ ID NO: 219 (ILWRQDIDLGVSRR).

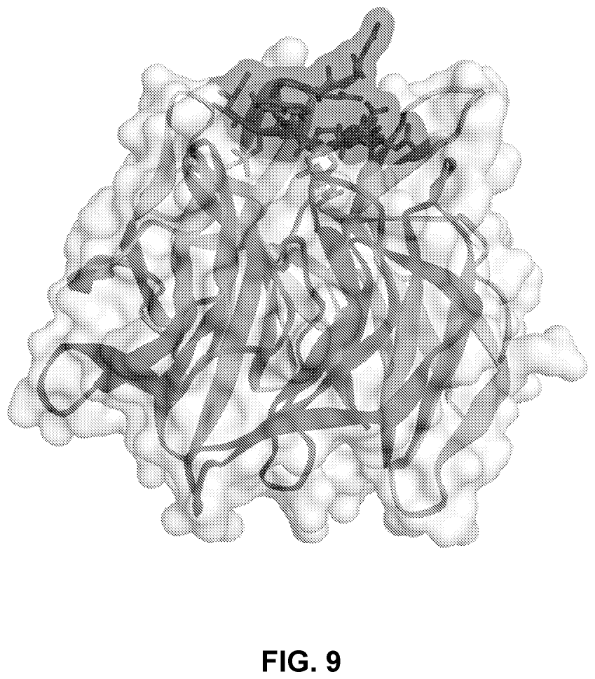

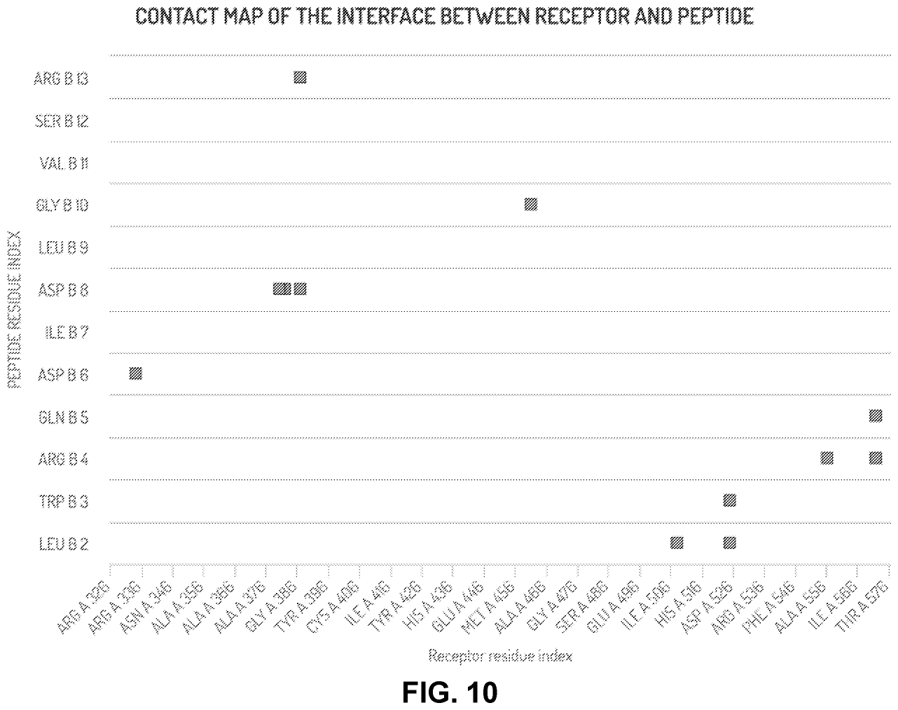

Peptide sequence SEQ ID NO: 219 (ILWRQDIDLGVSRR) was evaluated for binding interactions with Keap1 representative protein sequence SEQ ID NO: 217 (RLIYTAGGYFRQSLSYLEAYNPSDGTWLRRLADLQVPRSGLAGCVVGGLLYAVGGRN NSPDGNTDSSALDCYNPMTNQWSPCAPMSVPRNRIGVGVIDGHIYAVGGSHGCIHHNSV ERYEPERDEWHLVAPMLTRRIGVGVAVLNRLLYAVGGFDGTNRLNSAECYYPERNEWR MITAMNTIRSGAGVCVLHNCIYAAGGYDGQDQLNSVERYDVATATWTFVAPMKHRRS ALGITVHQGRIYVLGGYDGHTFLDSVECYDPDTDTWSEVTRMTSGRSGVGVAVT) using in-silico simulations (50 cycles) with CABS-Dock software, which is capable of simultaneous prediction of binding sites and protein-peptide docking through coarse-gain methods, and the resulting complex is set forth in FIG. 9. Using the predicted complex set forth in FIG. 9, a contact map of the amino acid interactions of the interface between the Kelch domain of the Keap1 protein and SEQ ID NO: 219 (ILWRQDIDLGVSRR) was generated and the results are set forth in FIG. 10.

As is apparent from the results set forth in FIG. 10, SEQ ID NO: 219 (ILWRQDIDLGVSRR) has 12 total interactions with a distance of less than 3 Å with the Kelch domain of the Keap1 protein. Of the seven documented amino acid residues (i.e., His436, Gly433, Arg483, Arg415, Ser555, Arg380, and Asn382) of the Keap1 protein known to bind to the DLG motif with a distance of less than 3 Å, as disclosed by Fukutomi et al. (Mol. Cell. Biol., 34(5): 832-846 (2014)) and shown in FIGS. 2A-2C, SEQ ID NO: 219 (ILWRQDIDLGVSRR) has binding interactions (<3 Å) with two of these residues (i.e., Arg380 and Asn382). Without wishing to be bound by any particular theory, it is believed that interactions with the arginine residues in the Kelch domain binding pocket are the most important.

### Example 5

This example provides the amino acid residue interaction analysis for the binding complex between the Kelch domain of the Keap1 protein and SEQ ID NO: 220 (ILWRQDIDLGVSR).

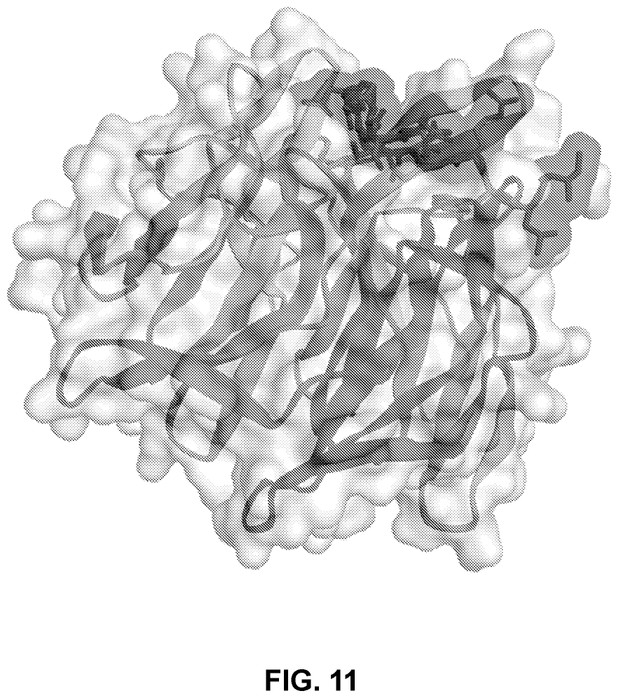

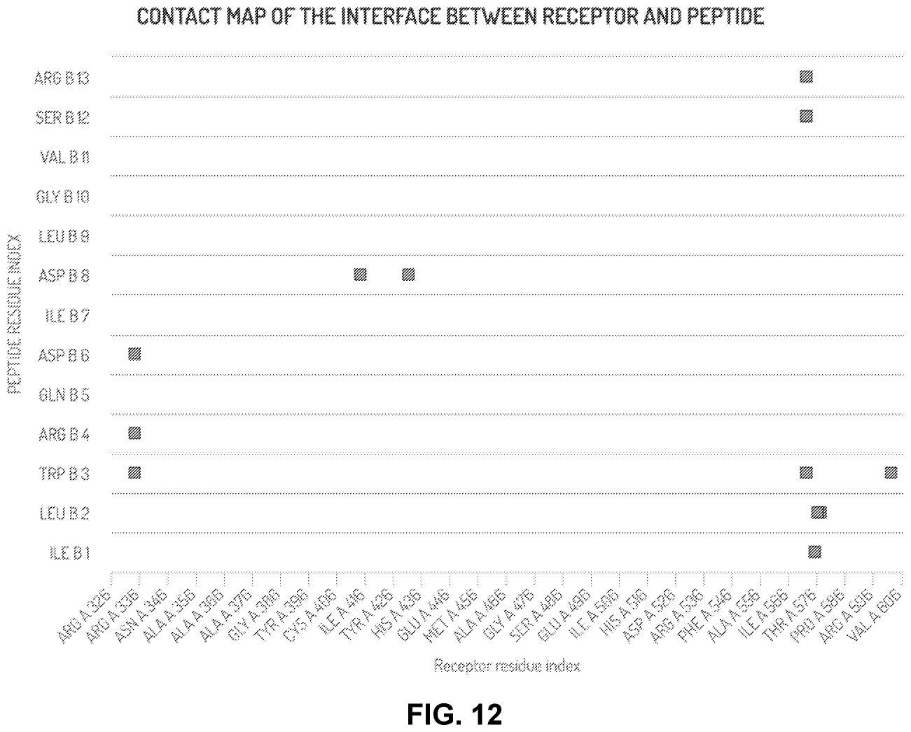

Peptide sequence SEQ ID NO: 220 (ILWRQDIDLGVSR) was evaluated for binding interactions with Keap1 representative protein sequence SEQ ID NO: 217 (RLIYTAGGYFRQSLSYLEAYNPSDGTWLRRLADLQVPRSGLAGCVVGGLLYAVGGRN NSPDGNTDSSALDCYNPMTNQWSPCAPMSVPRNRIGVGVIDGHIYAVGGSHGCIHHNSV ERYEPERDEWHLVAPMLTRRIGVGVAVLNRLLYAVGGFDGTNRLNSAECYYPERNEWR MITAMNTIRSGAGVCVLHNCIYAAGGYDGQDQLNSVERYDVATATWTFVAPMKHRRS ALGITVHQGRIYVLGGYDGHTFLDSVECYDPDTDTWSEVTRMTSGRSGVGVAVT) using in-silico simulations (50 cycles) with CABS-Dock software, which is capable of simultaneous prediction of binding sites and protein-peptide docking through coarse-gain methods, and the resulting complex is set forth in FIG. 11. Using the predicted complex set forth in FIG. 11, a contact map of the amino acid interactions of the interface between the Kelch domain of the Keap1 protein and SEQ ID NO: 220 (ILWRQDIDLGVSR) was generated and the results are set forth in FIG. 12.

As is apparent from the results set forth in FIG. 12, SEQ ID NO: 220 (ILWRQDIDLGVSR) has 12 total interactions with a distance of less than 3 Å with the Kelch domain of the Keap1 protein. Of the seven documented amino acid residues (i.e., His436, Gly433, Arg483, Arg415, Ser555, Arg380, and Asn382) of the Keap1 protein known to bind to the DLG motif with a distance of less than 3 Å, as disclosed by Fukutomi et al. (Mol. Cell. Biol., 34(5): 832-846 (2014)) and shown in FIGS. 2A-2C, SEQ ID NO: 220 (ILWRQDIDLGVSR) has binding interactions (<3 Å) with none of these residues. Without wishing to be bound by any particular theory, it is believed that interactions with the arginine residues in the Kelch domain binding pocket are the most important.

### Example 6

This example provides the amino acid residue interaction analysis for the binding complex between the Kelch domain of the Keap1 protein and SEQ ID NO: 221 (LWRQDIDLGVSRR).

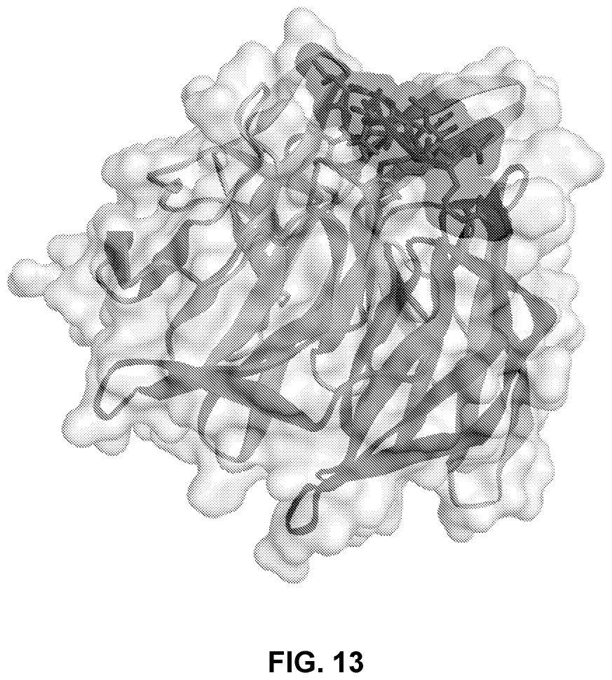

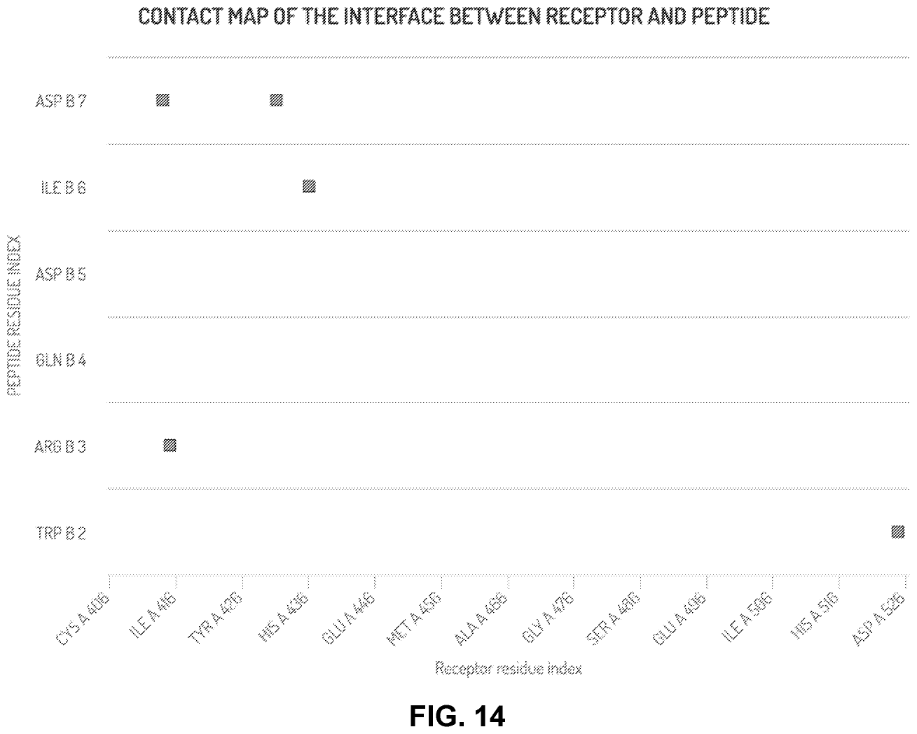

Peptide sequence SEQ ID NO: 221 (LWRQDIDLGVSRR) was evaluated for binding interactions with Keap1 representative protein sequence SEQ ID NO: 217 (RLIYTAGGYFRQSLSYLEAYNPSDGTWLRRLADLQVPRSGLAGCVVGGLLYAVGGRN NSPDGNTDSSALDCYNPMTNQWSPCAPMSVPRNRIGVGVIDGHIYAVGGSHGCIHHNSV ERYEPERDEWHLVAPMLTRRIGVGVAVLNRLLYAVGGFDGTNRLNSAECYYPERNEWR MITAMNTIRSGAGVCVLHNCIYAAGGYDGQDQLNSVERYDVATATWTFVAPMKHRRS ALGITVHQGRIYVLGGYDGHTFLDSVECYDPDTDTWSEVTRMTSGRSGVGVAVT) using in-silico simulations (50 cycles) with CABS-Dock software, which is capable of simultaneous prediction of binding sites and protein-peptide docking through coarse-gain methods, and the resulting complex is set forth in FIG. 13. Using the predicted complex set forth in FIG. 13, a contact map of the amino acid interactions of the interface between the Kelch domain of the Keap1 protein and SEQ ID NO: 221 (LWRQDIDLGVSRR) was generated and the results are set forth in FIG. 14.

As is apparent from the results set forth in FIG. 14, SEQ ID NO: 221 (LWRQDIDLGVSRR) has 5 total interactions with a distance of less than 3 Å with the Kelch domain of the Keap1 protein. Of the seven documented amino acid residues (i.e., His436, Gly433, Arg483, Arg415, Ser555, Arg380, and Asn382) of the Keap1 protein known to bind to the DLG motif with a distance of less than 3 Å, as disclosed by Fukutomi et al. (Mol. Cell. Biol., 34(5): 832-846 (2014)) and shown in FIGS. 2A-2C, SEQ ID NO: 221 (LWRQDIDLGVSRR) has binding interactions (<3 Å) with two of these residues (i.e., His436 and Arg415). Without wishing to be bound by any particular theory, it is believed that interactions with the arginine residues in the Kelch domain binding pocket are the most important.

### Example 7

This example provides the amino acid residue interaction analysis for the binding complex between the Kelch domain of the Keap1 protein and SEQ ID NO: 222 (LWRQDIDLGVSR).

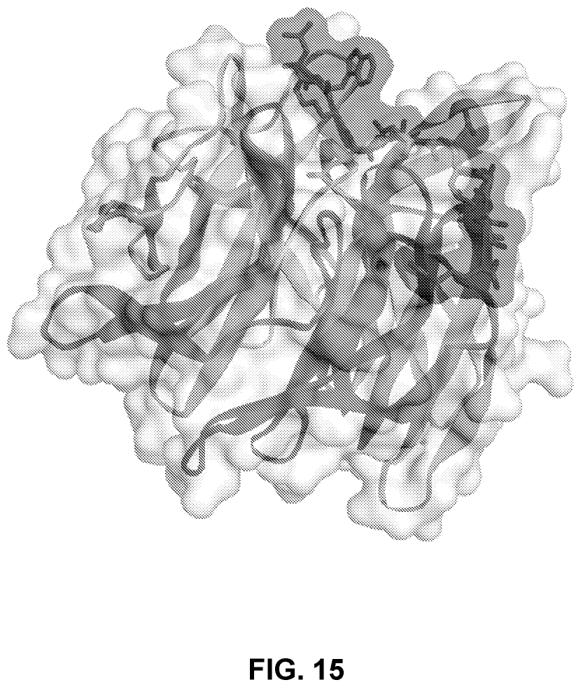

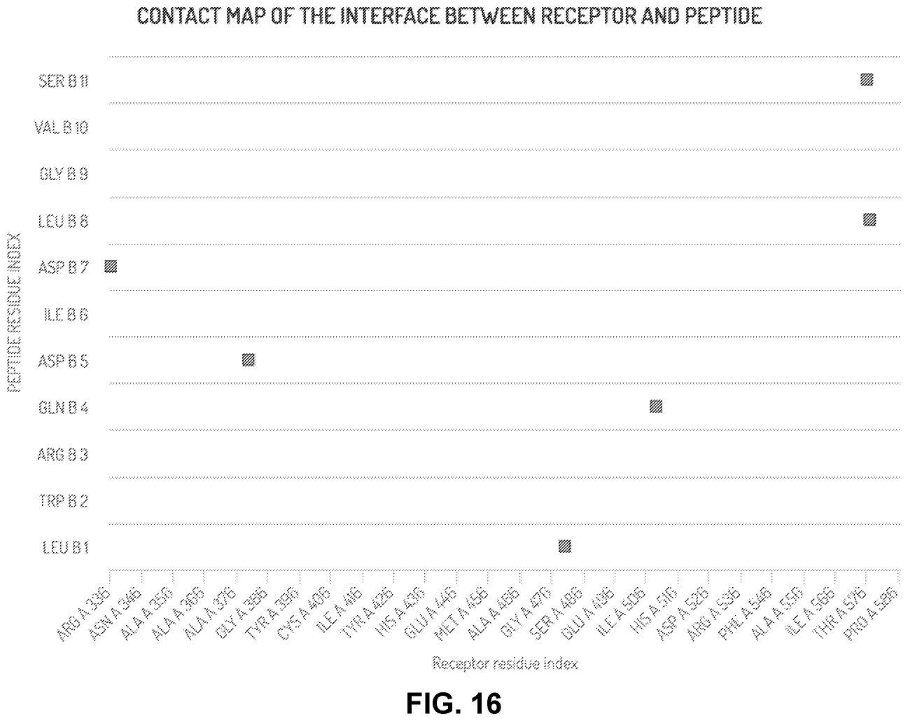

Peptide sequence SEQ ID NO: 222 (LWRQDIDLGVSR) was evaluated for binding interactions with Keap1 representative protein sequence SEQ ID NO: 217 (RLIYTAGGYFRQSLSYLEAYNPSDGTWLRRLADLQVPRSGLAGCVVGGLLYAVGGRN NSPDGNTDSSALDCYNPMTNQWSPCAPMSVPRNRIGVGVIDGHIYAVGGSHGCIHHNSV ERYEPERDEWHLVAPMLTRRIGVGVAVLNRLLYAVGGFDGTNRLNSAECYYPERNEWR MITAMNTIRSGAGVCVLHNCIYAAGGYDGQDQLNSVERYDVATATWTFVAPMKHRRS ALGITVHQGRIYVLGGYDGHTFLDSVECYDPDTDTWSEVTRMTSGRSGVGVAVT) using in-silico simulations (50 cycles) with CABS-Dock software, which is capable of simultaneous prediction of binding sites and protein-peptide docking through coarse-gain methods, and the resulting complex is set forth in FIG. 15. Using the predicted complex set forth in FIG. 15, a contact map of the amino acid interactions of the interface between the Kelch domain of the Keap1 protein and SEQ ID NO: 222 (LWRQDIDLGVSR) was generated and the results are set forth in FIG. 16.

As is apparent from the results set forth in FIG. 16, SEQ ID NO: 222 (LWRQDIDLGVSR) has 6 total interactions with a distance of less than 3 Å with the Kelch domain of the Keap1 protein. Of the seven documented amino acid residues (i.e., His436, Gly433, Arg483, Arg415, Ser555, Arg380, and Asn382) of the Keap1 protein known to bind to the DLG motif with a distance of less than 3 Å, as disclosed by Fukutomi et al. (Mol. Cell. Biol., 34(5): 832-846 (2014)) and shown in FIGS. 2A-2C, SEQ ID NO: 222 (LWRQDIDLGVSR) has binding interactions (<3 Å) with one of these residues (i.e., Arg380).

### Example 8

This example summarizes the results from the amino acid residue interaction analyses performed in Examples 1-7. More particularly, the peptide sequence charge, peptide total interactions (<3 Å), and number of interactions (<3 Å) with documented amino acid residues (i.e., His436, Gly433, Arg483, Arg415, Ser555, Arg380, and Asn382) of the Keap1 protein known to bind to the DLG motif with a distance of less than 3 Å are set forth in Table 1.

TABLE 1
Summary of Amino Acid Residue Interactions
Between DLG Peptides and Kelch Domain

| | | | Total | Documented |
|---|---|---|---|---|
| | | Peptide | Interactions | Interactions |
| Example | Peptide Sequence | Charge | (<3 Å) | (<3 Å) |
| 1 | SEQ ID NO: 218 | −4 | 14 | 4 |
| 2 | SEQ ID NO: 2 | +1 | 13 | 3 |
| 3 | SEQ ID NO: 1 | 0 | 6 | 3 |
| 4 | SEQ ID NO: 219 | +1 | 12 | 2 |
| 5 | SEQ ID NO: 220 | 0 | 12 | 0 |
| 6 | SEQ ID NO: 221 | +1 | 5 | 2 |
| 7 | SEQ ID NO: 222 | 0 | 6 | 1 |

As is apparent from the results set for the in Table 1, SEQ ID NO: 218 (MDLIDILWRQDIDLGVSREVFDFS) and SEQ ID NO: 2 (RQDIDLGVSRR) had the highest total binding interactions with the Kelch domain of the Keap1 protein. In addition, SEQ ID NO: 2 (RQDIDLGVSRR) also has a net cationic charge, indicating that SEQ ID NO: 2 (RQDIDLGVSRR) may provide the most suitable combination of binding affinity, cell penetrating potential, and solubility amongst the peptides tested.

### Example 9

This example shows the binding specificity between the Kelch domain and the following seven peptides: SEQ ID NO: 220 (ILWRQDIDLGVSR), SEQ ID NO: 219 (ILWRQDIDLGVSRR), SEQ ID NO: 222 (LWRQDIDLGVSR), SEQ ID NO: 221 (LWRQDIDLGVSRR), SEQ ID NO: 1 (RQDIDLGVSR), SEQ ID NO: 2 (RQDIDLGVSRR), and SEQ ID NO: 218 (MDLIDILWRQDIDLGVSREVFDFS).

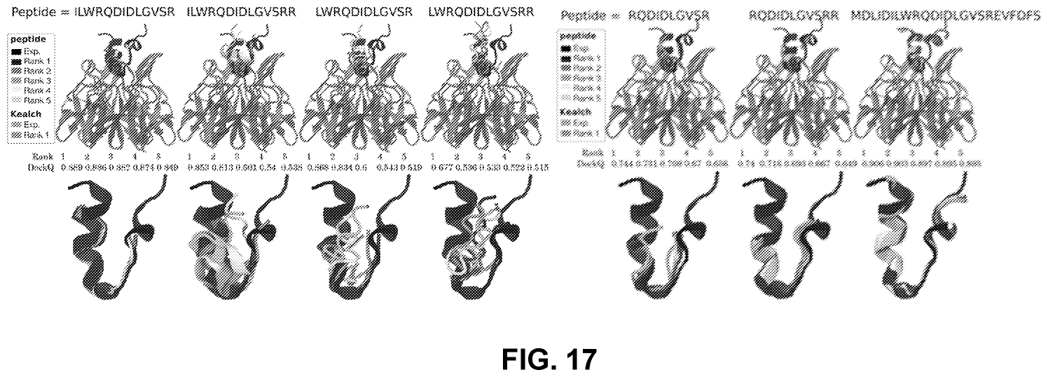

Binding specificity calculations and modeling were performed using Alphafold-multimer 2.1.1 for peptides SEQ ID NO: 220 (ILWRQDIDLGVSR), SEQ ID NO: 219 (ILWRQDIDLGVSRR), SEQ ID NO: 222 (LWRQDIDLGVSR), SEQ ID NO: 221 (LWRQDIDLGVSRR), SEQ ID NO: 1 (RQDIDLGVSR), SEQ ID NO: 2 (RQDIDLGVSRR), and SEQ ID NO: 218 (MDLIDILWRQDIDLGVSREVFDFS), and the results are set forth in FIG. 17.

As shown in the top row in FIG. 17, Alphafold-multimer provides the top five predicted structures (Ranks 1-5) for the peptides when complexed with the experimental structure of SEQ ID NO: 218 (MDLIDILWRQDIDLGVSREVFDFS), which are aligned using the backbone atoms of the Kelch domain only. Also included are the model confidence scores (1≥DockQ≥0) for the five predicted structures where a higher score (e.g., Rank 1) stands for a higher confidence. As evidenced by FIG. 17, the five predicted structures are well preserved for peptides SEQ ID NO: 220 (ILWRQDIDLGVSR), SEQ ID NO: 1 (RQDIDLGVSR), SEQ ID NO: 2 (RQDIDLGVSRR), and SEQ ID NO: 218 (MDLIDILWRQDIDLGVSREVFDFS), suggesting their specific binding feature with the Kelch domain. However, the five predicted structures for peptides SEQ ID NO: 219 (ILWRQDIDLGVSRR), SEQ ID NO: 222 (LWRQDIDLGVSR), and SEQ ID NO: 221 (LWRQDIDLGVSRR) are random with low DockQ scores indicating that the binding of SEQ ID NO: 219 (ILWRQDIDLGVSRR), SEQ ID NO: 222 (LWRQDIDLGVSR), and SEQ ID NO: 221 (LWRQDIDLGVSRR) with the Kelch domain is less stable.

The bottom row of FIG. 17 shows the five predicted structures (backbone only) for each peptide, superimposed with the experimental structure of SEQ ID NO: 218 (MDLIDILWRQDIDLGVSREVFDFS), which binds the Kelch domain.

These results show that the Kelch-peptide binding structures are highly preserved for SEQ ID NO: 220 (ILWRQDIDLGVSR), SEQ ID NO: 1 (RQDIDLGVSR), and SEQ ID NO: 2 (RQDIDLGVSRR), evidencing the specific binding feature of these three Kelch-peptide complexes. When considering the peptide interactions with Keap1 along with cell penetrating potential these simulations also validate the conclusions shown in Example 8 wherein SEQ ID NO: 2 (RQDIDLGVSRR), may provide the most suitable combination of binding affinity, cell penetrating potential, and solubility amongst the peptides tested.

### Example 10

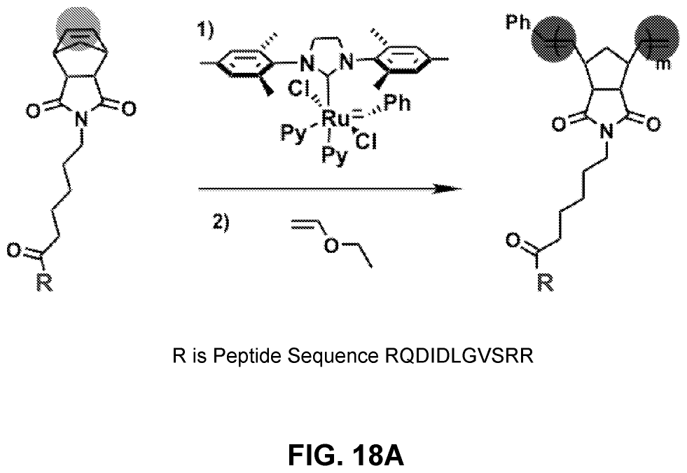

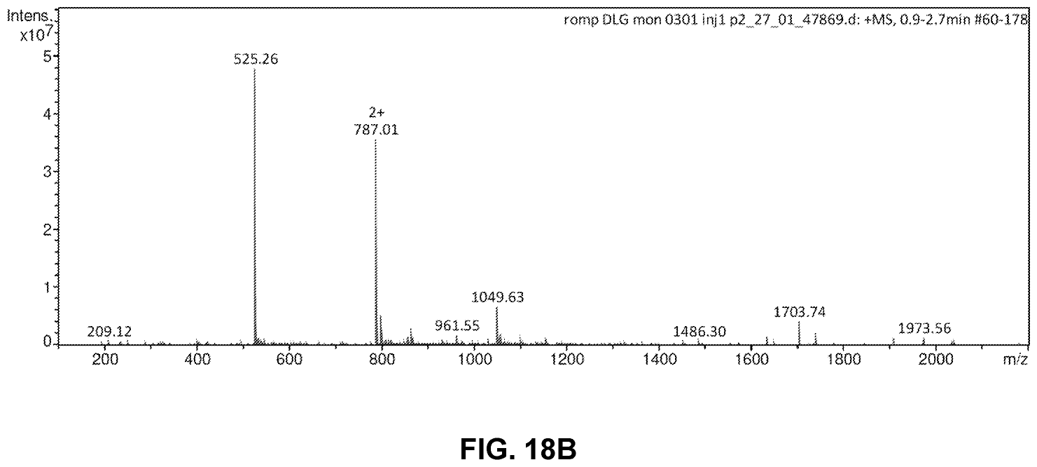

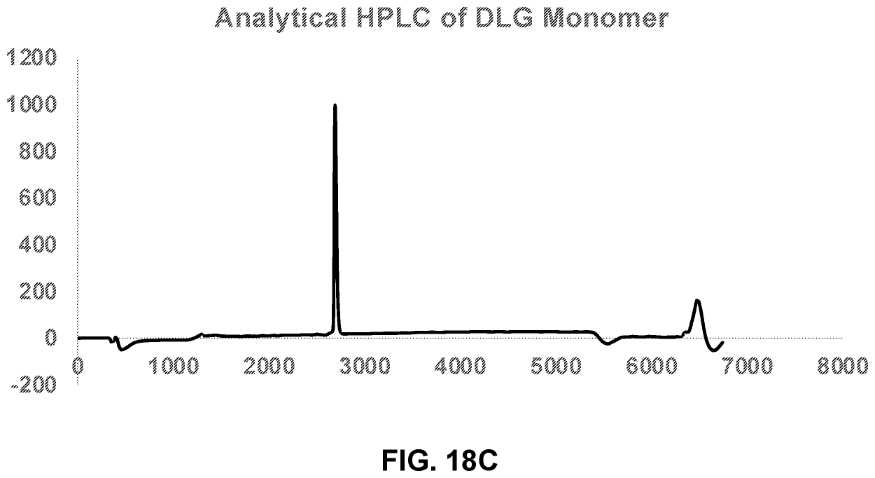

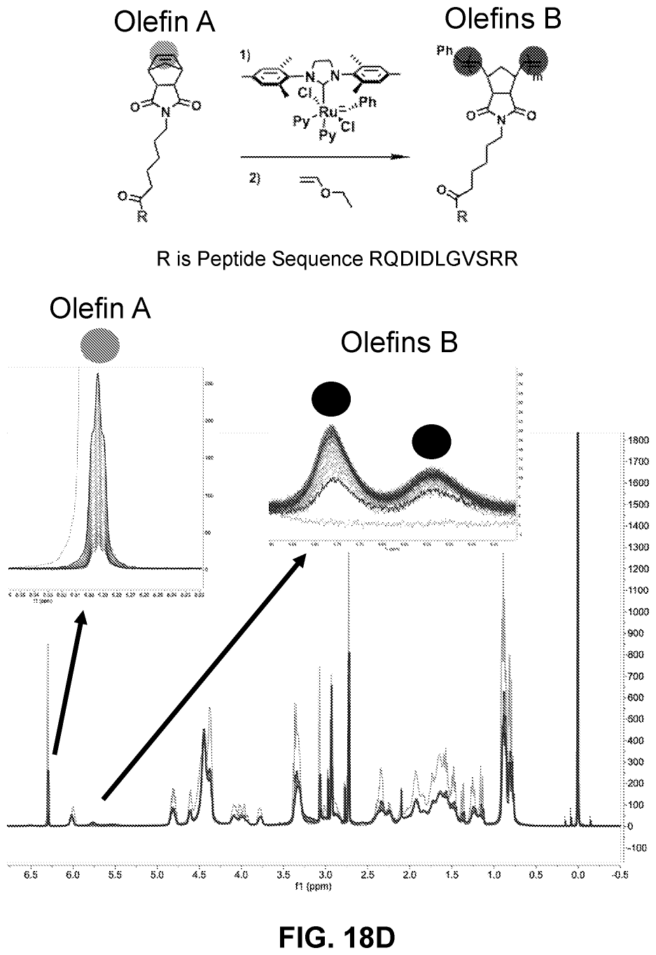

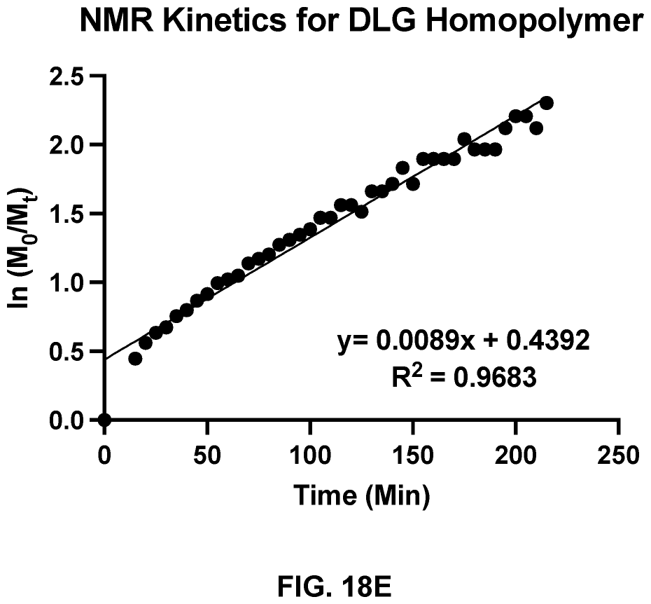

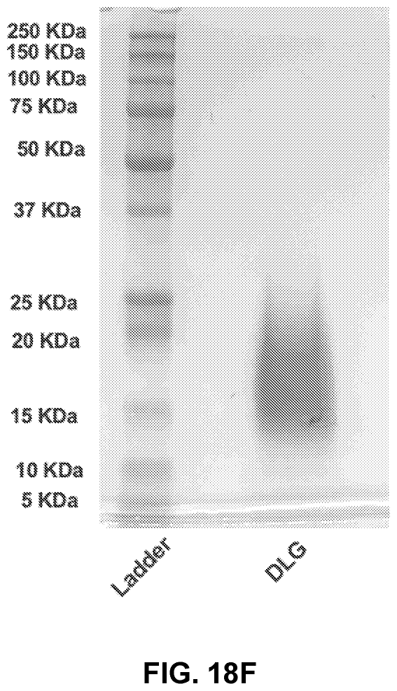

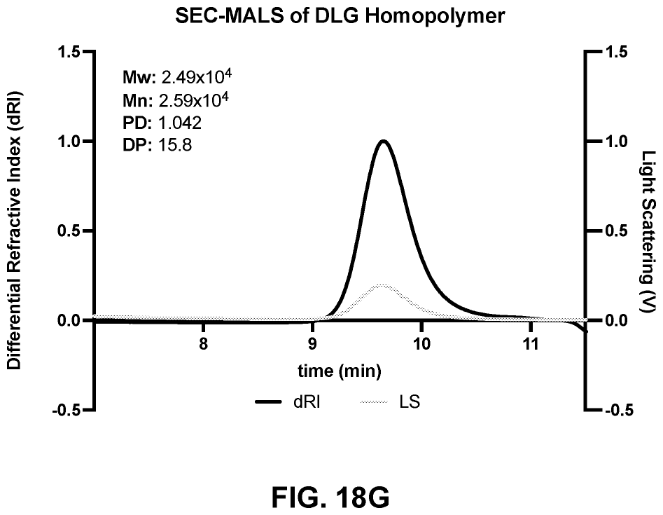

This example provides an exemplary synthesis of a polynorbornene dicarboxyimide-based brush polymer comprising the peptide SEQ ID NO: 2 (RQDIDLGVSRR), as depicted in FIG. 18A.

Peptide SEQ ID NO: 2 (RQDIDLGVSRR) was synthesized using standard solid phase peptide synthesis (SPPS) procedures on an AAPPTec Focus XC automated synthesizer. The peptide was prepared on Rink amide MBHA resin with a typical SPPS procedure involving FMOC deprotection with 20% methylpiperidine in DMF (one 5 min deprotection followed by one 15 min deprotection), and 45 min amide couplings using 3.75 eq. of the FMOC-protected, and side chain-protected amino acid, 4 eq. of HBTU and 8 eq. of DIPEA.

The peptide monomer depicted in FIG. 18A was prepared by amide coupling to N-(hexanoic acid)-cis-5-norbornene-exo-dicarboximide at the N-terminus of peptide SEQ ID NO: 2 (RQDIDLGVSRR). Following completion of the synthesis, the peptide was cleaved from the resin by treatment with TFA/H₂O/TIPS in a 9.5:2.5:2.5 ratio for 2 to 4 hours. The peptide monomer was then filtered, precipitated and centrifuged in cold ether and dried overnight under vacuum. Cleaved monomers and peptides were characterized via analytical HPLC and mass spectrometry and then purified by RP-HPLC. The identity of the peptide monomer was confirmed by ESI-MS or MALDI-ToF MS and purity was verified by observation of a single peak in the analytical RP-HPLC chromatogram.

The purity of the peptide monomer was verified by scale RP-HPLC, where a single peak in the chromatogram of a newly purified peptide monomer was taken as an indication of a pure material. See the high-performance liquid chromatography (HPLC) analytical trace at FIG. 18C. RP-HPLC was performed on a Jupiter Proteo90A Phenomenex column (150×4.60 mm) equipped with a Hitachi-Elite™ LaChrom L2130 pump and a UV-Vis detector (Hitachi-Elite™ LaChrom L-2420) monitoring at 214 nm. The peptide monomer was purified on a preparative-scale Jupiter Proteo90A Phenomenex column (2050×25.0 mm) using an Armen Spot Prep II System and analyzed for purity using a gradient buffer system in which Buffer A is 0.1% TFA in water and Buffer B is 0.1% TFA in acetonitrile.

The identity of the SEQ ID NO: 2 (RQDIDLGVSRR) peptide monomer was confirmed by electrospray ionization (ESI) mass spectrometry with a mass (2⁺) of 787 Da (i.e., a mass of 1574 Da), as evidenced by FIG. 18B.

Polymerization was carried out in in dry, degassed dimethylformamide with 1M LiCl using Grubbs 3rd generation catalyst ((IMesH₂)(C₅H₅N)₂(Cl)₂Ru═CHPh) in equivalent ratios to generate the desired PLPs. For example, a theoretical desired degree of polymerization (DP) of 15 for a SEQ ID NO: 2 (RQDIDLGVSRR) homopolymer, a monomer stock of 30 mM was added to a catalyst stock of 2 mM of equal volume to generate a final reaction solution with 15 equivalents of monomer (15 mM): 1 equivalent of catalyst (1 mM) in 1.2 mL total of dry, degassed 1M LiCl DMF. The reaction was left at room temperature under stirring in a glove box under nitrogen gas. The PLP was recovered via precipitation with cold ether and centrifugation and dried overnight. The PLP was further purified using dialysis in regenerated cellulose (3500 Da pore size) and 2 liters of miliQ water over a 48 hour period. The water was renewed at 24 hours and 48 hours, and the dialyzed materials were collected, sterile filtered using a 0.22-micron PES filter, and lyophilized.

The polymerization reaction was monitored using ¹H NMR spectroscopy by measuring the consumption of the peptide monomer and to determine the time period required to reach completion. Termination was done with ethyl vinyl ether (10 eq) for 30 minutes at room temperature with stirring. FIG. 18D depicts the polymerization reaction of polynorbornene dicarboxyimide-based SEQ ID NO: 2 (RQDIDLGVSRR) peptide monomer prepared according to this example, and the ¹H NMR spectra for the time course experiments monitoring the polymerization reaction. The disappearance of the resonance at 6=6.3 ppm corresponding to the olefin protons of the monomer and the coincident appearance of resonances 6=5-6 ppm, which correspond to the cis and trans olefin protons of the polymer backbone. The kinetics of the polymerization reaction of polynorbornene dicarboxyimide-based SEQ ID NO: 2 (RQDIDLGVSRR) peptide monomer, as determined by the ¹H NMR time course experiment depicted in FIG. 18D are set forth in FIG. 18E.

The molecular weight (Mn) and degree of polymerization was determined by SDS-PAGE. Samples were prepared for SDS-PAGE in miliQ water at a concentration of 1 mg/ml. The sample were added to Laemmli sample buffer at a 2:3 ratio (20 μL buffer:30 μL prepared sample) and then heated at 90° C. for 5 minutes. The samples were then loaded at 30 μL/well into an AnyKD mini Protean TGX Precast Protein Gel along with 10 μL of the Precision Plus Protein Dual Xtra 2-250 kDa ladder. The gel was run in Tris/Gly/SDS Buffer at 120 V until the samples reached the bottom of the gel. The PLP was visualized on the gel using an Instant Coomassie Blue Stain which was applied with shaking at 70 rpm for approximately 15 minutes. Gels were rinsed and imaged for analysis. The results are set forth in FIG. 18F.

The weight average molecular weight (Mw), number average molecular weight (Mn), polydispersity (PD), and degree of polymerization (DP) were determined by aqueous phase gel permeation chromatography (GPC), and the results are set forth in FIG. 18G. To this end, aqueous phase GPC measurements were performed using a TOSOH Biosciences TSKgel G500PW XL-CP column (7.8 mm ID×30 cm, 10 μm) with 0.1 M sodium nitrate buffer containing 0.1% TFA as the mobile phase with a flow rate of 1.0 mL/min. Detection consisted of a Wyatt Optilab T-rEX refractive index detector operating at 658 nm and a Wyatt DAWN® HELEOS® II light scattering detector operating at 659 nm. Absolute molecular weights and polydispersity were calculated using the Wyatt ASTRA software with a dn/dc of 0.185.

As shown in FIG. 18G, the polymerization reaction of polynorbornene dicarboxyimide-based brush homopolymer comprising the peptide SEQ ID NO: 2 (RQDIDLGVSRR) had a weight average molecular weight (Mw) of 24.9 kDa, a number average molecular weight (Mn) of 25.9 kDa, a polydispersity (PD) of 1.042, and a degree of polymerization (DP) of 15.8.

### Example 11

This example provides an exemplary synthesis of a polynorbornene dicarboxyimide-based brush copolymer comprising SEQ ID NO: 2 (RQDIDLGVSRR) (“DLG”) and SEQ ID NO: 150 (LDPETGEFLRRRR) (“ETGE”).

Peptide SEQ ID NO: 150 (LDPETGEFLRRRR) was synthesized via standard Fmoc based solid phase synthesis techniques under Microwave conditions on a CEM Liberty Blue Microwave Peptide Synthesizer. The peptide was prepared using Rink amide MBHA resin (aapptec), or Rink Amide ProTide Resin (CEM) under standard instrument conditions involving Fmoc deprotection with 20% 4-methylpiperidine in DMF, followed by amide coupling of the Fmoc and side chain-protected amino acid, with diisopropylcarbodiimide (DIC) and Oxyma Pure.

The polynorbornene dicarboxyimide-based monomer comprising peptide SEQ ID NO: 150 (LDPETGEFLRRRR) was prepared by amide coupling to N-(hexanoic acid)-cis-5-norbornene-exo-dicarboximide at the N-terminus of peptide SEQ ID NO: 150 (LDPETGEFLRRRR). Following completion of the synthesis, the peptide was cleaved from the resin by treatment with TFA/H₂O/TIPS in a 9.5:2.5:2.5 ratio for 2 to 4 hours. The peptide monomer was then filtered, precipitated and centrifuged in cold ether and dried overnight under vacuum. Cleaved monomers and peptides were characterized via analytical HPLC and mass spectrometry and then purified by RP-HPLC. The identity of the peptide monomer was confirmed by ESI-MS or MALDI-ToF MS and purity was verified by observation of a single peak in the analytical RP-HPLC chromatogram.

Block copolymers having a DLG:ETGE monomer ratio of 8:8, 5:10, and 10:5 were prepared by the following general procedure using the peptide SEQ ID NO: 2 (RQDIDLGVSRR) monomer of Example 10 and the peptide SEQ ID NO: 150 (LDPETGEFLRRRR) of this Example. Polymerization was carried out in in dry, degassed dimethylformamide with 1M LiCl using Grubbs 3rd generation catalyst ((IMesH₂)(C₅H₅N)₂(Cl)₂Ru═CHPh) in equivalent ratios to generate the desired PLPs. For example, a theoretical desired block size of 8 for a SEQ ID NO: 2 (RQDIDLGVSRR) and a theoretical desired block size of 8 for a SEQ ID NO: 150 (LDPETGEFLRRRR), a monomer stock of SEQ ID NO: 2 (RQDIDLGVSRR) at 30 mM was added to a catalyst stock of 3.75 mM of equal volume to generate a final reaction solution with 8 equivalents of monomer (15 mM): 1 equivalent of catalyst (1.88 mM) in 0.55 mL total of dry, degassed 1M LiCl DMF. The reaction was left at room temperature under stirring in a glove box under nitrogen gas until the block A reaction was complete. Additional monomer (15.73 mg in 0.11 ml, 75 mM) of block B (SEQ ID NO: 150 (LDPETGEFLRRRR) was added at 8 equivalents to the 1 equivalent of catalyst in the reaction vessel. The reaction for the addition of block B was carried out until the reaction was complete by NMR. The polymerization was terminated using excess ethyl vinyl ether for 30 min with stirring. The PLP was recovered via precipitation with cold ether and centrifugation and dried overnight. The PLP was further purified using dialysis in regenerated cellulose (3500 Da pore size) and 2 liters of miliQ water over a 48 hour period. The water was renewed at 24 hours and 48 hours, and the dialyzed materials were collected, sterile filtered using a 0.22-micron PES filter, and lyophilized.

For example, a theoretical desired block size of 5 for a SEQ ID NO: 2 (RQDIDLGVSRR) and a theoretical desired block size of 10 for a SEQ ID NO: 150 (LDPETGEFLRRRR), a monomer stock of SEQ ID NO: 2 (RQDIDLGVSRR) at 30 mM was added to a catalyst stock of 4.37 mM of equal volume to generate a final reaction solution with 5 equivalents of monomer (15 mM): 1 equivalent of catalyst (3 mM) in 0.55 mL total of dry, degassed 1M LiCl DMF. The reaction was left at room temperature under stirring in a glove box under nitrogen gas until the block A reaction was complete. Additional monomer (31.47 mg in 0.22 ml, 75 mM) of block B (SEQ ID NO: 150 (LDPETGEFLRRRR) was added at 10 equivalents to the 1 equivalent of catalyst in the reaction vessel. The reaction for the addition of block B was carried out until the reaction was complete by NMR. The polymerization was terminated using excess ethyl vinyl ether for 30 min with stirring. The PLP was recovered via precipitation with cold ether and centrifugation and dried overnight. The PLP was further purified using dialysis in regenerated cellulose (3500 Da pore size) and 2 liters of miliQ water over a 48 hour period. The water was renewed at 24 hours and 48 hours, and the dialyzed materials were collected, sterile filtered using a 0.22-micron PES filter, and lyophilized.

For example, a theoretical desired block size of 10 for a SEQ ID NO: 2 (RQDIDLGVSRR) and a theoretical desired block size of 5 for a SEQ ID NO: 150 (LDPETGEFLRRRR), a monomer stock of SEQ ID NO: 2 (RQDIDLGVSRR) at 30 mM was added to a catalyst stock of 3 mM of equal volume to generate a final reaction solution with 10 equivalents of monomer (15 mM): 1 equivalent of catalyst (1.5 mM) in 0.55 mL total of dry, degassed 1M LiCl DMF. The reaction was left at room temperature under stirring in a glove box under nitrogen gas until the block A reaction was complete. Additional monomer (7.87 mg in 0.11 ml, 37 mM) of block B (SEQ ID NO: 150 (LDPETGEFLRRRR) was added at 5 equivalents to the 1 equivalent of catalyst in the reaction vessel. The reaction for the addition of block B was carried out until the reaction was complete by NMR. The polymerization was terminated using excess ethyl vinyl ether for 30 min with stirring. The PLP was recovered via precipitation with cold ether and centrifugation and dried overnight. The PLP was further purified using dialysis in regenerated cellulose (3500 Da pore size) and 2 liters of miliQ water over a 48 hour period. The water was renewed at 24 hours and 48 hours, and the dialyzed materials were collected, sterile filtered using a 0.22-micron PES filter, and lyophilized.

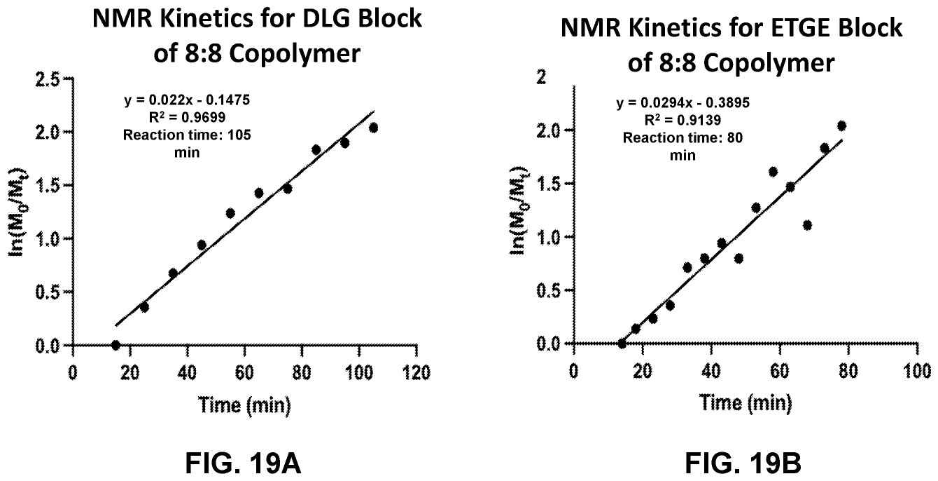

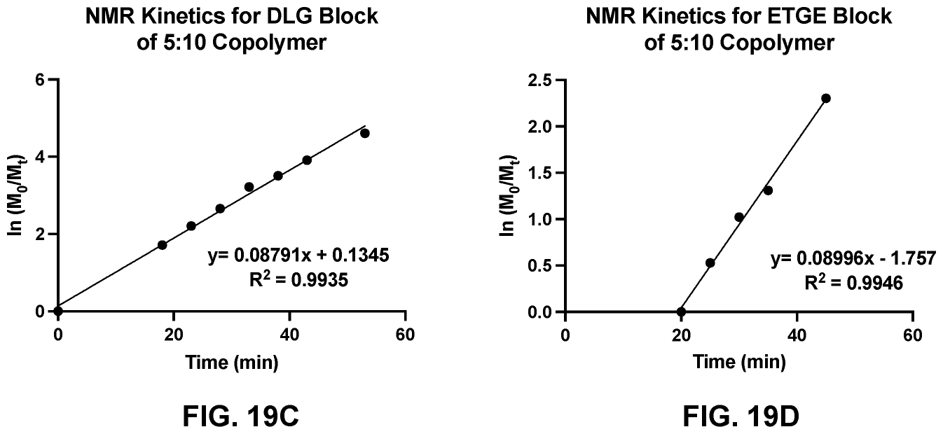

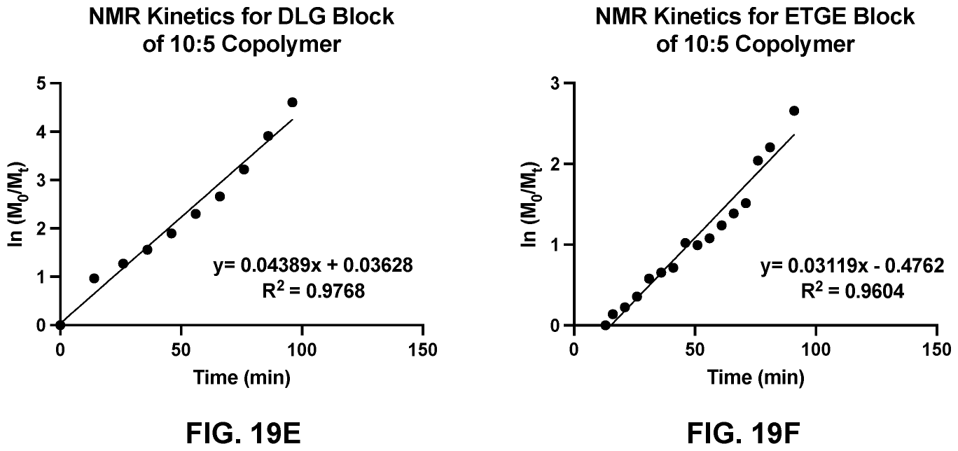

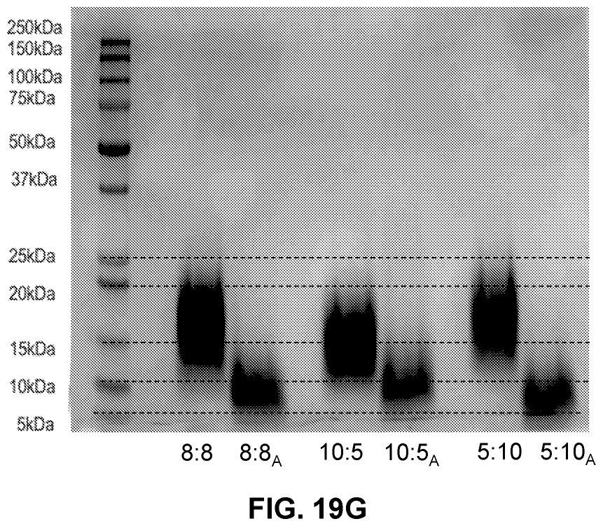

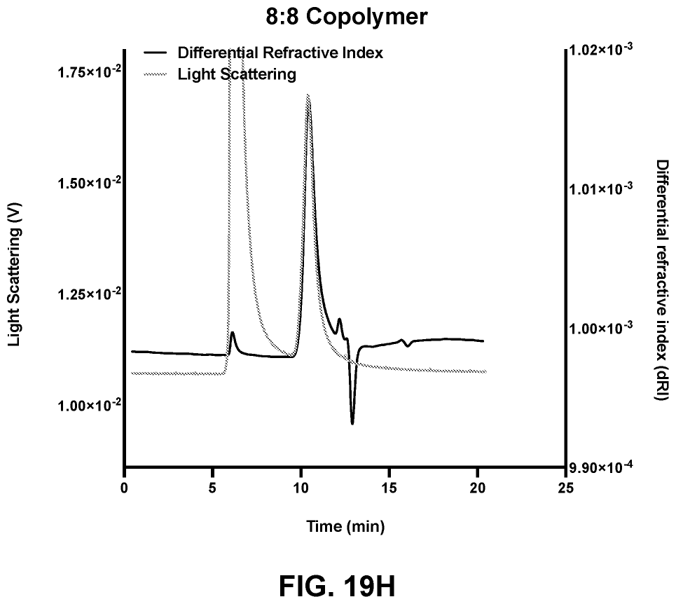

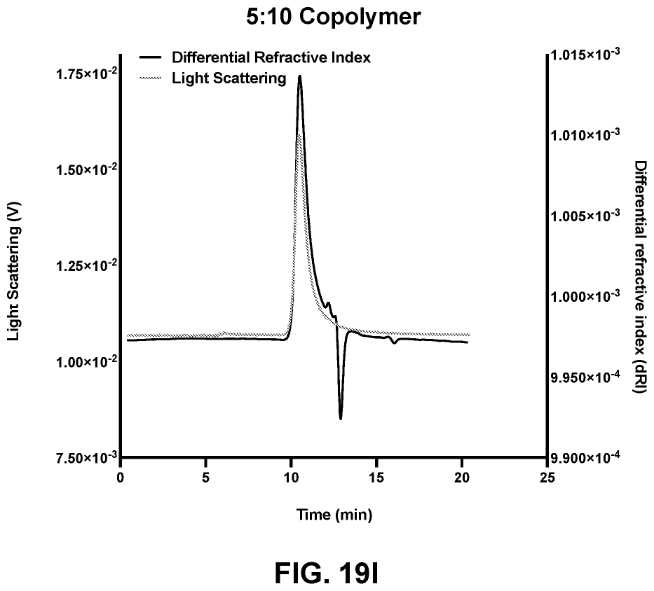

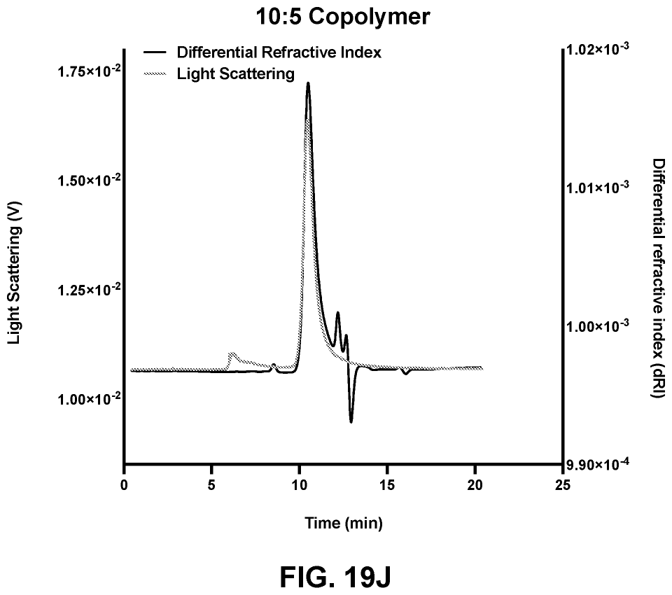

The polymerization reactions of the DLG block and the ETGE block of the copolymers having a DLG:ETGE monomer ratio of 8:8, 5:10, and 10:5 were monitored using ¹H NMR spectroscopy by measuring the consumption of the peptide monomer and to determine the time period required for each block to reach completion. Termination was done with ethyl vinyl ether (10 eq) for 30 minutes at room temperature with stirring. FIGS. 19A and 19B show the rate kinetics for the DLG block (FIG. 19A) and the ETGE block (FIG. 19B), of the polynorbornene dicarboxyimide-based brush 8:8 copolymer, FIGS. 19C and 19D show the rate kinetics for the DLG block (FIG. 19C) and the ETGE block (FIG. 19D) of the polynorbornene dicarboxyimide-based brush 5:10 copolymer, and FIGS. 19E and 19F show the rate kinetics for the DLG block (FIG. 19E) and the ETGE block (FIG. 19F) of the polynorbornene dicarboxyimide-based brush 10:5 copolymer.

The number average molecular weight (Mn) and degree of polymerization was determined by SDS-PAGE. Samples were prepared for SDS-PAGE in miliQ water at a concentration of 1 mg/ml. The samples were added to Laemmli sample buffer at a 2:3 ratio (20 μL buffer:30 μL prepared sample) and then heated at 90° C. for 5 minutes. The samples were then loaded at 30 μL/well into an AnyKD mini Protean TGX Precast Protein Gel along with 10 μL of the Precision Plus Protein Dual Xtra 2-250 kDa ladder. The gel was run in Tris/Gly/SDS Buffer at 120 V until the samples reached the bottom of the gel. The PLPs were visualized on the gel using an Instant Coomassie Blue Stain which was applied with shaking at 70 rpm for approximately 15 minutes. The gels were rinsed and imaged for analysis, and the results of the copolymers and DLG block (“A”) are set forth in FIG. 19G.

The weight average molecular weight (Mw), number average molecular weight (Mn), polydispersity (PD), and degree of polymerization (DP) were determined by aqueous phase gel permeation chromatography (GPC) for the copolymers having a DLG:ETGE monomer ratio of 8:8, 5:10, and 10:5. To this end, aqueous phase GPC measurements were performed using a TOSOH Biosciences TSKgel G50OPW XL-CP column (7.8 mm ID×30 cm, 10 μm) with 0.1 M sodium nitrate buffer containing 0.1% TFA as the mobile phase with a flow rate of 1.0 mL/min. Detection consisted of a Wyatt Optilab T-rEX refractive index detector operating at 658 nm and a Wyatt DAWN® HELEOS® II light scattering detector operating at 659 nm. Absolute molecular weights and polydispersity were calculated using the Wyatt ASTRA software with a dn/dc of 0.185. The differential refractive index and light scattering for the copolymers having a DLG:ETGE monomer ratio of 8:8, 5:10, and 10:5 are set forth in FIGS. 19H-19J, respectively.

Using the results set forth in FIGS. 19G-19J, the copolymers having a DLG:ETGE monomer ratio of 8:8, 5:10, and 10:5 had a weight average molecular weight (Mw), a number average molecular weight (Mn), a polydispersity (PD), and a degree of polymerization (DP), as set forth in Table 2.

TABLE 2
Characterization of the DLG-ETGE Copolymers

| | Expected | M_{w} | Mₙ | | SDS-Page |
|---|---|---|---|---|---|
| Polymer Ratio | (Daltons) | (Daltons) | (Daltons) | PDI | (Daltons) |
| 8:8 | 27,832 | 29,590 | 28,200 | 1.049 | 19,500 |
| 8:8 Block A | 12,592 | — | — | — | 10,000 |
| 5:10 | 26,920 | 23,810 | 23,540 | 1.012 | 20,500 |
| 5:10 Block A | 7,870 | — | — | — | 8,000 |
| 10:5 | 25,265 | 20,560 | 20,250 | 1.015 | 17,000 |
| 10:5 Block A | 15,740 | — | — | — | 12,000 |

### Example 12

This example shows the biocompatibility of a protein-like polymer described herein, as determined by the cell viability of Antioxidant Response Element (ARE) Luciferase HepG2 Reporter cells treated with a polynorbornene dicarboxyimide-based SEQ ID NO: 2 (RQDIDLGVSRR) polymer. An MTS cell viability assay was performed over a concentration range of 0 μM to 100 μM for the polynorbornene dicarboxyimide-based SEQ ID NO: 2 (RQDIDLGVSRR) polymer (i.e., DLG Homopolymer).

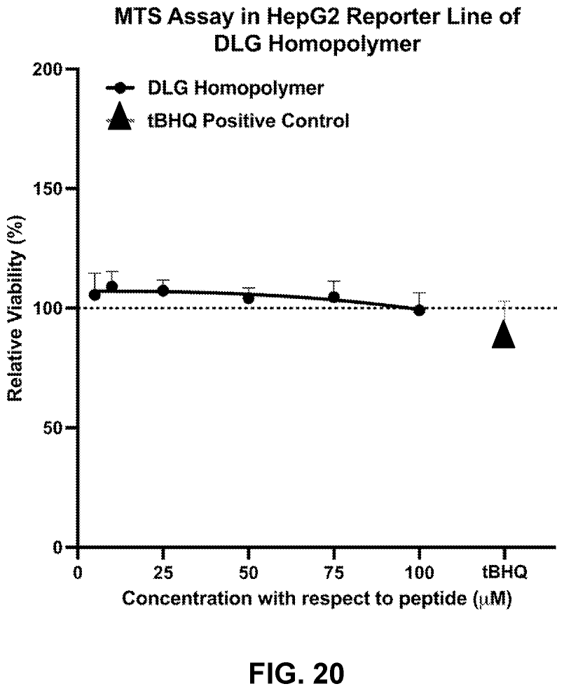

Cells were plated at a seeding density of 25k cells/well into 96 well plates in cell growth media and allowed to incubate for 24 hours. Cells were treated with the polynorbornene dicarboxyimide-based SEQ ID NO: 2 (RQDIDLGVSRR) polymer over a range of concentrations (i.e., 0 μM to 100 μM with respect to peptide) at n=4. After 24 hours, 10 μL of MTS reagent was added to each well, and the cells incubated for four hours at 37° C. Absorbance was measured at 490 nm using a Perkin Elmer EnSpire plate reader every hour after MTS addition. Viability was assessed after background subtraction from cell free control wells and calculated as relative viability based on the average of control wells. Viability is reported as a percentage of control groups and prepared for presentation using Prism9. The results are set forth in FIG. 20.

As shown in FIG. 20, the MTS assay shows that the polynorbornene dicarboxyimide-based SEQ ID NO: 2 (RQDIDLGVSRR) polymer (i.e., DLG Homopolymer) maintained approximately 100% relative viability. These results further demonstrate that that the polynorbornene dicarboxyimide-based SEQ ID NO: 2 (RQDIDLGVSRR) polymer (i.e., DLG Homopolymer) is well tolerated.

### Example 13

This Example shows the Antioxidant Response Element (ARE) activation and parallel cell viability of a protein-like polymer described herein, as determined by a reporter assay of Antioxidant Response Element (ARE) Luciferase HepG2 Reporter cells treated with a polynorbornene dicarboxyimide-based brush homopolymer (degree of polymerization=15) with SEQ ID NO: 150 (LDPETGEFLRRRR) (“ETGE”), a polynorbornene dicarboxyimide-based brush homopolymer (degree of polymerization=15) with SEQ ID NO: 2 (RQDIDLGVSRR) (“DLG”), and a polynorbornene dicarboxyimide-based brush copolymer (“DLG:ETGE”) (degree of polymerization=16). The reporter and cell viability assays were performed over a concentration range of 0 μM to 10 μM for each of the polymers described.

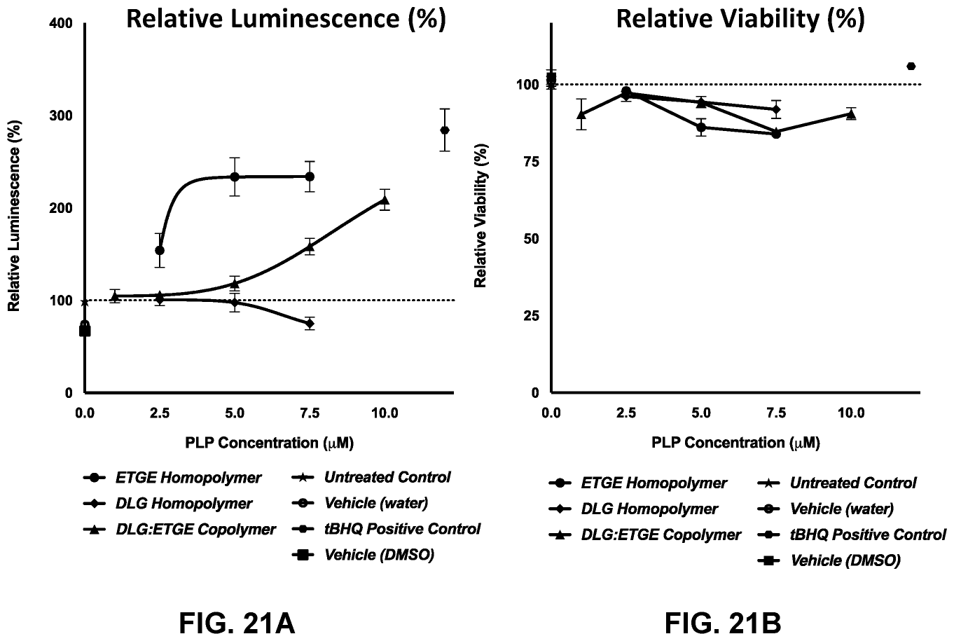

Cells were plated at a seeding density of 25k cells/well into 96 well plates in cell growth media and allowed to incubate for 24 hours. Cells were treated with the polymers over a range of concentrations (i.e., 0 μM to 10 μM) at n=4. For the reporter assay, cells were incubated for 24 hours post-treatment. ONE-step luciferase assay reagent was added at 100 μL/well, followed by rocking at room temperature for 15 min. Luminescence was assessed using the Biotek SynergyNeo2 plate reader at the High Throughput Analysis Core at Northwestern University. Background luminescence of cell free controls wells was subtracted from test wells. ARE activation is reported as luminescence relative to the average of the untreated control and vehicle control wells, and the result are set forth in FIG. 21A. For the viability assay, after 24 hours, 10 μL of MTS reagent was added to each well, and the cells incubated for four hours at 37° C. Absorbance was measured at 490 nm using a Perkin Elmer EnSpire plate reader every hour after MTS addition. Viability was assessed after background subtraction from cell free control wells and calculated as relative viability based on the average of control wells. Viability is reported as a percentage of control groups and prepared for presentation using Prism9. The results are set forth in FIG. 21B.

As demonstrated by FIG. 21A, the reporter assay shows that while the ETGE homopolymer is most activating, the DLG homopolymer does not activate alone. However, a copolymer incorporating both domains has modulated activation. The MTS assay (FIG. 21B) shows that the polynorbornene dicarboxyimide-based brush homopolymer with SEQ ID NO: 150 (LDPETGEFLRRRR), the polynorbornene dicarboxyimide-based brush homopolymer with SEQ ID NO: 2 (RQDIDLGVSRR), and the polynorbornene dicarboxyimide-based brush copolymer with SEQ ID NO: 150 (LDPETGEFLRRRR) and SEQ ID NO: 2 (RQDIDLGVSRR) maintained approximately 100% relative viability. These results further demonstrate that the polymers are well tolerated.

### Example 14

This example provides the results of a fluorescence polarization Keap1 binding assay, as measured by percent FAM-Nrf2 binding, exhibited by a polynorbornene dicarboxyimide-based brush homopolymer (degree of polymerization=15) with SEQ ID NO: 150 (LDPETGEFLRRRR) (“ETGE”) and a polynorbornene dicarboxyimide-based brush homopolymer (degree of polymerization=15) with SEQ ID NO: 2 (RQDIDLGVSRR) (“DLG”).

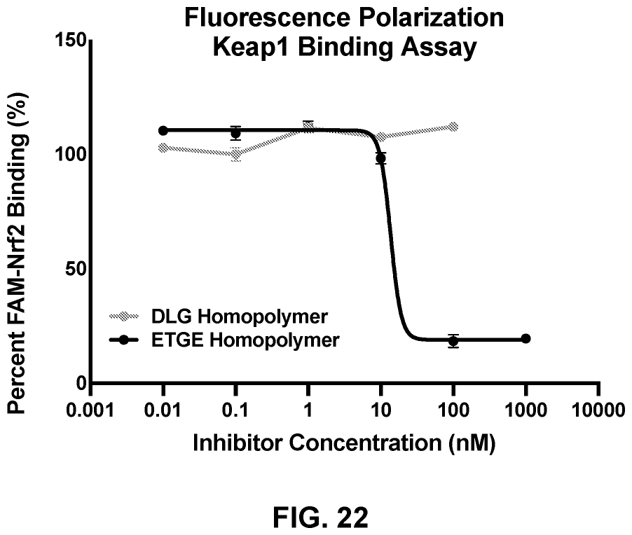

The polynorbornene dicarboxyimide-based brush homopolymers were assessed for Keap1/Nrf2 disruption using a competitive inhibition screening assay that uses labeled Nrf2 peptide (FAM-LDEETGEFL) and human recombinant Keap1 (BPS bioscience, San Diego, CA). The assay was carried out according to established protocols. Briefly, test inhibitors (n=3) were added at varying concentrations to wells of a black 96-well plate along with assay buffer, BSA, FAM-Nrf2, and Keap1 and allowed to incubate for 30 minutes at room temperature. The fluorescence polarization (excitation 475-495 nm, emission 518-538 nm) of the samples were measured using the Biotek SynergyNeo2 plate reader at the High Throughput Analysis Core at Northwestern University. Blank control was assay buffer and inhibitor vehicle. Nrf2 negative binding control included assay buffer, BSA, FAM-Nrf2, and inhibitor vehicle. Nrf2 positive binding control included assay buffer, BSA, FAM-Nrf2, inhibitor vehicle and Keap1. Data was assessed for IC₅₀ values by quantifying percent Nrf2 activity relative to inhibitor concentration by fitting to a nonlinear model for a dose response (absolute IC₅₀) using Prism 9. The results for the polynorbornene dicarboxyimide-based brush homopolymer with SEQ ID NO: 150 (LDPETGEFLRRRR) and the polynorbornene dicarboxyimide-based brush homopolymer with SEQ ID NO: 2 (RQDIDLGVSRR) are set forth in FIG. 22.

As demonstrated by FIG. 22, the polynorbornene dicarboxyimide-based brush homopolymer with SEQ ID NO: 2 (RQDIDLGVSRR) alone does not out compete the labeled Nrf2 peptide (FAM-LDEETGEFL) at concentrations less than 100 nM. In contrast, the polynorbornene dicarboxyimide-based brush homopolymer with SEQ ID NO: 150 (LDPETGEFLRRRR) out competes the labeled Nrf2 peptide (FAM-LDEETGEFL) at concentrations greater than 10 nM.

### Example 15

This example provides the results of a time-resolved fluorescence resonance energy transfer (TR-FRET) assay for a polynorbornene dicarboxyimide-based brush homopolymer (degree of polymerization=15) with SEQ ID NO: 150 (LDPETGEFLRRRR) (“ETGE”) and a polynorbornene dicarboxyimide-based brush copolymer (“DLG:ETGE”) (degree of polymerization=16).

The time-resolved fluorescence resonance energy transfer (TR-FRET) assay was performed as described previously Colarusso et al. (Bioorganic Med. Chem., 28; 1-12 (2020)). Briefly, a Kelch domain labeled with biotin was bound to streptavidin-europium energy donor. The LDEETGEFL peptide was labeled with the energy acceptor, Alexa-Fluor647 (AF647), and acted as a ligand of the Kelch domain. When the labeled peptide was allowed to interact with the labeled Kelch domain, an energy transfer from the donor to acceptor fluorophores resulted in a TR-FRET signal. However, in the presence of an inhibitor, the TR-FRET signal decreases.

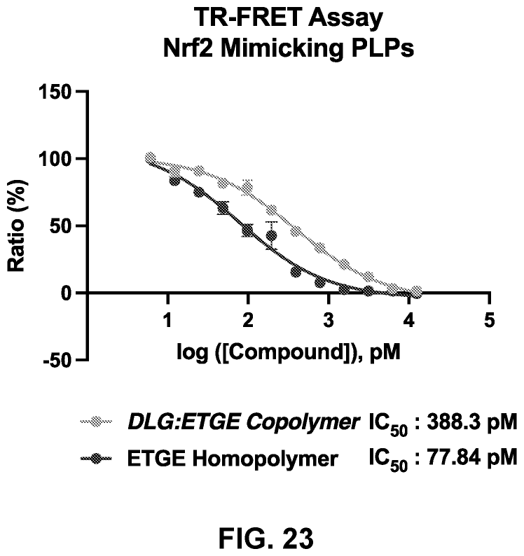

The results of the time-resolved fluorescence resonance energy transfer (TR-FRET) assay for the polynorbornene dicarboxyimide-based brush homopolymer with SEQ ID NO: 150 (LDPETGEFLRRRR) and the polynorbornene dicarboxyimide-based brush copolymer with SEQ ID NO: 150 (LDPETGEFLRRRR) and SEQ ID NO: 2 (RQDIDLGVSRR) are set forth in FIG. 23.

As is apparent from the results set forth in FIG. 23, the polynorbornene dicarboxyimide-based brush copolymer with SEQ ID NO: 150 (LDPETGEFLRRRR) and SEQ ID NO: 2 (RQDIDLGVSRR) is capable of inhibiting Keap1 with an IC₅₀ of 388.3 μM, whereas the polynorbornene dicarboxyimide-based brush homopolymer with SEQ ID NO: 150 (LDPETGEFLRRRR) is capable of inhibiting Keap1 with an IC₅₀ of 77.84 μM.

### Example 16

This example provides the results of a fluorescence polarization Keap1 binding assay, as measured by percent FAM-Nrf2 binding, exhibited by a polynorbornene dicarboxyimide-based brush homopolymer (degree of polymerization=15) with SEQ ID NO: 150 (LDPETGEFLRRRR) (“ETGE”) and polynorbornene dicarboxyimide-based brush copolymers of SEQ ID NO: 150 (LDPETGEFLRRRR) (“ETGE”) with SEQ ID NO: 2 (RQDIDLGVSRR) (“DLG”) at ratios of DLG:ETGE of 5:10, 8:8; and 10:5, as prepared in Example 11.

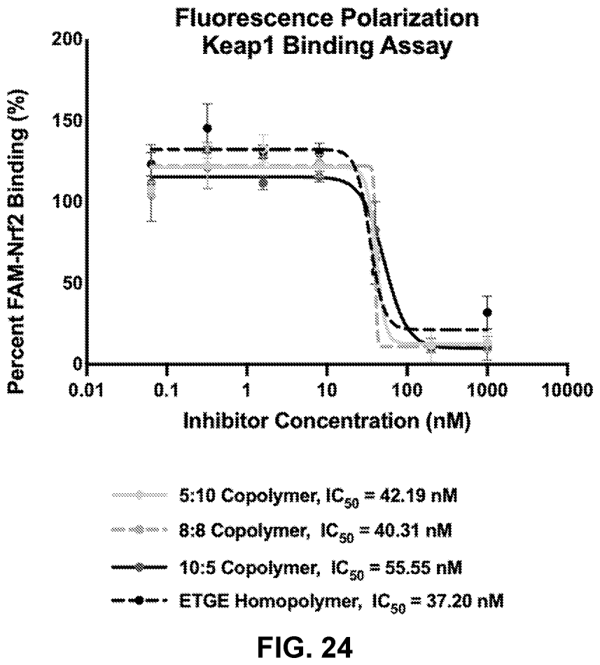

The polynorbornene dicarboxyimide-based brush homopolymer and copolymers were assessed for Keap1/Nrf2 disruption using a competitive inhibition screening assay that uses labeled Nrf2 peptide (FAM-LDEETGEFL) and human recombinant Keap1 (BPS bioscience, San Diego, CA). The assay was carried out according to established protocols. Briefly, test inhibitors (n=3) were added at varying concentrations to wells of a black 96-well plate along with assay buffer, BSA, FAM-Nrf2, and Keap1 and allowed to incubate for 30 minutes at room temperature. The fluorescence polarization (excitation 475-495 nm, emission 518-538 nm) of the samples were measured using the Biotek SynergyNeo2 plate reader at the High Throughput Analysis Core at Northwestern University. Blank control was assay buffer and inhibitor vehicle. Nrf2 negative binding control included assay buffer, BSA, FAM-Nrf2, and inhibitor vehicle. Nrf2 positive binding control included assay buffer, BSA, FAM-Nrf2, inhibitor vehicle and Keap1. Data was assessed for IC₅₀ values by quantifying percent Nrf2 activity relative to inhibitor concentration by fitting to a nonlinear model for a dose response (absolute IC₅₀) using Prism 9. The results for the polynorbornene dicarboxyimide-based brush homopolymer (degree of polymerization=15) with SEQ ID NO: 150 (LDPETGEFLRRRR) (“ETGE”) and polynorbornene dicarboxyimide-based brush copolymers of SEQ ID NO: 150 (LDPETGEFLRRRR) (“ETGE”) with SEQ ID NO: 2 (RQDIDLGVSRR) (“DLG”) at ratios of DLG:ETGE of 5:10, 8:8; and 10:5 are set forth in FIG. 24.

As demonstrated by FIG. 24, all of the polymers tested showed evidence of Keap1-inhibition.

### Example 17

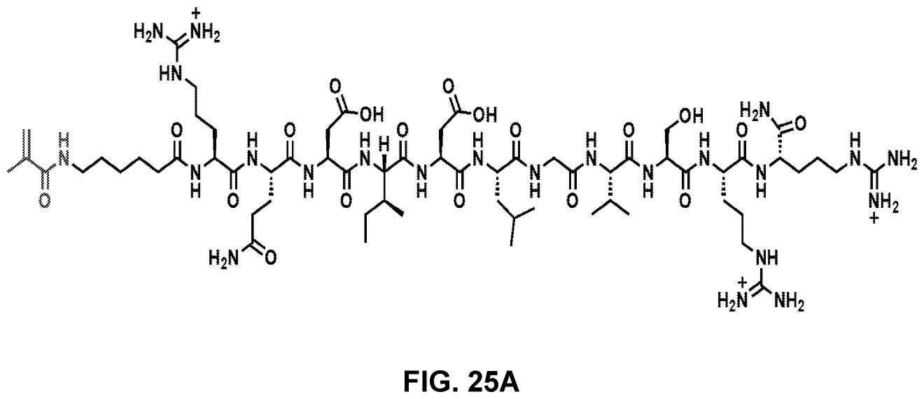

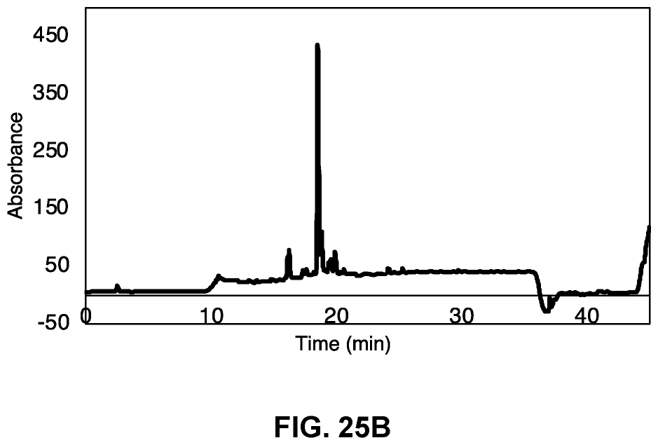

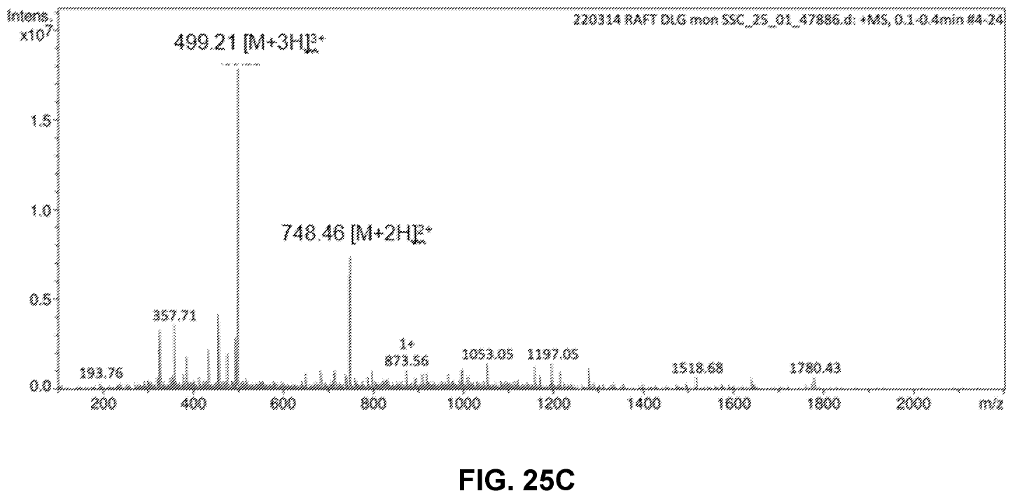

This example provides an exemplary synthesis of a methacrylamide-based brush polymer comprising the peptide SEQ ID NO: 2 (RQDIDLGVSRR), as depicted in FIG. 25A.

Peptide SEQ ID NO: 2 (RQDIDLGVSRR) was synthesized via solid phase peptide synthesis on Rink amide MBHA resin using an AAPPTec Focus XC automated synthesizer. Fluorenylmethyloxycarbonyl (Fmoc)-6-aminohexanoic acid (3.0 eq.), HBTU (2.9 eq), and N,N-diisopropylethylamine (DIPEA) (6.0 eq.) in 12 mL of dimethylformamide (DMF) were added to peptide on resin and placed on shaker for 2 hrs. The resin was isolated via vacuum filtration and washed 2× with fresh DMF. Care was taken to ensure resin was never completely dried.

Next, the Fmoc group was removed by adding 15 mL of 20% piperidine in DMF to the resin and placed on shaker for 20 minutes. The solvent was filtered off by vacuum filtration and resin washed 2× with DMF. Another 15 mL of 20% piperidine in DMF was added to resin and placed on shaker for 10 minutes. The solvent was filtered off by vacuum filtration and resin washed 2× with DMF. Finally, DMF (15 mL) was added to resin, funnel capped and briefly hand shook to ensure all piperidine had been removed, then filtered off.

Methacrylic acid (3.0 eq.), HBTU (2.9 eq.), and DIPEA (6.0 eq.) in 12 mL of DMF were added to the peptide on resin and placed on shaker for 2 hrs. The resin was isolated via vacuum filtration and washed with DMF then dichloromethane (DCM) twice. The resin was dried completely then placed in desiccator overnight to yield the methacrylamide monomer comprising SEQ ID NO: 2 (RQDIDLGVSRR) on resin.

Following completion of the synthesis, the methacrylamide monomer comprising SEQ ID NO: 2 (RQDIDLGVSRR) was cleaved from Rink resin with a cleavage cocktail composed of 88% trifluoroacetic acid (TFA), 2% triisopropylsilane (TIPS), 5% 3,6-dioxa-1,8-octanedithiol (DODT), and 5% milli-Q water. The monomer on resin was added to cleavage cocktail at a ratio of 500 mg resin:10 mL cleavage cocktail, and the resulting solution was stirred for 3 hours. The resin was filtered off and washed with dichloromethane (DCM). The monomer solution was collected in a falcon tube and the solution was left to evaporate under a stream of nitrogen gas until approximately 2 mL remained. Cold ethyl ether (40 mL) was added to the monomer solution and monomer crashed out. Solution was vortexed then centrifuged at 10,000 rpm for 10 minutes at 4° C. The ether was decanted off, and another 40 mL of fresh ether was added to monomer. The solution was vortexed then centrifuged again under the same conditions. This step was repeated one more time for a total of 3 centrifuge runs, and after the last run, the ether was decanted off and cleaved monomer was placed in the desiccator overnight.

The purity of the peptide monomer was verified by scale RP-HPLC, where a single peak in the chromatogram of a newly purified peptide monomer was taken as an indication of a pure material. See the high-performance liquid chromatography (HPLC) analytical trace at FIG. 25B. RP-HPLC was performed on a Jupiter Proteo90A Phenomenex column (150×4.60 mm) equipped with a Hitachi-Elite™ LaChrom L2130 pump and a UV-Vis detector (Hitachi-Elite™ LaChrom L-2420) monitoring at 214 nm. The peptide monomer was purified on a preparative-scale Jupiter Proteo90A Phenomenex column (2050×25.0 mm) using an Armen Spot Prep II System and analyzed for purity using a gradient buffer system in which Buffer A is 0.1% TFA in water and Buffer B is 0.1% TFA in acetonitrile.

After cleavage, the methacrylamide monomer comprising SEQ ID NO: 2 (RQDIDLGVSRR) was purified with reverse phase preparatory HPLC. A 3 mg/mL solution of the methacrylamide monomer comprising SEQ ID NO: 2 (RQDIDLGVSRR) was made in 85% buffer A (99.9% milli-Q water, 0.1% TFA), 15% buffer B (99.9% ACN, 0.1% TFA) and run on a gradient of 15-35% buffer B over 30 minutes. The injection volume was 20 mL and the peak eluted at 18 minutes with an absorbance at 214 nm was collected and lyophilized to yield the purified methacrylamide monomer comprising SEQ ID NO: 2 (RQDIDLGVSRR).

The identity of the methacrylamide monomer comprising SEQ ID NO: 2 (RQDIDLGVSRR) was confirmed by ESI mass spectrometry with a mass [M+2H]²⁺ of 748 Da (i.e., a mass of 1497 Da), as evidenced by FIG. 25C.

Polymerizations targeting a theoretical degree of polymerization of 15 are carried out by dissolving purified methacrylamide monomer comprising SEQ ID NO: 2 (RQDIDLGVSRR) (0.011 mmol, 15 eq.) in 128 μL of 25% acetate buffer (1 M, pH=5) and 75% miliQ water (32 μL acetate buffer, 96 μL miliQ water). To the resulting solution is added 4-(((2 carboxyethyl)thio)carbonothioyl)thio 4-cyano pentanoic acid (RAFT agent, RA, 11.2 μL, 20 mg/mL stock in DMSO, 0.00073 mmol, 1 eq.) and lithium phenyl-2,4,6-trimethylbenzoylphosphinate (LPTP, 10.7 μL, 6 mg/mL stock in miliQ water, 0.00022 mmol, 0.3 eq.) for a total reaction volume of 150 μL. The reaction is degassed by purging with nitrogen for 30 minutes and the resulting mixture is placed in an UV light box (365 nm) for 24 hours. After 24 hours, the vial is removed from UV light and the reaction product is confirmed by ¹H NMR spectroscopy.

The exemplary polymerization procedure above targets a theoretical DP of 15 based on the monomer:RA molar ratio of 15:1; however, by changing the monomer:RA molar ratio the theoretical degree of polymerization can be altered.

### STATEMENTS REGARDING INCORPORATION BY REFERENCE AND VARIATIONS

(1) All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).
(2) The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.
(3) When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.
(4) It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”
(5) Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. Nothing herein is to be construed as an admission that the invention is not entitled to antedate such disclosure by virtue of prior invention.
(6) Whenever a range is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.
(7) As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.
(8) One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.
(9) All references, including publications, patent applications, and patents, cited herein are hereby incorporated by reference to the same extent as if each reference were individually and specifically indicated to be incorporated by reference and were set forth in its entirety herein.
(10) The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.
(11) Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A peptide having a sequence having 75% or greater sequence identity with a 6 to 10 amino acid fragment of SEQ ID NO: 218 (MDLIDILWRQDIDLGVSREVFDFS), wherein the peptide further comprises a charge modulating domain having from 1 to 7 amino acid residues.

2. The peptide of claim 1, wherein the peptide comprises a sequence having 85% or greater sequence identity with a 6 to 10 amino acid fragment of SEQ ID NO: 218 (MDLIDILWRQDIDLGVSREVFDFS).

3. The peptide of claim 1, wherein the peptide comprises a 6 to 10 amino acid fragment of SEQ ID NO: 218 (MDLIDILWRQDIDLGVSREVFDFS).

4. The peptide of claim 1, where the peptide has from 11 to 17 amino acid residues comprising a sequence having 85% or greater sequence identity of SEQ ID NO: 1 (RQDIDLGVSR), SEQ ID NO: 220 (ILWRQDIDLGVSR), or SEQ ID NO: 222 (LWRQDIDLGVSR), wherein the peptide further comprises a charge modulating domain having from 1 to 7 amino acid residues.

5. The peptide of claim 4, wherein the sequence having 85% or greater sequence identity of SEQ ID NO: 1 (RQDIDLGVSR), SEQ ID NO: 220 (ILWRQDIDLGVSR), or SEQ ID NO: 222 (LWRQDIDLGVSR) is SEQ ID NO: 1 (RQDIDLGVSR), SEQ ID NO: 220 (ILWRQDIDLGVSR), or SEQ ID NO: 222 (LWRQDIDLGVSR).

6. The peptide of any one of claims 1-5, wherein the charge modulating domain is a glycine-serine domain, a cationic residue domain, or a combination thereof.

7. The peptide of claim 6, wherein the cationic residue domain is from 1 to 7 amino acid residues consisting of lysine, arginine, histidine, or a combination thereof.

8. The peptide of any one of claims 1-7, wherein the peptide has a net positive charge.

9. The peptide of any one of claims 1-8, wherein the peptide has 11 to 14 amino acid residues.

10. The peptide of any one of claims 4-9, wherein the peptide comprises a sequence having 85% or greater sequence identity of SEQ ID NO: 1 (RQDIDLGVSR).

11. The peptide of claim 10, wherein the sequence having 85% or greater sequence identity of SEQ ID NO: 1 (RQDIDLGVSR) is SEQ ID NO: 1 (RQDIDLGVSR).

12. The peptide of any one of claims 1-11, wherein the peptide is
SEQ ID NO: 2
(RQDIDLGVSRR)
SEQ ID NO: 3
(RRQDIDLGVSR)
SEQ ID NO: 4
(RQDIDLGVSRK)
SEQ ID NO: 5
(KRQDIDLGVSR)
SEQ ID NO: 6
(KRQDIDLGVSRR)
SEQ ID NO: 7
(RRQDIDLGVSRK)
SEQ ID NO: 8
(RQDIDLGVSRRR)
SEQ ID NO: 9
(RQDIDLGVSRRRR)
SEQ ID NO: 10
(RQDIDLGVSRRRRR)
SEQ ID NO: 11
(RQDIDLGVSRRRRRR)
SEQ ID NO: 12
(RRRQDIDLGVSR)
SEQ ID NO: 13
(RRRRQDIDLGVSR)
SEQ ID NO: 14
(RRRRRQDIDLGVSR)
SEQ ID NO: 15
(RRRRRRQDIDLGVSR)
SEQ ID NO: 16
(RRRQDIDLGVSRRR)
SEQ ID NO: 17
(RRRRQDIDLGVSRRRR)
SEQ ID NO: 18
(RRQDIDLGVSRR)
SEQ ID NO: 19
(RRQDIDLGVSRRR)
SEQ ID NO: 20
(RRRQDIDLGVSRR)
SEQ ID NO: 21
(RRQDIDLGVSRRRR)
SEQ ID NO: 22
(RRRRQDIDLGVSRR)
SEQ ID NO: 23
(RRQDIDLGVSRRRRR)
SEQ ID NO: 24
(RRRQDIDLGVSRRRR)
SEQ ID NO: 25
(RRRRQDIDLGVSRRR)
SEQ ID NO: 26
(RRRRRQDIDLGVSRR)
SEQ ID NO: 27
(RRQDIDLGVSRRRRRR)
SEQ ID NO: 28
(RRRQDIDLGVSRRRRR)
SEQ ID NO: 29
(RRRRRQDIDLGVSRRR)
SEQ ID NO: 30
(RRRRRRQDIDLGVSRR)
SEQ ID NO: 31
(RQDIDLGVSRKK)
SEQ ID NO: 32
(RQDIDLGVSRKKK)
SEQ ID NO: 33
(RQDIDLGVSRKKKK)
SEQ ID NO: 34
(RQDIDLGVSRKKKKK)
SEQ ID NO: 35
(KKRQDIDLGVSR)
SEQ ID NO: 36
(KKKRQDIDLGVSR)
SEQ ID NO: 37
(KKKKRQDIDLGVSR)
SEQ ID NO: 38
(KKKKKRQDIDLGVSR)
SEQ ID NO: 39
(KKRQDIDLGVSRKK)
SEQ ID NO: 40
(KKKRQDIDLGVSRKKK)
SEQ ID NO: 41
(KRQDIDLGVSRK)
SEQ ID NO: 42
(KRQDIDLGVSRKK)
SEQ ID NO: 43
(KKRQDIDLGVSRK)
SEQ ID NO: 44
(KRQDIDLGVSRKKK)
SEQ ID NO: 45
(KKKRQDIDLGVSRK)
SEQ ID NO: 46
(KRQDIDLGVSRKKKK)
SEQ ID NO: 47
(KKRQDIDLGVSRKKK)
SEQ ID NO: 48
(KKKRQDIDLGVSRKK)
SEQ ID NO: 49
(KKKKRQDIDLGVSRK)
SEQ ID NO: 50
(KRQDIDLGVSRKKKKK)
SEQ ID NO: 51
(KKRQDIDLGVSRKKKK)
SEQ ID NO: 52
(KKKKRQDIDLGVSRKK)
SEQ ID NO: 53
(KKKKKRQDIDLGVSRK)
SEQ ID NO: 54
(RQDIDLGVSRKRKR)
SEQ ID NO: 55
(KRKRRQDIDLGVSR)
SEQ ID NO: 56
(RKRKRQDIDLGVSR)
SEQ ID NO: 57
(RQDIDLGVSRRKRK)
SEQ ID NO: 58
(KKRQDIDLGVSRRR)
SEQ ID NO: 59
(RRRQDIDLGVSRKK)
SEQ ID NO: 60
(KRQDIDLGVSRRRR)
SEQ ID NO: 61
(KKKRQDIDLGVSRR)
SEQ ID NO: 62
(RRRRQDIDLGVSRK)
SEQ ID NO: 63
(KRRQDIDLGVSRKR)
SEQ ID NO: 64
(RKRQDIDLGVSRRK)
SEQ ID NO: 65
(RKRQDIDLGVSRKR)
SEQ ID NO: 66
(KRRQDIDLGVSRRK)
SEQ ID NO: 67
(RQDIDLGVSRKKRR)
SEQ ID NO: 68
(RQDIDLGVSRRRKK)
SEQ ID NO: 69
(KKRRRQDIDLGVSR)
SEQ ID NO: 70
(RRKKRQDIDLGVSR)
SEQ ID NO: 71
(RQDIDLGVSRGSGSGRR)
SEQ ID NO: 72
(GSGSGRRRQDIDLGVSR)
SEQ ID NO: 73
(RQDIDLGVSRGSGSGKK)
SEQ ID NO: 74
(GSGSGKKRQDIDLGVSR)
SEQ ID NO: 75
(YGRKKRRRQDIDLGVSR)
or
SEQ ID NO: 76
(RQDIDLGVSRYGRKKRR).

13. A polymer comprising:
a first polymer segment comprising at least 2 first repeating units; wherein each of the first repeating units of the first polymer segment comprises a first polymer backbone group directly or indirectly covalently linked to a first polymer side chain group comprising a peptide;
wherein the peptide comprises a sequence having 75% or greater sequence identity with a 6 to 10 amino acid fragment of SEQ ID NO: 218 (MDLIDILWRQDIDLGVSREVFDFS).

14. The polymer of claim 13, wherein the peptide comprises a sequence having 85% or greater sequence identity with a 6 to 10 amino acid fragment of SEQ ID NO: 218 (MDLIDILWRQDIDLGVSREVFDFS).

15. The polymer of claim 13, wherein the peptide comprises a 6 to 10 amino acid fragment of SEQ ID NO: 218 (MDLIDILWRQDIDLGVSREVFDFS).

16. The polymer of claim 13, wherein the peptide comprises a sequence having 75% or greater sequence identity of SEQ ID NO: 1 (RQDIDLGVSR), SEQ ID NO: 220 (ILWRQDIDLGVSR), or SEQ ID NO: 222 (LWRQDIDLGVSR).

17. The polymer of claim 16, wherein the peptide comprises a sequence having 85% or greater sequence identity of SEQ ID NO: 1 (RQDIDLGVSR), SEQ ID NO: 220 (ILWRQDIDLGVSR), or SEQ ID NO: 222 (LWRQDIDLGVSR).

18. The polymer of claim 16 or claim 17, wherein the peptide comprises SEQ ID NO: 1 (RQDIDLGVSR), SEQ ID NO: 220 (ILWRQDIDLGVSR), or SEQ ID NO: 222 (LWRQDIDLGVSR).

19. The polymer of any one of claims 13-18, wherein the peptide further comprises a charge modulating domain.

20. The polymer of claim 19, wherein the charge modulating domain has from 1 to 7 amino acid residues.

21. The polymer of claim 19 or claim 20, wherein the charge modulating domain is a glycine-serine domain, a cationic residue domain, or a combination thereof.

22. The polymer of claim 21, wherein the cationic residue domain is from 1 to 7 amino acid residues consisting of lysine, arginine, histidine, or a combination thereof.

23. The polymer of any one of claims 13-22, wherein the peptide has a net positive charge.

24. The polymer of any one of claims 13-23, the peptide having from 11 to 14 amino acid residues.

25. The polymer of any one of claims 16-24, wherein the peptide comprises a sequence having 85% or greater sequence identity of SEQ ID NO: 1 (RQDIDLGVSR).

26. The polymer of claim 25, wherein the sequence having 85% or greater sequence identity of SEQ ID NO: 1 (RQDIDLGVSR) is SEQ ID NO: 1 (RQDIDLGVSR).

27. The polymer of any one of claims 13-26, wherein the peptide is
SEQ ID NO: 2
(RQDIDLGVSRR)
SEQ ID NO: 3
(RRQDIDLGVSR)
SEQ ID NO: 4
(RQDIDLGVSRK)
SEQ ID NO: 5
(KRQDIDLGVSR)
SEQ ID NO: 6
(KRQDIDLGVSRR)
SEQ ID NO: 7
(RRQDIDLGVSRK)
SEQ ID NO: 8
(RQDIDLGVSRRR)
SEQ ID NO: 9
(RQDIDLGVSRRRR)
SEQ ID NO: 10
(RQDIDLGVSRRRRR)
SEQ ID NO: 11
(RQDIDLGVSRRRRRR)
SEQ ID NO: 12
(RRRQDIDLGVSR)
SEQ ID NO: 13
(RRRRQDIDLGVSR)
SEQ ID NO: 14
(RRRRRQDIDLGVSR)
SEQ ID NO: 15
(RRRRRRQDIDLGVSR)
SEQ ID NO: 16
(RRRQDIDLGVSRRR)
SEQ ID NO: 17
(RRRRQDIDLGVSRRRR)
SEQ ID NO: 18
(RRQDIDLGVSRR)
SEQ ID NO: 19
(RRQDIDLGVSRRR)
SEQ ID NO: 20
(RRRQDIDLGVSRR)
SEQ ID NO: 21
(RRQDIDLGVSRRRR)
SEQ ID NO: 22
(RRRRQDIDLGVSRR)
SEQ ID NO: 23
(RRQDIDLGVSRRRRR)
SEQ ID NO: 24
(RRRQDIDLGVSRRRR)
SEQ ID NO: 25
(RRRRQDIDLGVSRRR)
SEQ ID NO: 26
(RRRRRQDIDLGVSRR)
SEQ ID NO: 27
(RRQDIDLGVSRRRRRR)
SEQ ID NO: 28
(RRRQDIDLGVSRRRRR)
SEQ ID NO: 29
(RRRRRQDIDLGVSRRR)
SEQ ID NO: 30
(RRRRRRQDIDLGVSRR)
SEQ ID NO: 31
(RQDIDLGVSRKK)
SEQ ID NO: 32
(RQDIDLGVSRKKK)
SEQ ID NO: 33
(RQDIDLGVSRKKKK)
SEQ ID NO: 34
(RQDIDLGVSRKKKKK)
SEQ ID NO: 35
(KKRQDIDLGVSR)
SEQ ID NO: 36
(KKKRQDIDLGVSR)
SEQ ID NO: 37
(KKKKRQDIDLGVSR)
SEQ ID NO: 38
(KKKKKRQDIDLGVSR)
SEQ ID NO: 39
(KKRQDIDLGVSRKK)
SEQ ID NO: 40
(KKKRQDIDLGVSRKKK)
SEQ ID NO: 41
(KRQDIDLGVSRK)
SEQ ID NO: 42
(KRQDIDLGVSRKK)
SEQ ID NO: 43
(KKRQDIDLGVSRK)
SEQ ID NO: 44
(KRQDIDLGVSRKKK)
SEQ ID NO: 45
(KKKRQDIDLGVSRK)
SEQ ID NO: 46
(KRQDIDLGVSRKKKK)
SEQ ID NO: 47
(KKRQDIDLGVSRKKK)
SEQ ID NO: 48
(KKKRQDIDLGVSRKK)
SEQ ID NO: 49
(KKKKRQDIDLGVSRK)
SEQ ID NO: 50
(KRQDIDLGVSRKKKKK)
SEQ ID NO: 51
(KKRQDIDLGVSRKKKK)
SEQ ID NO: 52
(KKKKRQDIDLGVSRKK)
SEQ ID NO: 53
(KKKKKRQDIDLGVSRK)
SEQ ID NO: 54
(RQDIDLGVSRKRKR)
SEQ ID NO: 55
(KRKRRQDIDLGVSR)
SEQ ID NO: 56
(RKRKRQDIDLGVSR)
SEQ ID NO: 57
(RQDIDLGVSRRKRK)
SEQ ID NO: 58
(KKRQDIDLGVSRRR)
SEQ ID NO: 59
(RRRQDIDLGVSRKK)
SEQ ID NO: 60
(KRQDIDLGVSRRRR)
SEQ ID NO: 61
(KKKRQDIDLGVSRR)
SEQ ID NO: 62
(RRRRQDIDLGVSRK)
SEQ ID NO: 63
(KRRQDIDLGVSRKR)
SEQ ID NO: 64
(RKRQDIDLGVSRRK)
SEQ ID NO: 65
(RKRQDIDLGVSRKR)
SEQ ID NO: 66
(KRRQDIDLGVSRRK)
SEQ ID NO: 67
(RQDIDLGVSRKKRR)
SEQ ID NO: 68
(RQDIDLGVSRRRKK)
SEQ ID NO: 69
(KKRRRQDIDLGVSR)
SEQ ID NO: 70
(RRKKRQDIDLGVSR)
SEQ ID NO: 71
(RQDIDLGVSRGSGSGRR)
SEQ ID NO: 72
(GSGSGRRRQDIDLGVSR)
SEQ ID NO: 73
(RQDIDLGVSRGSGSGKK)
SEQ ID NO: 74
(GSGSGKKRQDIDLGVSR)
SEQ ID NO: 75
(YGRKKRRRQDIDLGVSR)
or
SEQ ID NO: 76
(RQDIDLGVSRYGRKKRR).

28. The polymer of any one of claims 13-27, wherein the polymer is a homopolymer.

29. The polymer of any one of claims 13-27, wherein the polymer is a copolymer.

30. The polymer of claim 29, wherein the copolymer is a block copolymer, random copolymer, or a statistical copolymer, preferably wherein the copolymer is a block copolymer.

31. The polymer of any one of claims 13-27, wherein the polymer is a brush polymer.

32. The polymer of claim 31, wherein the brush polymer is a high-density brush polymer characterized by a brush density greater than or equal to 50%.

33. The polymer of claim 31, wherein the brush polymer is a high-density brush polymer characterized by a brush density greater than or equal to 70%.

34. The polymer of claim 31, wherein the brush polymer is a high-density brush polymer characterized by a brush density greater than or equal to 90%.

35. The polymer of any one of claims 13-34, wherein the first polymer segment comprises at least 5 first repeating units.

36. The polymer of claim 35, wherein the first polymer segment comprises 5 to 30 first repeating units.

37. The polymer of any one of claims 13-36, wherein the polymer is characterized by a degree of polymerization of 2 to 1000.

38. The polymer of any one of claims 13-37, wherein the polymer is characterized by a polydispersity index less than 1.75.

39. The polymer of any one of claims 13-38, wherein at least a portion of the peptide is linked to the polymer backbone group via an enzymatically degradable linker.

40. The polymer of claim 39, wherein the enzymatically degradable linker is a matrix metalloproteinase (MMP) cleavage sequence, cathepsin B cleavage sequence, ester bond, reductive sensitive bond-disulfide bond, pH sensitive bond-imine bond or any combinations of these.

41. The polymer of any one of claims 13-40, wherein at least a portion of the peptide side-chain is linked to the polymer backbone or consists of a degradable or triggerable linker.

42. The polymer of any one of claims 13-41, wherein the polymer further comprises a tag for imaging and/or analysis, one or more additional peptides and/or proteins, a nonionic polymer, or a combination thereof.

43. The polymer of any one of claims 13-42 characterized by the formula (FX1a), (FX1b), (FX1c), (FX1d); (FX1e); (FX1f); or (FX1g):
Q¹-T-Q² (FX1a);
Q¹-T-[S]ₕ-Q² (FX1b);
Q¹-[S]ₕ-T-Q² (FX1c);
Q¹-[S]ᵢ-T-[S]ₕ-Q² (FX1d);
Q¹-[S]ᵢ-T-[S]ₕ-T-Q² (FX1e);
Q¹-T-[S]ᵢ-T-[S]ₕ-Q² (FX1f);
or
Q¹-T-[S]ᵢ-T-[S]ₕ-T-Q² (FX1g);
wherein each T is independently the first polymer segment comprising the first repeating units and each S is independently an additional polymer segment; Q¹ is a first backbone terminating group; Q² is a second backbone terminating group; and wherein h is zero or an integer selected over the range of 1 to 1000 and i is zero or an integer selected over the range of 1 to 1000.

44. The polymer of claim 43, wherein each -T- is independently —[Y¹]ₘ—; wherein each Y¹ is independently the first repeating unit of the first polymer segment; and each m is independently an integer selected from the range 0 to 1000, provided that at least one m is an integer selected from the range 1 to 1000.

45. The polymer of any one of claims 13-44 characterized by the formula (FX2a), (FX2b), or (FX2c): 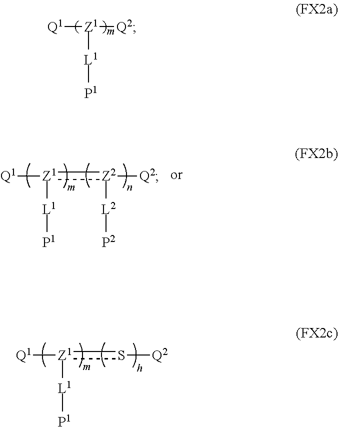
wherein
each Z¹ is independently a first polymer backbone group and each Z² is independently a second polymer backbone group;
each S is independently a repeating unit having a composition different from the first repeating unit;
Q′ is a first backbone terminating group and Q² is a second backbone terminating group;
each L¹ is independently a first linking group, each L² is independently a second linking group;
each P¹ is the peptide; wherein each P² is a polymer side chain having a composition different from that of P¹;
each m is independently an integer selected from the range of 2 to 1000;
each n is independently an integer selected from the range of 0 to 1000; and
each h is independently an integer selected from the range of 0 to 1000.

46. The polymer of any one of claims 13-45, wherein each of the first polymer backbone group and/or the second polymer backbone group is a substituted or unsubstituted polymerized norbornene, olefin, cyclic olefin, norbornene anhydride, cyclooctene, cyclopentadiene, styrene, acrylamide, or acrylate.

47. The polymer of any one of claims 13-45, wherein each of the first polymer backbone group and/or the second polymer backbone group is a polymerized norbornene dicarboxyimide monomer.

48. The polymer of any one of claims 13-45, wherein each polymer backbone group of the polymer is a polymerized norbornene dicarboxyimide monomer.

49. The polymer of any one of claims 13-45, wherein each of the first polymer backbone group and/or the second polymer backbone group is a polymerized acrylamide monomer, preferably a polymerized methacrylamide monomer.

50. The polymer of any one of claims 13-45, wherein each polymer backbone group of the polymer is a polymerized acrylamide monomer, preferably a polymerized methacrylamide monomer.

51. The polymer of claim 45, wherein each of Z¹ and Z² is independently a substituted or unsubstituted polymerized norbornene, oxanorbornene, olefin, cyclic olefin, cyclooctene, or cyclopentadiene.

52. The polymer of claim 45, wherein each Z¹ connected to L¹, and P¹ or a combination thereof is independently characterized by the formula (FX3a) or (FX3b): 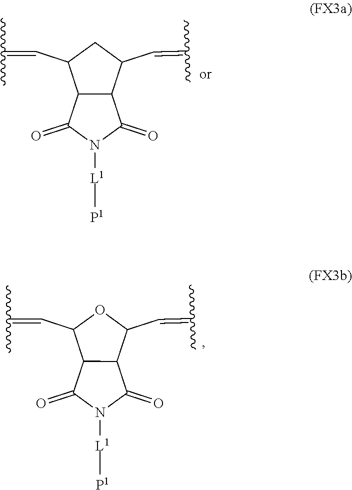
wherein each Z² connected to L², and P² or a combination thereof is independently characterized by the formula (FX4a) or (FX4b)

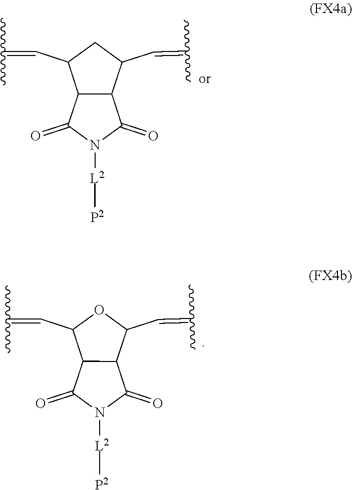

53. The polymer of claim 45, wherein each Z¹ connected to L¹, and P¹ or a combination thereof is independently characterized by the formula (FX3a):

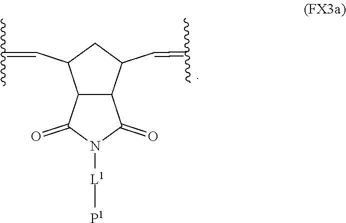

54. The polymer of claim 45 or claim 53, wherein each Z² connected to L², and P² or a combination thereof is independently characterized by the formula (FX4a):

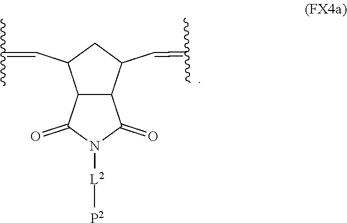

55. The polymer of any one of claims 45-54, wherein each of Q¹ and Q² is independently selected from a hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀alkylaryl, —CO₂R³, —CONR⁴R⁵, —COR⁶, —SOR⁷, —OSR⁷, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ is independently H, C₅-C₁₀ aryl or C₁-C₁₀ alkyl.

56. The polymer of any one of claims 45-55, wherein each of L¹ and L² is independently selected from a single bond, an oxygen, and groups having an alkyl group, an alkenylene group, an arylene group, an alkoxy group, an acyl group, a triazole group, a diazole group, a pyrazole group, and combinations thereof.

57. The polymer of claim 56, wherein each of L¹ and L² is independently selected from a single bond, —O—, C₁-C₁₀ alkyl, C₂-C₁₀ alkenylene, C₃-C₁₀ arylene, C₁-C₁₀ alkoxy, C₁-C₁₀ acyl and combinations thereof.

58. The polymer of any one of claims 13-57, wherein the polymer is stable against enzymatic digestion.

59. The polymer of any one of claims 13-58, wherein the polymer is stable against enzymatic digestion by a metalloproteinase.

60. The polymer of any one of claims 13-59, wherein the polymer is stable against enzymatic digestion by matrix metalloproteinases and thermolysin.

61. The polymer of any one of claims 13-60, wherein the polymer further comprises:
a second polymer segment comprising at least 2 second repeating units; wherein each of the second repeating units of the second polymer segment comprises a second polymer backbone group directly or indirectly covalently linked to a second polymer side chain group comprising a second peptide;
wherein the second peptide comprises a sequence having 75% or greater sequence identity of SEQ ID NO: 77 (LDEETGEFL).

62. The polymer of claim 61, wherein the second peptide comprises a sequence having 85% or greater sequence identity of SEQ ID NO: 77 (LDEETGEFL).

63. The polymer of claim 61 or claim 62, wherein the second peptide comprises SEQ ID NO: 77 (LDEETGEFL).

64. The polymer of claim 61, wherein the sequence having 75% or greater sequence identity of SEQ ID NO: 77 (LDEETGEFL) has a point mutation to comprise a proline residue.

65. The polymer of claim 61 or claim 62, wherein the second peptide comprises SEQ ID NO: 78 (LDPETGEFL).

66. The polymer of claim 61 or claim 64, wherein the sequence having 75% or greater sequence identity of SEQ ID NO: 77 (LDEETGEFL) has a point mutation to delete a glutamate residue.

67. The polymer of claim 66, wherein the second peptide comprises SEQ ID NO: 79 (LDPTGEFL) or SEQ ID NO: 80 (LDPETGFL).

68. The polymer of any one of claims 61-67, wherein the second peptide further comprises a second charge modulating domain.

69. The polymer of claim 68, wherein the second charge modulating domain has from 2 to 7 amino acid residues.

70. The polymer of claim 68 or claim 69, wherein the second charge modulating domain is a second glycine-serine domain, a second cationic residue domain, or a combination thereof.

71. The polymer of claim 70, wherein the second cationic residue domain is from 2 to 7 amino acid residues consisting of lysine, arginine, histidine, or a combination thereof.

72. The polymer of any one of claims 61-71, wherein the second peptide has a net positive charge.

73. The polymer of any one of claims 61-72, the second peptide having from 11 to 16 amino acid residues.

74. The polymer of any one of claims 61-73, wherein the second peptide is
SEQ ID NO: 81
(LDEETGEFLRR)
SEQ ID NO: 82
(LDEETGEFLRRR)
SEQ ID NO: 83
(LDEETGEFLRRRR)
SEQ ID NO: 84
(LDEETGEFLRRRRR)
SEQ ID NO: 85
(RRLDEETGEFL)
SEQ ID NO: 86
(RRRLDEETGEFL)
SEQ ID NO: 87
(RRRRLDEETGEFL)
SEQ ID NO: 88
(RRRRRLDEETGEFL)
SEQ ID NO: 89
(RRLDEETGEFLRR)
SEQ ID NO: 90
(RRRLDEETGEFLRRR)
SEQ ID NO: 91
(RLDEETGEFLR)
SEQ ID NO: 92
(RLDEETGEFLRR)
SEQ ID NO: 93
(RRLDEETGEFLR)
SEQ ID NO: 94
(RLDEETGEFLRRR)
SEQ ID NO: 95
(RRRLDEETGEFLR)
SEQ ID NO: 96
(RLDEETGEFLRRRR)
SEQ ID NO: 97
(RRLDEETGEFLRRR)
SEQ ID NO: 98
(RRRLDEETGEFLRR)
SEQ ID NO: 99
(RRRRLDEETGEFLR)
SEQ ID NO: 100
(RLDEETGEFLRRRRR)
SEQ ID NO: 101
(RRLDEETGEFLRRRR)
SEQ ID NO: 102
(RRRRLDEETGEFLRR)
SEQ ID NO: 103
(RRRRRLDEETGEFLR)
SEQ ID NO: 104
(LDEETGEFLKK)
SEQ ID NO: 105
(LDEETGEFLKKK)
SEQ ID NO: 106
(LDEETGEFLKKKK)
SEQ ID NO: 107
(LDEETGEFLKKKKK)
SEQ ID NO: 108
(KKLDEETGEFL)
SEQ ID NO: 109
(KKKLDEETGEFL)
SEQ ID NO: 110
(KKKKLDEETGEFL)
SEQ ID NO: 111
(KKKKKLDEETGEFL)
SEQ ID NO: 112
(KKLDEETGEFLKK)
SEQ ID NO: 113
(KKKLDEETGEFLKKK)
SEQ ID NO: 114
(KLDEETGEFLK)
SEQ ID NO: 115
(KLDEETGEFLKK)
SEQ ID NO: 116
(KKLDEETGEFLK)
SEQ ID NO: 117
(KLDEETGEFLKKK)
SEQ ID NO: 118
(KKKLDEETGEFLK)
SEQ ID NO: 119
(KLDEETGEFLKKKK)
SEQ ID NO: 120
(KKLDEETGEFLKKK)
SEQ ID NO: 121
(KKKLDEETGEFLKK)
SEQ ID NO: 122
(KKKKLDEETGEFLK)
SEQ ID NO: 123
(KLDEETGEFLKKKKK)
SEQ ID NO: 124
(KKLDEETGEFLKKKK)
SEQ ID NO: 125
(KKKKLDEETGEFLKK)
SEQ ID NO: 126
(KKKKKLDEETGEFLK)
SEQ ID NO: 127
(LDEETGEFLKRKR)
SEQ ID NO: 128
(KRKRLDEETGEFL)
SEQ ID NO: 129
(RKRKLDEETGEFL)
SEQ ID NO: 130
(LDEETGEFLRKRK)
SEQ ID NO: 131
(KKLDEETGEFLRR)
SEQ ID NO: 132
(RRLDEETGEFLKK)
SEQ ID NO: 133
(KLDEETGEFLRRR)
SEQ ID NO: 134
(KKKLDEETGEFLR)
SEQ ID NO: 135
(RRRLDEETGEFLK)
SEQ ID NO: 136
(KRLDEETGEFLKR)
SEQ ID NO: 137
(RKLDEETGEFLRK)
SEQ ID NO: 138
(RKLDEETGEFLKR)
SEQ ID NO: 139
(KRLDEETGEFLRK)
SEQ ID NO: 140
(LDEETGEFLKKRR)
SEQ ID NO: 141
(LDEETGEFLRRKK)
SEQ ID NO: 142
(KKRRLDEETGEFL)
SEQ ID NO: 143
(RRKKLDEETGEFL)
SEQ ID NO: 144
(LDEETGEFLGSGSGRR)
SEQ ID NO: 145
(GSGSGRRLDEETGEFL)
SEQ ID NO: 146
(LDEETGEFLGSGSGKK)
SEQ ID NO: 147
(GSGSGKKLDEETGEFL)
SEQ ID NO: 148
(LDPETGEFLRR)
SEQ ID NO: 149
(LDPETGEFLRRR)
SEQ ID NO: 150
(LDPETGEFLRRRR)
SEQ ID NO: 151
(LDPETGEFLRRRRR)
SEQ ID NO: 152
(RRLDPETGEFL)
SEQ ID NO: 153
(RRRLDPETGEFL)
SEQ ID NO: 154
(RRRRLDPETGEFL)
SEQ ID NO: 155
(RRRRRLDPETGEFL)
SEQ ID NO: 156
(RRLDPETGEFLRR)
SEQ ID NO: 157
(RRRLDPETGEFLRRR)
SEQ ID NO: 158
(RLDPETGEFLR)
SEQ ID NO: 159
(RLDPETGEFLRR)
SEQ ID NO: 160
(RRLDPETGEFLR)
SEQ ID NO: 161
(RLDPETGEFLRRR)
SEQ ID NO: 162
(RRRLDPETGEFLR)
SEQ ID NO: 163
(RLDPETGEFLRRRR)
SEQ ID NO: 164
(RRLDPETGEFLRRR)
SEQ ID NO: 165
(RRRLDPETGEFLRR)
SEQ ID NO: 166
(RRRRLDPETGEFLR)
SEQ ID NO: 167
(RLDPETGEFLRRRRR)
SEQ ID NO: 168
(RRLDPETGEFLRRRR)
SEQ ID NO: 169
(RRRRLDPETGEFLRR)
SEQ ID NO: 170
(RRRRRLDPETGEFLR)
SEQ ID NO: 171
(LDPETGEFLKK)
SEQ ID NO: 172
(LDPETGEFLKKK)
SEQ ID NO: 173
(LDPETGEFLKKKK)
SEQ ID NO: 174
(LDPETGEFLKKKKK)
SEQ ID NO: 175
(KKLDPETGEFL)
SEQ ID NO: 176
(KKKLDPETGEFL)
SEQ ID NO: 177
(KKKKLDPETGEFL)
SEQ ID NO: 178
(KKKKKLDPETGEFL)
SEQ ID NO: 179
(KKLDPETGEFLKK)
SEQ ID NO: 180
(KKKLDPETGEFLKKK)
SEQ ID NO: 181
(KLDPETGEFLK)
SEQ ID NO: 182
(KLDPETGEFLKK)
SEQ ID NO: 183
(KKLDPETGEFLK)
SEQ ID NO: 184
(KLDPETGEFLKKK)
SEQ ID NO: 185
(KKKLDPETGEFLK)
SEQ ID NO: 186
(KLDPETGEFLKKKK)
SEQ ID NO: 187
(KKLDPETGEFLKKK)
SEQ ID NO: 188
(KKKLDPETGEFLKK)
SEQ ID NO: 189
(KKKKLDPETGEFLK)
SEQ ID NO: 190
(KLDPETGEFLKKKKK)
SEQ ID NO: 191
(KKLDPETGEFLKKKK)
SEQ ID NO: 192
(KKKKLDPETGEFLKK)
SEQ ID NO: 193
(KKKKKLDPETGEFLK)
SEQ ID NO: 194
(LDPETGEFLKRKR)
SEQ ID NO: 195
(KRKRLDPETGEFL)
SEQ ID NO: 196
(RKRKLDPETGEFL)
SEQ ID NO: 197
(LDPETGEFLRKRK)
SEQ ID NO: 198
(KKLDPETGEFLRR)
SEQ ID NO: 199
(RRLDPETGEFLKK)
SEQ ID NO: 200
(KLDPETGEFLRRR)
SEQ ID NO: 201
(KKKLDPETGEFLR)
SEQ ID NO: 202
(RRRLDPETGEFLK)
SEQ ID NO: 203
(KRLDPETGEFLKR)
SEQ ID NO: 204
(RKLDPETGEFLRK)
SEQ ID NO: 205
(RKLDPETGEFLKR)
SEQ ID NO: 206
(KRLDPETGEFLRK)
SEQ ID NO: 207
(LDPETGEFLKKRR)
SEQ ID NO: 208
(LDPETGEFLRRKK)
SEQ ID NO: 209
(KKRRLDPETGEFL)
SEQ ID NO: 210
(RRKKLDPETGEFL)
SEQ ID NO: 211
(LDPETGEFLGSGSGRR)
SEQ ID NO: 212
(GSGSGRRLDPETGEFL)
SEQ ID NO: 213
(LDPETGEFLGSGSGKK)
SEQ ID NO: 214
(GSGSGKKLDPETGEFL)
SEQ ID NO: 215
(YGRKKRRLDPETGEFL)
or
SEQ ID NO: 216
(LDPETGEFLYGRKKRR).

75. A pharmaceutical composition comprising the peptide of any one of claims 1-12 or the polymer of any one of claims 13-74, and a pharmaceutically acceptable excipient.

76. A method of treating or managing a condition comprising administering to a subject an effective amount of the peptide of any one of claims 1-12, the polymer of any one of claims 13-74, or the pharmaceutical composition of claim 75.

77. The method of treating or managing a condition of claim 76, wherein the peptide of any one of claims 1-12, the polymer of any one of claims 13-74, or the pharmaceutical composition of claim 75 is administered intravenously, subcutaneously, intramuscularly, topically, orally, or a combination thereof.

78. The method of treating or managing a condition of claim 76 or claim 77, wherein the condition is associated with an inflammatory state, increased oxidative stress, autoimmune pathophysiology, chemo-preventative measures, neurodegeneration, or a combination thereof.

79. The method of treating or managing a condition of any one of claims 76-78, wherein the condition is an autoimmune disease.

80. The method of treating or managing a condition of claim 79, wherein the autoimmune disease is multiple sclerosis, systemic lupus erythematous, Sjogren syndrome, rheumatoid arthritis, vitiligo, or psoriasis.

81. The method of treating or managing a condition of any one of claims 76-78, wherein the condition is a respiratory disease.

82. The method of treating or managing a condition of claim 81, wherein the respiratory disease is COPD, emphysema, potential treatment for smokers, idiopathic pulmonary fibrosis, chronic sarcoidosis, or hypersensitivity pneumonitis.

83. The method of treating or managing a condition of any one of claims 76-78, wherein the condition is a gastrointestinal disease.

84. The method of treating or managing a condition of claim 83, wherein the gastrointestinal disease is ulcerative colitis, ulcers, prevent acetaminophen toxicity, non-alcoholic steatohepatitis, primary biliary cholangitis, cirrhosis, type 2 diabetes, or diabetic nephropathy.

85. The method of treating or managing a condition of any one of claims 76-78, wherein the condition is a cardiovascular disease.

86. The method of treating or managing a condition of claim 85, wherein the cardiovascular disease is cardiac ischemia-reperfusion injury, heart failure, or atherosclerosis.

87. The method of treating or managing a condition of any one of claims 76-78, wherein the condition is a neurodegenerative disease.

88. The method of treating or managing a condition of claim 87, wherein the neurodegenerative disease is Alzheimer's disease, Parkinson's disease, amyotrophic lateral sclerosis (ALS), Huntington's disease, Friedreich ataxia, or frontotemporal lobar degeneration.

89. The method of treating or managing a condition of any one of claims 76-78, wherein the method interrupts the protein-protein interaction between Nuclear factor (erythroid-derived 2)-like 2 (Nrf2) and Kelch-like ECH-Associating protein 1 (Keap1).

90. The method of any one of claims 76-89, further comprising contacting a target tissue of the subject with the polymer or a metabolite or product thereof.

91. The method of any one of claims 76-89, further comprising contacting a target cell of the subject with the polymer or a metabolite or product thereof.

92. The method of any one of claims 76-89, further comprising contacting a target receptor of the subject with the polymer or a metabolite or product thereof.

93. The method of any one of claims 76-89, further comprising contacting a target receptor through two or more target receptor domains of the subject with the polymer or a metabolite or product thereof.

94. The method of any one of claims 76-93, wherein the polymer passes through the cell membrane and contacts an intracellular target.


SEQUENCE LISTING (225 sequences)

SEQ ID NO: 1 - protein, 10 aa, synthetic construct
RQDIDLGVSR

SEQ ID NO: 2 - protein, 11 aa, synthetic construct
RQDIDLGVSRR

SEQ ID NO: 3 - protein, 11 aa, synthetic construct
RRQDIDLGVSR

SEQ ID NO: 4 - protein, 11 aa, synthetic construct
RQDIDLGVSRK

SEQ ID NO: 5 - protein, 11 aa, synthetic construct
KRQDIDLGVSR

SEQ ID NO: 6 - protein, 12 aa, synthetic construct
KRQDIDLGVSRR

SEQ ID NO: 7 - protein, 12 aa, synthetic construct
RRQDIDLGVSRK

SEQ ID NO: 8 - protein, 12 aa, synthetic construct
RQDIDLGVSRRR

SEQ ID NO: 9 - protein, 13 aa, synthetic construct
RQDIDLGVSRRRR

SEQ ID NO: 10 - protein, 14 aa, synthetic construct
RQDIDLGVSRRRRR

SEQ ID NO: 11 - protein, 15 aa, synthetic construct
RQDIDLGVSRRRRRR

SEQ ID NO: 12 - protein, 12 aa, synthetic construct
RRRQDIDLGVSR

SEQ ID NO: 13 - protein, 13 aa, synthetic construct
RRRRQDIDLGVSR

SEQ ID NO: 14 - protein, 14 aa, synthetic construct
RRRRRQDIDLGVSR

SEQ ID NO: 15 - protein, 15 aa, synthetic construct
RRRRRRQDIDLGVSR

SEQ ID NO: 16 - protein, 14 aa, synthetic construct
RRRQDIDLGVSRRR

SEQ ID NO: 17 - protein, 16 aa, synthetic construct
RRRRQDIDLGVSRRRR

SEQ ID NO: 18 - protein, 12 aa, synthetic construct
RRQDIDLGVSRR

SEQ ID NO: 19 - protein, 13 aa, synthetic construct
RRQDIDLGVSRRR

SEQ ID NO: 20 - protein, 13 aa, synthetic construct
RRRQDIDLGVSRR

SEQ ID NO: 21 - protein, 14 aa, synthetic construct
RRQDIDLGVSRRRR

SEQ ID NO: 22 - protein, 14 aa, synthetic construct
RRRRQDIDLGVSRR

SEQ ID NO: 23 - protein, 15 aa, synthetic construct
RRQDIDLGVSRRRRR

SEQ ID NO: 24 - protein, 15 aa, synthetic construct
RRRQDIDLGVSRRRR

SEQ ID NO: 25 - protein, 15 aa, synthetic construct
RRRRQDIDLGVSRRR

SEQ ID NO: 26 - protein, 15 aa, synthetic construct
RRRRRQDIDLGVSRR

SEQ ID NO: 27 - protein, 16 aa, synthetic construct
RRQDIDLGVSRRRRRR

SEQ ID NO: 28 - protein, 16 aa, synthetic construct
RRRQDIDLGVSRRRRR

SEQ ID NO: 29 - protein, 16 aa, synthetic construct
RRRRRQDIDLGVSRRR

SEQ ID NO: 30 - protein, 16 aa, synthetic construct
RRRRRRQDIDLGVSRR

SEQ ID NO: 31 - protein, 12 aa, synthetic construct
RQDIDLGVSRKK

SEQ ID NO: 32 - protein, 13 aa, synthetic construct
RQDIDLGVSRKKK

SEQ ID NO: 33 - protein, 14 aa, synthetic construct
RQDIDLGVSRKKKK

SEQ ID NO: 34 - protein, 15 aa, synthetic construct
RQDIDLGVSRKKKKK

SEQ ID NO: 35 - protein, 12 aa, synthetic construct
KKRQDIDLGVSR

SEQ ID NO: 36 - protein, 13 aa, synthetic construct
KKKRQDIDLGVSR

SEQ ID NO: 37 - protein, 14 aa, synthetic construct
KKKKRQDIDLGVSR

SEQ ID NO: 38 - protein, 15 aa, synthetic construct
KKKKKRQDIDLGVSR

SEQ ID NO: 39 - protein, 14 aa, synthetic construct
KKRQDIDLGVSRKK

SEQ ID NO: 40 - protein, 16 aa, synthetic construct
KKKRQDIDLGVSRKKK

SEQ ID NO: 41 - protein, 12 aa, synthetic construct
KRQDIDLGVSRK

SEQ ID NO: 42 - protein, 13 aa, synthetic construct
KRQDIDLGVSRKK

SEQ ID NO: 43 - protein, 13 aa, synthetic construct
KKRQDIDLGVSRK

SEQ ID NO: 44 - protein, 14 aa, synthetic construct
KRQDIDLGVSRKKK

SEQ ID NO: 45 - protein, 14 aa, synthetic construct
KKKRQDIDLGVSRK

SEQ ID NO: 46 - protein, 15 aa, synthetic construct
KRQDIDLGVSRKKKK

SEQ ID NO: 47 - protein, 15 aa, synthetic construct
KKRQDIDLGVSRKKK

SEQ ID NO: 48 - protein, 15 aa, synthetic construct
KKKRQDIDLGVSRKK

SEQ ID NO: 49 - protein, 15 aa, synthetic construct
KKKKRQDIDLGVSRK

SEQ ID NO: 50 - protein, 16 aa, synthetic construct
KRQDIDLGVSRKKKKK

SEQ ID NO: 51 - protein, 16 aa, synthetic construct
KKRQDIDLGVSRKKKK

SEQ ID NO: 52 - protein, 16 aa, synthetic construct
KKKKRQDIDLGVSRKK

SEQ ID NO: 53 - protein, 16 aa, synthetic construct
KKKKKRQDIDLGVSRK

SEQ ID NO: 54 - protein, 14 aa, synthetic construct
RQDIDLGVSRKRKR

SEQ ID NO: 55 - protein, 14 aa, synthetic construct
KRKRRQDIDLGVSR

SEQ ID NO: 56 - protein, 14 aa, synthetic construct
RKRKRQDIDLGVSR

SEQ ID NO: 57 - protein, 14 aa, synthetic construct
RQDIDLGVSRRKRK

SEQ ID NO: 58 - protein, 14 aa, synthetic construct
KKRQDIDLGVSRRR

SEQ ID NO: 59 - protein, 14 aa, synthetic construct
RRRQDIDLGVSRKK

SEQ ID NO: 60 - protein, 14 aa, synthetic construct
KRQDIDLGVSRRRR

SEQ ID NO: 61 - protein, 14 aa, synthetic construct
KKKRQDIDLGVSRR

SEQ ID NO: 62 - protein, 14 aa, synthetic construct
RRRRQDIDLGVSRK

SEQ ID NO: 63 - protein, 14 aa, synthetic construct
KRRQDIDLGVSRKR

SEQ ID NO: 64 - protein, 14 aa, synthetic construct
RKRQDIDLGVSRRK

SEQ ID NO: 65 - protein, 14 aa, synthetic construct
RKRQDIDLGVSRKR

SEQ ID NO: 66 - protein, 14 aa, synthetic construct
KRRQDIDLGVSRRK

SEQ ID NO: 67 - protein, 14 aa, synthetic construct
RQDIDLGVSRKKRR

SEQ ID NO: 68 - protein, 14 aa, synthetic construct
RQDIDLGVSRRRKK

SEQ ID NO: 69 - protein, 14 aa, synthetic construct
KKRRRQDIDLGVSR

SEQ ID NO: 70 - protein, 14 aa, synthetic construct
RRKKRQDIDLGVSR

SEQ ID NO: 71 - protein, 17 aa, synthetic construct
RQDIDLGVSRGSGSGRR

SEQ ID NO: 72 - protein, 17 aa, synthetic construct
GSGSGRRRQDIDLGVSR

SEQ ID NO: 73 - protein, 17 aa, synthetic construct
RQDIDLGVSRGSGSGKK

SEQ ID NO: 74 - protein, 17 aa, synthetic construct
GSGSGKKRQDIDLGVSR

SEQ ID NO: 75 - protein, 17 aa, synthetic construct
YGRKKRRRQDIDLGVSR

SEQ ID NO: 76 - protein, 17 aa, synthetic construct
RQDIDLGVSRYGRKKRR

SEQ ID NO: 77 - protein, 9 aa, synthetic construct
LDEETGEFL

SEQ ID NO: 78 - protein, 9 aa, synthetic construct
LDPETGEFL

SEQ ID NO: 79 - protein, 8 aa, synthetic construct
LDPTGEFL

SEQ ID NO: 80 - protein, 8 aa, synthetic construct
LDPETGFL

SEQ ID NO: 81 - protein, 11 aa, synthetic construct
LDEETGEFLRR

SEQ ID NO: 82 - protein, 12 aa, synthetic construct
LDEETGEFLRRR

SEQ ID NO: 83 - protein, 13 aa, synthetic construct
LDEETGEFLRRRR

SEQ ID NO: 84 - protein, 14 aa, synthetic construct
LDEETGEFLRRRRR

SEQ ID NO: 85 - protein, 11 aa, synthetic construct
RRLDEETGEFL

SEQ ID NO: 86 - protein, 12 aa, synthetic construct
RRRLDEETGEFL

SEQ ID NO: 87 - protein, 13 aa, synthetic construct
RRRRLDEETGEFL

SEQ ID NO: 88 - protein, 14 aa, synthetic construct
RRRRRLDEETGEFL

SEQ ID NO: 89 - protein, 13 aa, synthetic construct
RRLDEETGEFLRR

SEQ ID NO: 90 - protein, 15 aa, synthetic construct
RRRLDEETGEFLRRR

SEQ ID NO: 91 - protein, 11 aa, synthetic construct
RLDEETGEFLR

SEQ ID NO: 92 - protein, 12 aa, synthetic construct
RLDEETGEFLRR

SEQ ID NO: 93 - protein, 12 aa, synthetic construct
RRLDEETGEFLR

SEQ ID NO: 94 - protein, 13 aa, synthetic construct
RLDEETGEFLRRR

SEQ ID NO: 95 - protein, 13 aa, synthetic construct
RRRLDEETGEFLR

SEQ ID NO: 96 - protein, 14 aa, synthetic construct
RLDEETGEFLRRRR

SEQ ID NO: 97 - protein, 14 aa, synthetic construct
RRLDEETGEFLRRR

SEQ ID NO: 98 - protein, 14 aa, synthetic construct
RRRLDEETGEFLRR

SEQ ID NO: 99 - protein, 14 aa, synthetic construct
RRRRLDEETGEFLR

SEQ ID NO: 100 - protein, 15 aa, synthetic construct
RLDEETGEFLRRRRR

SEQ ID NO: 101 - protein, 15 aa, synthetic construct
RRLDEETGEFLRRRR

SEQ ID NO: 102 - protein, 15 aa, synthetic construct
RRRRLDEETGEFLRR

SEQ ID NO: 103 - protein, 15 aa, synthetic construct
RRRRRLDEETGEFLR

SEQ ID NO: 104 - protein, 11 aa, synthetic construct
LDEETGEFLKK

SEQ ID NO: 105 - protein, 12 aa, synthetic construct
LDEETGEFLKKK

SEQ ID NO: 106 - protein, 13 aa, synthetic construct
LDEETGEFLKKKK

SEQ ID NO: 107 - protein, 14 aa, synthetic construct
LDEETGEFLKKKKK

SEQ ID NO: 108 - protein, 11 aa, synthetic construct
KKLDEETGEFL

SEQ ID NO: 109 - protein, 12 aa, synthetic construct
KKKLDEETGEFL

SEQ ID NO: 110 - protein, 13 aa, synthetic construct
KKKKLDEETGEFL

SEQ ID NO: 111 - protein, 14 aa, synthetic construct
KKKKKLDEETGEFL

SEQ ID NO: 112 - protein, 13 aa, synthetic construct
KKLDEETGEFLKK

SEQ ID NO: 113 - protein, 15 aa, synthetic construct
KKKLDEETGEFLKKK

SEQ ID NO: 114 - protein, 11 aa, synthetic construct
KLDEETGEFLK

SEQ ID NO: 115 - protein, 12 aa, synthetic construct
KLDEETGEFLKK

SEQ ID NO: 116 - protein, 12 aa, synthetic construct
KKLDEETGEFLK

SEQ ID NO: 117 - protein, 13 aa, synthetic construct
KLDEETGEFLKKK

SEQ ID NO: 118 - protein, 13 aa, synthetic construct
KKKLDEETGEFLK

SEQ ID NO: 119 - protein, 14 aa, synthetic construct
KLDEETGEFLKKKK

SEQ ID NO: 120 - protein, 14 aa, synthetic construct
KKLDEETGEFLKKK

SEQ ID NO: 121 - protein, 14 aa, synthetic construct
KKKLDEETGEFLKK

SEQ ID NO: 122 - protein, 14 aa, synthetic construct
KKKKLDEETGEFLK

SEQ ID NO: 123 - protein, 15 aa, synthetic construct
KLDEETGEFLKKKKK

SEQ ID NO: 124 - protein, 15 aa, synthetic construct
KKLDEETGEFLKKKK

SEQ ID NO: 125 - protein, 15 aa, synthetic construct
KKKKLDEETGEFLKK

SEQ ID NO: 126 - protein, 15 aa, synthetic construct
KKKKKLDEETGEFLK

SEQ ID NO: 127 - protein, 13 aa, synthetic construct
LDEETGEFLKRKR

SEQ ID NO: 128 - protein, 13 aa, synthetic construct
KRKRLDEETGEFL

SEQ ID NO: 129 - protein, 13 aa, synthetic construct
RKRKLDEETGEFL

SEQ ID NO: 130 - protein, 13 aa, synthetic construct
LDEETGEFLRKRK

SEQ ID NO: 131 - protein, 13 aa, synthetic construct
KKLDEETGEFLRR

SEQ ID NO: 132 - protein, 13 aa, synthetic construct
RRLDEETGEFLKK

SEQ ID NO: 133 - protein, 13 aa, synthetic construct
KLDEETGEFLRRR

SEQ ID NO: 134 - protein, 13 aa, synthetic construct
KKKLDEETGEFLR

SEQ ID NO: 135 - protein, 13 aa, synthetic construct
RRRLDEETGEFLK

SEQ ID NO: 136 - protein, 13 aa, synthetic construct
KRLDEETGEFLKR

SEQ ID NO: 137 - protein, 13 aa, synthetic construct
RKLDEETGEFLRK

SEQ ID NO: 138 - protein, 13 aa, synthetic construct
RKLDEETGEFLKR

SEQ ID NO: 139 - protein, 13 aa, synthetic construct
KRLDEETGEFLRK

SEQ ID NO: 140 - protein, 13 aa, synthetic construct
LDEETGEFLKKRR

SEQ ID NO: 141 - protein, 13 aa, synthetic construct
LDEETGEFLRRKK

SEQ ID NO: 142 - protein, 13 aa, synthetic construct
KKRRLDEETGEFL

SEQ ID NO: 143 - protein, 13 aa, synthetic construct
RRKKLDEETGEFL

SEQ ID NO: 144 - protein, 16 aa, synthetic construct
LDEETGEFLGSGSGRR

SEQ ID NO: 145 - protein, 16 aa, synthetic construct
GSGSGRRLDEETGEFL

SEQ ID NO: 146 - protein, 16 aa, synthetic construct
LDEETGEFLGSGSGKK

SEQ ID NO: 147 - protein, 16 aa, synthetic construct
GSGSGKKLDEETGEFL

SEQ ID NO: 148 - protein, 11 aa, synthetic construct
LDPETGEFLRR

SEQ ID NO: 149 - protein, 12 aa, synthetic construct
LDPETGEFLRRR

SEQ ID NO: 150 - protein, 13 aa, synthetic construct
LDPETGEFLRRRR

SEQ ID NO: 151 - protein, 14 aa, synthetic construct
LDPETGEFLRRRRR

SEQ ID NO: 152 - protein, 11 aa, synthetic construct
RRLDPETGEFL

SEQ ID NO: 153 - protein, 12 aa, synthetic construct
RRRLDPETGEFL

SEQ ID NO: 154 - protein, 13 aa, synthetic construct
RRRRLDPETGEFL

SEQ ID NO: 155 - protein, 14 aa, synthetic construct
RRRRRLDPETGEFL

SEQ ID NO: 156 - protein, 13 aa, synthetic construct
RRLDPETGEFLRR

SEQ ID NO: 157 - protein, 15 aa, synthetic construct
RRRLDPETGEFLRRR

SEQ ID NO: 158 - protein, 11 aa, synthetic construct
RLDPETGEFLR

SEQ ID NO: 159 - protein, 12 aa, synthetic construct
RLDPETGEFLRR

SEQ ID NO: 160 - protein, 12 aa, synthetic construct
RRLDPETGEFLR

SEQ ID NO: 161 - protein, 13 aa, synthetic construct
RLDPETGEFLRRR

SEQ ID NO: 162 - protein, 13 aa, synthetic construct
RRRLDPETGEFLR

SEQ ID NO: 163 - protein, 14 aa, synthetic construct
RLDPETGEFLRRRR

SEQ ID NO: 164 - protein, 14 aa, synthetic construct
RRLDPETGEFLRRR

SEQ ID NO: 165 - protein, 14 aa, synthetic construct
RRRLDPETGEFLRR

SEQ ID NO: 166 - protein, 14 aa, synthetic construct
RRRRLDPETGEFLR

SEQ ID NO: 167 - protein, 15 aa, synthetic construct
RLDPETGEFLRRRRR

SEQ ID NO: 168 - protein, 15 aa, synthetic construct
RRLDPETGEFLRRRR

SEQ ID NO: 169 - protein, 15 aa, synthetic construct
RRRRLDPETGEFLRR

SEQ ID NO: 170 - protein, 15 aa, synthetic construct
RRRRRLDPETGEFLR

SEQ ID NO: 171 - protein, 11 aa, synthetic construct
LDPETGEFLKK

SEQ ID NO: 172 - protein, 12 aa, synthetic construct
LDPETGEFLKKK

SEQ ID NO: 173 - protein, 13 aa, synthetic construct
LDPETGEFLKKKK

SEQ ID NO: 174 - protein, 14 aa, synthetic construct
LDPETGEFLKKKKK

SEQ ID NO: 175 - protein, 11 aa, synthetic construct
KKLDPETGEFL

SEQ ID NO: 176 - protein, 12 aa, synthetic construct
KKKLDPETGEFL

SEQ ID NO: 177 - protein, 13 aa, synthetic construct
KKKKLDPETGEFL

SEQ ID NO: 178 - protein, 14 aa, synthetic construct
KKKKKLDPETGEFL

SEQ ID NO: 179 - protein, 13 aa, synthetic construct
KKLDPETGEFLKK

SEQ ID NO: 180 - protein, 15 aa, synthetic construct
KKKLDPETGEFLKKK

SEQ ID NO: 181 - protein, 11 aa, synthetic construct
KLDPETGEFLK

SEQ ID NO: 182 - protein, 12 aa, synthetic construct
KLDPETGEFLKK

SEQ ID NO: 183 - protein, 12 aa, synthetic construct
KKLDPETGEFLK

SEQ ID NO: 184 - protein, 13 aa, synthetic construct
KLDPETGEFLKKK

SEQ ID NO: 185 - protein, 13 aa, synthetic construct
KKKLDPETGEFLK

SEQ ID NO: 186 - protein, 14 aa, synthetic construct
KLDPETGEFLKKKK

SEQ ID NO: 187 - protein, 14 aa, synthetic construct
KKLDPETGEFLKKK

SEQ ID NO: 188 - protein, 14 aa, synthetic construct
KKKLDPETGEFLKK

SEQ ID NO: 189 - protein, 14 aa, synthetic construct
KKKKLDPETGEFLK

SEQ ID NO: 190 - protein, 15 aa, synthetic construct
KLDPETGEFLKKKKK

SEQ ID NO: 191 - protein, 15 aa, synthetic construct
KKLDPETGEFLKKKK

SEQ ID NO: 192 - protein, 15 aa, synthetic construct
KKKKLDPETGEFLKK

SEQ ID NO: 193 - protein, 15 aa, synthetic construct
KKKKKLDPETGEFLK

SEQ ID NO: 194 - protein, 13 aa, synthetic construct
LDPETGEFLKRKR

SEQ ID NO: 195 - protein, 13 aa, synthetic construct
KRKRLDPETGEFL

SEQ ID NO: 196 - protein, 13 aa, synthetic construct
RKRKLDPETGEFL

SEQ ID NO: 197 - protein, 13 aa, synthetic construct
LDPETGEFLRKRK

SEQ ID NO: 198 - protein, 13 aa, synthetic construct
KKLDPETGEFLRR

SEQ ID NO: 199 - protein, 13 aa, synthetic construct
RRLDPETGEFLKK

SEQ ID NO: 200 - protein, 13 aa, synthetic construct
KLDPETGEFLRRR

SEQ ID NO: 201 - protein, 13 aa, synthetic construct
KKKLDPETGEFLR

SEQ ID NO: 202 - protein, 13 aa, synthetic construct
RRRLDPETGEFLK

SEQ ID NO: 203 - protein, 13 aa, synthetic construct
KRLDPETGEFLKR

SEQ ID NO: 204 - protein, 13 aa, synthetic construct
RKLDPETGEFLRK

SEQ ID NO: 205 - protein, 13 aa, synthetic construct
RKLDPETGEFLKR

SEQ ID NO: 206 - protein, 13 aa, synthetic construct
KRLDPETGEFLRK

SEQ ID NO: 207 - protein, 13 aa, synthetic construct
LDPETGEFLKKRR

SEQ ID NO: 208 - protein, 13 aa, synthetic construct
LDPETGEFLRRKK

SEQ ID NO: 209 - protein, 13 aa, synthetic construct
KKRRLDPETGEFL

SEQ ID NO: 210 - protein, 13 aa, synthetic construct
RRKKLDPETGEFL

SEQ ID NO: 211 - protein, 16 aa, synthetic construct
LDPETGEFLGSGSGRR

SEQ ID NO: 212 - protein, 16 aa, synthetic construct
GSGSGRRLDPETGEFL

SEQ ID NO: 213 - protein, 16 aa, synthetic construct
LDPETGEFLGSGSGKK

SEQ ID NO: 214 - protein, 16 aa, synthetic construct
GSGSGKKLDPETGEFL

SEQ ID NO: 215 - protein, 16 aa, synthetic construct
YGRKKRRLDPETGEFL

SEQ ID NO: 216 - protein, 16 aa, synthetic construct
LDPETGEFLYGRKKRR

SEQ ID NO: 217 - protein, 285 aa, synthetic construct
RLIYTAGGYFRQSLSYLEAYNPSDGTWLRRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVATATWTFVAPMKHRRSALGITVHQGRIYVLGGYDGHTFLDSVECYDPDTDTWSEVTRMTSGRSGVGVAVT

SEQ ID NO: 218 - protein, 24 aa, synthetic construct
MDLIDILWRQDIDLGVSREVFDFS

SEQ ID NO: 219 - protein, 14 aa, synthetic construct
ILWRQDIDLGVSRR

SEQ ID NO: 220 - protein, 13 aa, synthetic construct
ILWRQDIDLGVSR

SEQ ID NO: 221 - protein, 13 aa, synthetic construct
LWRQDIDLGVSRR

SEQ ID NO: 222 - protein, 12 aa, synthetic construct
LWRQDIDLGVSR

SEQ ID NO: 223 - protein, 14 aa, synthetic construct
LWRQDIDLGVSREV

SEQ ID NO: 224 - protein, 16 aa, synthetic construct
MDLIDILWRQDIDLGV

SEQ ID NO: 225 - protein, 9 aa, synthetic construct
LWRQDIDLG